(19) United States
(12) Patent Application Publication
(10) Pub. No.: US 2022/0064167 A1
(43) Pub. Date: Mar. 3, 2022

(54) IMIDAZO-PYRAZOLE CARBOXAMIDE DERIVATIVES AS ANTICANCER AGENTS AND THE SYNTHESIS THEREOF

(71) Applicant: AVIDIN KFT., Szeged (HU)

(72) Inventors: András DEMJÉN, Szeged (HU); László PUSKÁS, Szeged (HU); Ivan KANIZSAI, Szeged (HU); Gábor SZEBENI, Nagyszénás (HU); Anikó ANGYAL, Szeged (HU); Márió GYURIS, Szeged (HU); László HACKLER, Szeged (HU)

(73) Assignee: AVIDIN KFT., Szeged (HU)

(21) Appl. No.: 17/056,267

(22) PCT Filed: May 17, 2019
(86) PCT No.: PCT/HU2019/000014

§ 371 Date: Nov. 17, 2020

(30) Foreign Application Priority Data
May 17, 2018 (HU) P1800170

(51) Int. Cl. C07D 487/04; A61P 35/00

(52) U.S. Cl. CPC C07D 487/04; A61P 35/00

(57) ABSTRACT

The present invention relates to novel imidazo[1,2-b]pyrazole carboxamide and carbothioamide derivatives of general formula (V), and the advantageous derivatives and pharmaceutically acceptable salts thereof, the synthesis thereof, and medicinal and/or pharmaceutical composition comprising these compounds thereof and synthesis thereof, and for use as a medicament, for use in the treatment of different diseases, advantageously of cancer. The subject compounds are advantageously for use in the treatment of solid malignancies, advantageously breast, lung, melanoma, gliomas, and myeloproliferative and myelodysplastic neoplasms, acute myelogenous/myeloid leukemias and colon cancer by the differentiation and subsequent apoptosis of pre-matured myeloid leukemic cells or myeloid-derived suppressor cells and/or by direct effect on solid tumors.

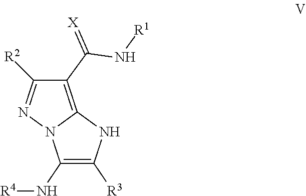

The present invention relates to novel imidazo[1,2-b]pyrazole carboxamide and carbothioamide derivatives and pharmaceutically acceptable salts thereof, the synthesis thereof, and medicinal and/or pharmaceutical composition comprising these compounds thereof and synthesis thereof, and for use as a medicament, for use in the treatment of different diseases, advantageously of cancer.

The subject compounds are advantageously for use in the treatment of solid malignancies, advantageously breast, lung, melanoma, gliomas, and myeloproliferative and myelodysplastic neoplasms, colon cancer, acute myelogenous/myeloid leukemias by the differentiation and subsequent apoptosis of pre-matured myeloid leukemic cells or myeloid-derived suppressor cells and/or by direct effect on solid tumors.

Our invention relates to novel bicyclic imidazo[1,2-b]pyrazole carboxamide and carbothioamide derivatives

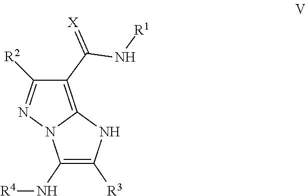

wherein in general formula (V)
R₁ represents hydrogen; branched or unbranched C1-C8-alkyl, aralkyl or aryl group advantageously optionally substituted phenyl or benzyl group; especially advantageously optionally substituted with 1; 2; 3; or 4 electron-withdrawing or electron-donating groups in ortho- metha and/or para positions;
furthermore represents heteroaryl groups and heterocycles in saturated or unsaturated forms containing O, N and/or S atoms; advantageously three-, four-, five-, six- and seven membered heterocyclic ring(s);
R₂ represents hydrogen and branched or un-branched C1-C8-alkyl group;
R₃ represents aliphatic branched or unbranched C1-C8-alkyl, advantageously tert-butyl, cyclopentyl, cyclohexyl group; aralkyl or aryl group advantageously optionally substituted phenyl or benzyl group; especially advantageously optionally substituted with 1; 2; 3; or 4 electron-withdrawing or electron-donating groups in ortho- metha and/or para positions;
furthermore represents heteroaryl groups and heterocycles in saturated or unsaturated forms containing O, N and/or S atoms, advantageously three-, four-, five-, six- and seven membered heterocyclic ring(s);
R₄ represents aliphatic branched or unbranched C1-C8-alkyl, advantageously methyl, n-pentyl, 1,1,3,3-tetramethylbutyl, tert-butyl group; CH2R′ group wherein R′ represents hydrogen, branched or unbranched C1-C8 alkyl group; CO(OR″) group, wherein R″ represents branched or unbranched C1-C8 alkyl, aralkyl or aryl group advantageously optionally substituted phenyl or benzyl group, especially advantageously optionally substituted with 1; 2; 3; or 4 electron-withdrawing or electron-donating groups in ortho- metha and/or para positions;
furthermore represents heteroaryl groups and heterocycles in saturated or unsaturated forms containing O, N and/or S atoms, advantageously three-, four-, five-, six- and seven membered heterocyclic ring(s); C(O)R′ group, wherein R′ represents heteroaryl group;
X represents O- or S-atom,
advantageously X represents O atom where the general formula is (IV) and

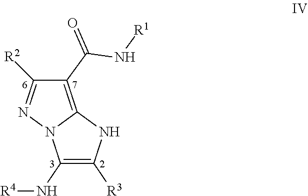

advantagously X represents S atom where the general formula is (IV′);

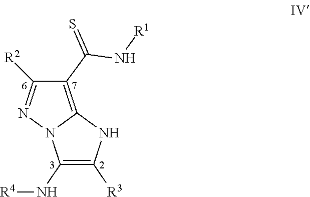

wherein R1 furthermore represents especially advantageously a 4-fluoro-, 4-N-dimethylamino-, 2,4-difluoro-, 4-aminophenyl, 4-SMe, 4-OH substituted phenyl group; unsubstituted phenyl group; furthermore represents advantageously O, N or N-heterocycles, especially advantageously isoxazole and 3-pyridyl group;
wherein R2 represents advantageously hydrogen;
wherein R3 represents an aliphatic C1-C8-alkyl group, advantageously branched alkyl chain, especially advantageously tert-butyl, 1,1,3,3-tetramethylbutyl and/or alicyclic cyclohexyl group;
wherein R4 represents an aliphatic C1-C8-alkyl group, advantageously branched alkyl chain especially advantageously tert-butyl, 1,1,3,3-tetramethylbutyl and cyclohexyl group.

The subject matter of the invention furthermore relates advantageously to novel bicyclic imidazo[1,2-b]pyrazole carboxamide derivatives of general formula (V) advantageously of general formula (IV) or (IV′) as listed detailed as follows

### Primary Carboxamide Derivatives and Pharmaceutically Acceptable Salts Thereof

3-(Tert-butylamino)-2-phenyl-1H-imidazo[1,2-b]pyrazole-7-carboxamide
2-Phenyl-3-((2,4,4-trimethylpentan-2-yl)amino)-1H-imidazo[1,2-b]pyrazole-7-carbox-amide
Methyl 2-((7-carbamoyl-2-phenyl-1H-imidazo[1,2-b]pyrazol-3-yl)amino)acetate
3-(Cyclohexylamino)-2-phenyl-1H-imidazo[1,2-b]pyrazole-7-carboxamide
3-((4-Methoxyphenyl)amino)-2-phenyl-1H-imidazo[1,2-b]pyrazole-7-carboxamide
2-(p-Tolyl)-3-((2,4,4-trimethylpentan-2-yl)amino)-1H-imidazo[1,2-b]pyrazole-7-carbox-amide
2-(4-Methoxyphenyl)-3-((2,4,4-trimethylpentan-2-yl)amino)-1H-imidazo[1,2-b]pyrazole-7-carbox-amide
4-(7-Carbamoyl-3-((2,4,4-trimethylpentan-2-yl)amino)-1H-imidazo[1,2-b]pyrazol-2-yl)-2-methoxy-phenyl acetate
Methyl 2-((7-carbamoyl-2-(2,4,6-trimethoxyphenyl)-1H-imidazo[1,2-b]pyrazol-3-yl) amino)acetate
2-(4-Fluorophenyl)-3-((2,4,4-trimethylpentan-2-yl)amino)-1H-imidazo[1,2-b]pyrazole-7-carboxamide
Methyl 2-((7-carbamoyl-2-(4-fluorophenyl)-1H-imidazo[1,2-b]pyrazol-3-yl)amino)acetate
2-(4-(Trifluoromethyl)phenyl)-3-((2,4,4-trimethylpentan-2-yl)amino)-1H-imidazo[1,2-b]pyrazole-7-carboxamide
3-(Tert-butylamino)-2-(3,4-difluorophenyl)-1H-imidazo[1,2-b]pyrazole-7-carboxamide
2-(Pyridin-3-yl)-3-((2,4,4-trimethylpentan-2-yl)amino)-1H-imidazo[1,2-b]pyrazole-7-carboxamide
(E)-3-(Tert-butylamino)-2-(1-phenylprop-1-en-2-yl)-1H-imidazo[1,2-b]pyrazole-7-carboxamide
2-Cyclohexyl-3-((2,4,4-trimethylpentan-2-yl)amino)-1H-imidazo[1,2-b]pyrazole-7-carboxamide
3-(Tert-butylamino)-2-heptyl-1H-imidazo[1,2-b]pyrazole-7-carboxamide
2-(Tert-butyl)-3-(cyclohexylamino)-1H-imidazo[1,2-b]pyrazole-7-carboxamide
2-(Tert-butyl)-3-(tert-butylamino)-1H-imidazo[1,2-b]pyrazole-7-carboxamide
2-(Tert-butyl)-3-((4-methoxyphenyl)amino)-1H-imidazo[1,2-b]pyrazole-7-carboxamide
2-(Tert-butyl)-3-((4-fluorophenyl)amino)-1H-imidazo[1,2-b]pyrazole-7-carboxamide
2-(Tert-butyl)-3-((2,4,4-trimethylpentan-2-yl)amino)-1H-imidazo[1,2-b]pyrazole-7-carbox-amide
2-Cyclopropyl-3-((2,4,4-trimethylpentan-2-yl)amino)-1H-imidazo[1,2-b]pyrazole-7-carboxamide
2-Ethyl-3-((2,4,4-trimethylpentan-2-yl)amino)-1H-imidazo[1,2-b]pyrazole-7-carboxamide
2-Isopropyl-3-((2,4,4-trimethylpentan-2-yl)amino)-1H-imidazo[1,2-b]pyrazole-7-carbox-amide
2-(2-Methylpent-4-en-2-yl)-3-((2,4,4-trimethylpentan-2-yl)amino)-1H-imidazo[1,2-b]pyrazole-7-carboxamide
2-(1-Cyano-3-ethylpentan-3-yl)-3-((2,4,4-trimethylpentan-2-yl)amino)-1H-imidazo[1,2-b]pyrazole-7-carboxamide
Secondary carboxamide and carbothioamide derivatives and pharmaceutically salt thereof
2-(Tert-butyl)-N-methyl-3-((2,4,4-trimethylpentan-2-yl)amino)-1H-imidazo[1,2-b]pyrazole-7-carbox-amide
2-(Tert-butyl)-N-butyl-3-((2,4,4-trimethylpentan-2-yl)amino)-1H-imidazo[1,2-b]pyrazole-7-carbox-amide
N,2-Di-tert-butyl-3-((2,4,4-trimethylpentan-2-yl)amino)-1H-imidazo[1,2-b]pyrazole-7-carboxamide
2-(Tert-butyl)-N-cyclopropyl-3-((2,4,4-trimethylpentan-2-yl)amino)-1H-imidazo[1,2-b]pyrazole-7-carboxamide
2-(Tert-butyl)-N-cyclopentyl-3-((2,4,4-trimethylpentan-2-yl)amino)-1H-imidazo[1,2-b]pyrazole-7-carboxamide
(2-(Tert-butyl)-3-((2,4,4-trimethylpentan-2-yl)amino)-1H-imidazo[1,2-b]pyrazol-7-yl) (piperidin-1-yl)methanone
(2-(Tert-butyl)-3-((2,4,4-trimethylpentan-2-yl)amino)-1H-imidazo[1,2-b]pyrazol-7-yl)(4-phenylpiperazin-1-yl)methanone
N-Benzyl-2-(tert-butyl)-3-((2,4,4-trimethylpentan-2-yl)amino)-1H-imidazo[1,2-b]pyrazole-7-carboxamide
2-(Tert-butyl)-N-phenyl-3-((2,4,4-trimethylpentan-2-yl)amino)-1H-imidazo[1,2-b]pyrazole-7-carboxamide
2-(Tert-butyl)-N-(pyridin-2-yl)-3-((2,4,4-trimethylpentan-2-yl)amino)-1H-imidazo[1,2-b]pyrazole-7-carboxamide
2-(Tert-butyl)-N-(pyridin-3-yl)-3-((2,4,4-trimethylpentan-2-yl)amino)-1H-imidazo[1,2-b]pyrazole-7-carboxamide
2-(Tert-butyl)-N-(pyridin-4-yl)-3-((2,4,4-trimethylpentan-2-yl)amino)-1H-imidazo[1,2-b]pyrazole-7-carboxamide
2-(Tert-butyl)-N-(thiazol-2-yl)-3-((2,4,4-trimethylpentan-2-yl)amino)-1H-imidazo[1,2-b]pyrazole-7-carboxamide
2-(Tert-butyl)-N-(isoxazol-3-yl)-3-((2,4,4-trimethylpentan-2-yl)amino)-1H-imidazo[1,2-b]pyrazole-7-carboxamide
2-(Tert-butyl)-N-(o-tolyl)-3-((2,4,4-trimethylpentan-2-yl)amino)-1H-imidazo[1,2-b]pyrazole-7-carbox-amide
2-(Tert-butyl)-N-(3,5-dimethylphenyl)-3-((2,4,4-trimethylpentan-2-yl)amino)-1H-imidazo[1,2-b]pyrazole-7-carboxamide
2-(Tert-butyl)-N-(4-isopropylphenyl)-3-((2,4,4-trimethylpentan-2-yl)amino)-1H-imidazo[1,2-b]pyrazole-7-carboxamide
2-(Tert-butyl)-N-(4-methoxyphenyl)-3-((2,4,4-trimethylpentan-2-yl)amino)-1H-imidazo[1,2-b]pyrazole-7-carboxamide
2-(Tert-butyl)-N-(2,4-dimethoxyphenyl)-3-((2,4,4-trimethylpentan-2-yl)amino)-1H-imidazo[1,2-b]pyrazole-7-carboxamide
2-(Tert-butyl)-N-(2-(trifluoromethyl)phenyl)-3-((2,4,4-trimethylpentan-2-yl)amino)-1H-imidazo[1,2-b]pyrazole-7-carboxamide
2-(Tert-butyl)-N-(3-(trifluoromethyl)phenyl)-3-((2,4,4-trimethylpentan-2-yl)amino)-1H-imidazo[1,2-b]pyrazole-7-carboxamide
2-(Tert-butyl)-N-(4-(trifluoromethyl)phenyl)-3-((2,4,4-trimethylpentan-2-yl)amino)-1H-imidazo[1,2-b]pyrazole-7-carboxamide
2-(Tert-butyl)-N-(2-fluorophenyl)-3-((2,4,4-trimethylpentan-2-yl)amino)-1H-imidazo[1,2-b]pyrazole-7-carboxamide
2-(Tert-butyl)-N-(3-fluorophenyl)-3-((2,4,4-trimethylpentan-2-yl)amino)-1H-imidazo[1,2-b]pyrazole-7-carboxamide
2-(Tert-butyl)-N-(4-fluorophenyl)-3-((2,4,4-trimethylpentan-2-yl)amino)-1H-imidazo[1,2-b]pyrazole-7-carboxamide
2-(Tert-butyl)-N-(4-chlorophenyl)-3-((2,4,4-trimethylpentan-2-yl)amino)-1H-imidazo[1,2-b]pyrazole-7-carboxamide
N-(4-Bromophenyl)-2-(tert-butyl)-3-((2,4,4-trimethylpentan-2-yl)amino)-1H-imidazo[1,2-b]pyrazole-7-carboxamide
2-(Tert-butyl)-N-(4-nitrophenyl)-3-((2,4,4-trimethylpentan-2-yl)amino)-1H-imidazo[1,2-b]pyrazole-7-carboxamide
2-(Tert-butyl)-N-(4-cyanophenyl)-3-((2,4,4-trimethylpentan-2-yl)amino)-1H-imidazo[1,2-b]pyrazole-7-carboxamide
Ethyl 4-(2-(tert-butyl)-3-((2,4,4-trimethylpentan-2-yl)amino)-1H-imidazo[1,2-b]pyrazole-7-carbox-amido)benzoate
2-(Tert-butyl)-N-(4-(methylthio)phenyl)-3-((2,4,4-trimethylpentan-2-yl)amino)-1H-imidazo[1,2-b]pyrazole-7-carboxamide
2-(Tert-butyl)-N-(4-(dimethylamino)phenyl)-3-((2,4,4-trimethylpentan-2-yl)amino)-1H-imidazo[1,2-b]pyrazole-7-carboxamide
2-(Tert-butyl)-N-(2,4-difluorophenyl)-3-((2,4,4-trimethylpentan-2-yl)amino)-1H-imidazo[1,2-b]pyrazole-7-carboxamide
2-(Tert-butyl)-N-(3,4-difluorophenyl)-3-((2,4,4-trimethylpentan-2-yl)amino)-1H-imidazo[1,2-b]pyrazole-7-carboxamide
2-(Tert-butyl)-N-(4-fluorophenyl)-3-((tert-butyl-2-yl)amino)-1H-imidazo[1,2-b]pyrazole-7-carboxamide
2-(Tert-butyl)-N-(4-fluorophenyl)-3-((cyclohexyl-2-yl)amino)-1H-imidazo[1,2-b]pyrazole-7-carboxamide
2-(Tert-butyl)-3-(tert-butylamino)-N-(4-fluorophenyl)-6-methyl-1H-imidazo[1,2-b]pyrazole-7-carboxamide
2-(Tert-butyl)-3-(tert-butylamino)-N-(4-(trifluoromethyl)phenyl)-1H-imidazo[1,2-b]pyrazole-7-carboxamide
2-(Tert-butyl)-3-(tert-butylamino)-N-(3-(trifluoromethyl)phenyl)-1H-imidazo[1,2-b]pyrazole-7-carboxamide
2-(Tert-butyl)-3-(tert-butylamino)-N-(4-chloro-3-(trifluoromethyl)phenyl)-1H-imidazo[1,2-b]pyrazole-7-carboxamide
2-(Tert-butyl)-3-(tert-butyl(methyl)amino)-N-(4-fluorophenyl)-1H-imidazo[1,2-b]pyrazole-7-carboxamide
2-(Tert-butyl)-3-(tert-butylamino)-N-(5-fluoropyridin-2-yl)-1H-imidazo[1,2-b]pyrazole-7-carboxamide
Methyl 2-((7-((4-fluorophenyl)carbamoyl)-2-(4-(trifluoromethyl)phenyl)-1H-imidazo[1,2-b]pyrazol-3-yl)amino)acetate
Methyl 2-((2-(4-fluoro-3-(trifluoromethyl)phenyl)-7-((4-fluorophenyl)carbamoyl)-1H-imidazo[1,2-b]pyrazol-3-yl)amino)acetate
Methyl 2-((2-(2,4-bis(trifluoromethyl)phenyl)-7-((4-fluorophenyl)carbamoyl)-1H-imidazo[1,2-b]pyrazol-3-yl)amino)acetate
Methyl 2-((2-(3,5-bis(trifluoromethyl)phenyl)-7-((4-fluorophenyl)carbamoyl)-1H-imidazo[1,2-b]pyrazol-3-yl)amino)acetate
2-(Tert-butyl)-N-(4-fluorobenzyl)-3-((2,4,4-trimethylpentan-2-yl)amino)-1H-imidazo[1,2-b]pyrazole-7-carboxamide
2-(Tert-butyl)-N-(5-fluoropyridin-2-yl)-3-((2,4,4-trimethylpentan-2-yl)amino)-1H-imidazo[1,2-b]pyrazole-7-carboxamide
2-(Tert-butyl)-N-(6-fluoropyridin-3-yl)-3-((2,4,4-trimethylpentan-2-yl)amino)-1H-imidazo[1,2-b]pyrazole-7-carboxamide
2-(Tert-butyl)-N-(4-fluorophenyl)-N-methyl-3-((2,4,4-trimethylpentan-2-yl)amino)-1H-imidazo[1,2-b]pyrazole-7-carboxamide
2-(Tert-butyl)-N-(4-fluorophenyl)-6-methyl-3-((2,4,4-trimethylpentan-2-yl)amino)-1H-imidazo[1,2-b]pyrazole-7-carboxamide
2-(Tert-butyl)-N-(4-fluorophenyl)-3-(methyl(2,4,4-trimethylpentan-2-yl)amino)-1H-imidazo[1,2-b]pyrazole-7-carboxamide
2-(Tert-butyl)-N-(6-fluoropyridin-3-yl)-3-(methyl(2,4,4-trimethylpentan-2-yl) amino)-1H-imidazo[1,2-b]pyrazole-7-carboxamide
2-(Tert-butyl)-3-(methyl(2,4,4-trimethylpentan-2-yl)amino)-N-(thiazol-2-yl)-1H-imidazo[1,2-b]pyrazole-7-carboxamide
Methyl 2-((7-((4-fluorophenyl)carbamoyl)-2-(4-(trifluoromethoxy)phenyl)-1H-imidazo[1,2-b]pyrazol-3-yl)amino)acetate
N-(2-(Tert-butyl)-3-(tert-butylamino)-1H-imidazo[1,2-b]pyrazol-7-yl)-4-fluoro-benzamide
3-(Tert-butylamino)-2-cyclopropyl-N-(4-fluorophenyl)-1H-imidazo[1,2-b]pyrazole-7-carbox-amide
N-(4-bromophenyl)-2-(tert-butyl)-3-(tert-butylamino)-1H-imidazo[1,2-b]pyrazole-7-carbox-amide
2-(Tert-butyl)-3-(tert-butylamino)-N-(4-nitrophenyl)-1H-imidazo[1,2-b]pyrazole-7-carboxamide
3-(Tert-butylamino)-2-cyclopropyl-N-(4-nitrophenyl)-1H-imidazo[1,2-b]pyrazole-7-carbox-amide
2-(Tert-butyl)-3-(tert-butylamino)-N-(2-methyl-4-nitrophenyl)-1H-imidazo[1,2-b]pyrazole-7-carboxamide
2-(Tert-butyl)-3-(tert-butylamino)-N-(3-hydroxy-4-nitrophenyl)-1H-imidazo[1,2-b]pyrazole-7-carboxamide
2-(Tert-butyl)-N-(3-hydroxy-4-nitrophenyl)-3-((2,4,4-trimethylpentan-2-yl)amino)-1H-imidazo[1,2-b]pyrazole-7-carboxamide
N-(4-Aminophenyl)-2-(tert-butyl)-3-(tert-butylamino)-1H-imidazo[1,2-b]pyrazole-7-carboxamide
2-(Tert-butyl)-3-(tert-butylamino)-N-(4-(dimethylamino)phenyl)-1H-imidazo[1,2-b]pyrazole-7-carboxamide
N-(4-aminophenyl)-3-(tert-butylamino)-2-cyclopropyl-1H-imidazo[1,2-b]pyrazole-7-carbox-amide
N-(4-Amino-3-hydroxyphenyl)-2-(tert-butyl)-3-(tert-butylamino)-1H-imidazo[1,2-b]pyrazole-7-carboxamide
N-(4-amino-2-methylphenyl)-2-(tert-butyl)-3-(tert-butylamino)-1H-imidazo[1,2-b]pyrazole-7-carboxamide
2-(Tert-butyl)-3-(tert-butylamino)-N-(4-(methylthio)phenyl)-1H-imidazo[1,2-b]pyrazole-7-carboxamide
N-(4-aminophenyl)-2-(tert-butyl)-3-((2,4,4-trimethylpentan-2-yl)amino)-1H-imidazo[1,2-b]pyrazole-7-carboxamide
2-(Tert-butyl)-3-(tert-butylamino)-N-(4-hydroxyphenyl)-1H-imidazo[1,2-b]pyrazole-7-carboxamide
2-(Tert-butyl)-N-(4-hydroxyphenyl)-3-((2,4,4-trimethylpentan-2-yl)amino)-1H-imidazo[1,2-b]pyrazole-7-carboxamide
2-(Tert-butyl)-3-(cyclohexylamino)-N-(4-hydroxyphenyl)-1H-imidazo[1,2-b]pyrazole-7-carboxamide
3-(cyclohexylamino)-N-(4-hydroxyphenyl)-2-(4-(trifluoromethyl)phenyl)-1H-imidazo[1,2-b]pyrazole-7-carboxamide
2-(Tert-butyl)-N-(2-hydroxyphenyl)-3-((2,4,4-trimethylpentan-2-yl)amino)-1H-imidazo[1,2-b]pyrazole-7-carboxamide
2-(tert-butyl)-N-(3-hydroxyphenyl)-3-((2,4,4-trimethylpentan-2-yl)amino)-1H-imidazo[1,2-b]pyrazole-7-carboxamide
2-(Tert-butyl)-N-(4-hydroxy-2-methylphenyl)-3-((2,4,4-trimethylpentan-2-yl) amino)-1H-imidazo[1,2-b]pyrazole-7-carboxamide
2-(Tert-butyl)-N-(4-fluorophenyl)-3-imino-2,3-dihydro-1H-imidazo[1,2-b]pyrazole-7-carboxamide
2-(Tert-butyl)-N-(4-fluorobenzoyl)-3-imino-2,3-dihydro-1H-imidazo[1,2-b]pyrazole-7-carboxamide
2-(Tert-butyl)-3-(tert-butylamino)-N-(4-fluorophenyl)-1H-imidazo[1,2-b]pyrazole-7-carbothioamide
Ethyl 4-(2-(tert-butyl)-3-(tert-butylamino)-1H-imidazo[1,2-b]pyrazole-7-carbox-amido)benzoate
2-(Tert-butyl)-3-(tert-butylamino)-N-(4-(5-((3aS,4S,6aR)-2-oxohexahydro-1H-thieno[3,4-d]imidazol-4-yl)pentanamido)phenyl)-1H-imidazo[1,2-b]pyrazole-7-carboxamide
2-(tert-butyl)-3-(tert-butylamino)-N-(3-(trifluoromethyl)phenyl)-1H-imidazo[1,2-b]pyrazole-7-carboxamide
2-(tert-butyl)-N-(4-hydroxyphenyl)-3-(pentylamino)-1H-imidazo[1,2-b]pyrazole-7-carboxamide
2-(Tert-butyl)-3-(cyclohexylamino)-N-(4-hydroxyphenyl)-1H-imidazo[1,2-b]pyrazole-7-carbox-amide
2-(Tert-butyl)-3-(tert-butylamino)-N-(4-hydroxyphenyl)-1H-imidazo[1,2-b]pyrazole-7-carboxamide
N-(4-aminophenyl)-2-(tert-butyl)-3-((2,4,4-trimethylpentan-2-yl)amino)-1H-imidazo[1,2-b]pyrazole-7-carboxamide
2-(Tert-butyl)-3-(tert-butylamino)-N-(4-(methylthio)phenyl)-1H-imidazo[1,2-b]pyrazole-7-carboxamide
2-(Tert-butyl)-3-(tert-butylamino)-N-(4-(dimethylamino)phenyl)-1H-imidazo[1,2-b]pyrazole-7-carboxamide
2-(Tert-butyl)-N-(4-fluorophenyl)-3-((cyclohexyl-2-yl)amino)-1H-imidazo[1,2-b]pyrazole-7-carboxamide
2-(Tert-butyl)-N-(4-fluorophenyl)-3-((tert-butyl-2-yl)amino)-1H-imidazo[1,2-b]pyrazole-7-carboxamide
2-(Tert-butyl)-N-(isoxazol-3-yl)-3-((2,4,4-trimethylpentan-2-yl)amino)-1H-imidazo[1,2-b]pyrazole-7-carboxamide
2-(Tert-butyl)-N-(thiazol-2-yl)-3-((2,4,4-trimethylpentan-2-yl)amino)-1H-imidazo[1,2-b]pyrazole-7-carboxamide
2-(Tert-butyl)-N-(pyridin-3-yl)-3-((2,4,4-trimethylpentan-2-yl)amino)-1H-imidazo[1,2-b]pyrazole-7-carboxamide
The subject matter of the invention furthermore relates to novel bicyclic imidazo[1,2-b]pyrazole carboxamide derivatives of general formula (V) advantageously of general formula (IV) especially advantageously as listed detailed as follows:
2-(Tert-butyl)-3-(cyclohexylamino)-N-(4-hydroxyphenyl)-1H-imidazo[1,2-b]pyrazole-7-carbox-amide
2-(Tert-butyl)-N-(4-hydroxyphenyl)-3-((2,4,4-trimethylpentan-2-yl)amino)-1H-imidazo[1,2-b]pyrazole-7-carboxamide
2-(Tert-butyl)-3-(tert-butylamino)-N-(4-hydroxyphenyl)-1H-imidazo[1,2-b]pyrazole-7-carboxamide
N-(4-aminophenyl)-2-(tert-butyl)-3-((2,4,4-trimethylpentan-2-yl)amino)-1H-imidazo[1,2-b]pyrazole-7-carboxamide
2-(Tert-butyl)-3-(tert-butylamino)-N-(4-(methylthio)phenyl)-1H-imidazo[1,2-b]pyrazole-7-carboxamide
2-(Tert-butyl)-3-(tert-butylamino)-N-(4-(dimethylamino)phenyl)-1H-imidazo[1,2-b]pyrazole-7-carboxamide
N-(4-Aminophenyl)-2-(tert-butyl)-3-(tert-butylamino)-1H-imidazo[1,2-b]pyrazole-7-carboxamide
2-(Tert-butyl)-N-(4-fluorophenyl)-3-((cyclohexyl-2-yl)amino)-1H-imidazo[1,2-b]pyrazole-7-carboxamide
2-(Tert-butyl)-N-(4-fluorophenyl)-3-((tert-butyl-2-yl)amino)-1H-imidazo[1,2-b]pyrazole-7-carboxamide
2-(Tert-butyl)-N-(4-fluorophenyl)-3-((2,4,4-trimethylpentan-2-yl)amino)-1H-imidazo[1,2-b]pyrazole-7-carboxamide
2-(Tert-butyl)-N-(4-(dimethylamino)phenyl)-3-((2,4,4-trimethylpentan-2-yl)amino)-1H-imidazo[1,2-b]pyrazole-7-carboxamide
2-(Tert-butyl)-N-(2,4-difluorophenyl)-3-((2,4,4-trimethylpentan-2-yl)amino)-1H-imidazo[1,2-b]pyrazole-7-carboxamide
2-(Tert-butyl)-N-(isoxazol-3-yl)-3-((2,4,4-trimethylpentan-2-yl)amino)-1H-imidazo[1,2-b]pyrazole-7-carboxamide
2-(Tert-butyl)-N-(thiazol-2-yl)-3-((2,4,4-trimethylpentan-2-yl)amino)-1H-imidazo[1,2-b]pyrazole-7-carboxamide
2-(Tert-butyl)-N-(pyridin-3-yl)-3-((2,4,4-trimethylpentan-2-yl)amino)-1H-imidazo[1,2-b]pyrazole-7-carboxamide
2-(Tert-butyl)-N-phenyl-3-((2,4,4-trimethylpentan-2-yl)amino)-1H-imidazo[1,2-b]pyrazole-7-carboxamide

The subject matter of the invention furthermore relates to medicinal and/or pharmaceutical compositions comprising the novel bicyclic imidazo[1,2-b]pyrazole carboxamide derivatives disclosed by general formula (V) advantageously of general formula (IV) or (IV′) and further advantageously named and listed specifically as above, and/or pharmaceutically acceptable salts thereof as active agent, which compositions are containing inert, pharmaceutically acceptable, solid or liquid carriers and/or excipients and furthermore relates to the process of formulating the composition comprising the compounds according to the invention.

The subject matter of the invention furthermore relates to

medicinal and/or pharmaceutical compositions, comprising at least one of the subject compounds advantageously solid composition, especially advantageously tablet, inhalation powder or capsule, advantageously semi-solid composition, especially advantageously suppository, or advantageously liquid composition especially advantageously solution for injection.

The subject matter of the invention furthermore relates to a novel process for the preparation of novel bicyclic imidazo[1,2-b]pyrazole carboxamide derivatives described by general formula (V) according to the invention and advantageously named specifically as above, carboxamides advantageously described by general formula (IV) where X represent an O atom, and carbothioamides described by general formula (IV′) where X represent an S atom and pharmaceutically acceptable salts thereof by reacting

a precursor aminopyrazole of general formula (I) where X represents an O atom or of general formula (I′), where X represents an S atom is synthesized from cyanoacetic acid derivative in a three steps manner.

According to the required substitution pattern, arbitrary combinations could be achieved.

The compounds according to the invention are prepared by three component protocol in which aminopyrazoles (I) or (I′) are conducted with the most diverse aldehydes (II) and isonitriles (III), which are commercially available from companies such as Sigma, Alfa Aesar or Fluorochem in the presence of perchloric acid (method A) or trifluoroacetic acid (method B) to form compounds of the general formula (V).

R1 to R4 and X here represent groups of the general formula (V).

The reactions are advantageously accomplished in acetonitril or THF (method A) besides EtOH/water 1:1 (method B) under mild conditions.

Due to the optimized conditions, most of the compounds of general formula (IV) were isolated by simple filtration.

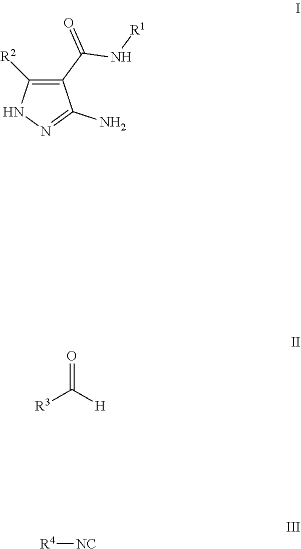

The compounds of the general formula (V) can be converted into their pharmaceutically acceptable salts in a well-known manner to those skilled in the art with physiologically tolerated acids, advantageously hydrochloric acid, acetic acid, oxalic acid, tartaric acid, mandelic acid, fumaric acid, lactic acid, citric acid

### General Procedures (Method A or B) for the Synthesis of Imidazo[1,2-b]Pyrazole Carboxamides of General Formula (IV) or (IV)′

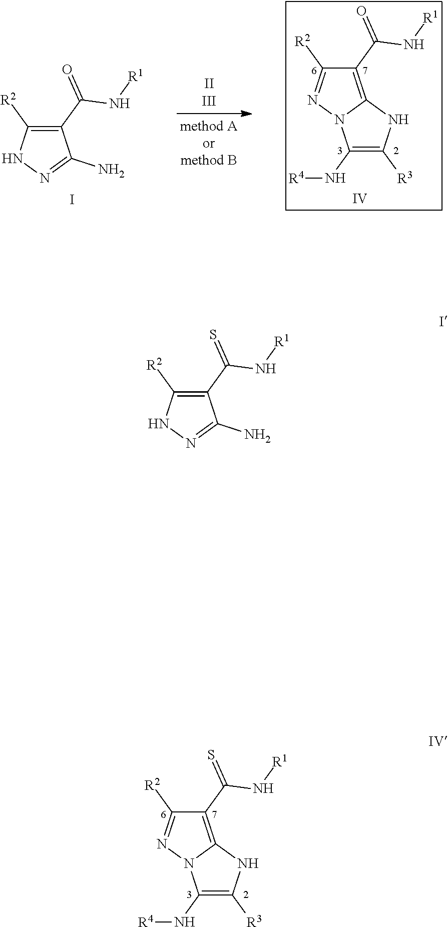

### Method A:

To a suspension of pyrazole of general formulas (I) or (I′) (0.50 mmol) in MeCN or THF (0.5 mL) aldehyde of general formula (II) (0.55 mmol), HClO₄ (20 mol %), and isocyanide of general formula (III) (0.55 mmol) were added and stirred at room temperature for 6 h. Then the crude mixture was purified by filtration followed by washing with cold MeCN or by column chromatography on silica gel (eluent: hexane/EtOAc or chloroform/methanol gradient) to afford pure products of general formulas (IV) or (IV′).

### Method B:

To a suspension of pyrazole of general formulas (I) or (I′) (0.50 mmol) in EtOH/water mixture (1:1, 1 mL) aldehyde of general formula (II) (0.55 mmol), TFA (20 mol %), and isocyanide of general formula (III) (0.55 mmol) were added and stirred at room temperature for 15 minutes.

Then the desired compound of general formulas (IV) or (IV′) was isolated by simple filtration followed by washing with water, then with EtOH.

The subject matter of the invention is furthermore the novel bicyclic imidazo[1,2-b]pyrazole carboxamide derivatives and pharmaceutically acceptable salt thereof according to the invention for use as a medicament for use in the treatment of different diseases, advantageously for treatment of cancer as anticancer agent, as first indication as active ingredient.

The novel bicyclic imidazo[1,2-b] pyrazole carboxamide and carbothioamide derivatives and pharmaceutically acceptable salts thereof according to the invention are advantageously for use in the treatment of solid malignancies, advantageously breast, lung, melanoma, gliomas, and myeloproliferative and myelodysplastic neoplasms, acute myelogenous/myeloid leukemias by the differentiation and subsequent apoptosis of pre-matured myeloid leukemic cells or myeloid-derived suppressor cells.

The novel bicyclic imidazo[1,2-b] pyrazole carboxamide and carbothioamide derivatives according and pharmaceutically acceptable salts thereof to the invention are advantageously for use in the treatment of tumor by eradication of tumor through the differentiation of immature myeloid cells, monocytic and granulocytic myeloid-derived suppressor cells (MDSCs).

The novel bicyclic imidazo[1,2-b] pyrazole carboxamide and carbothioamide derivatives and pharmaceutically acceptable salts thereof according to the invention are advantageously for use in the treatment of tumor by altering cancer cell metabolism as anti-cancer agent, because MDSCs promote tumor growth by several mechanisms including their inherent immunosuppressive activity, promotion of neoangiogenesis, mediation of epithelial-mesenchymal transition.

The novel bicyclic imidazo[1,2-b] pyrazole carboxamide and carbothioamide derivatives and pharmaceutically acceptable salts thereof according to the invention are furthermore advantageously for use in the treatment cancer The pro-tumoral functions of tumor-associated macrophages (TAMs) and MDSCs are further enhanced by their cross-talk offering a myriad of potential anti-cancer therapeutic targets.

The novel bicyclic imidazo[1,2-b] pyrazole carboxamide and carbothioamide derivatives and pharmaceutically acceptable salts thereof according to the invention are furthermore advantageously for use in the treatment for eliminating immature leukemia cells in leukemia, or diminishing tumor-promoting cells in solid tumor microenvironment as anti-cancer agent.

The novel bicyclic imidazo[1,2-b] pyrazole carboxamide and carbothioamide derivatives and pharmaceutically acceptable salts thereof according to the invention are furthermore advantageously for use in the treatment of solid tumor as anti cancer agent by restoration of T-cell immunity, since MDSCs represent immature myeloid cells with inherent immunosuppressive activity differentiation of MDSCs into mature myeloid cells

The novel bicyclic imidazo[1,2-b] pyrazole carboxamide and carbothioamide derivatives and pharmaceutically acceptable salts thereof according to the invention are furthermore advantageously for use in the direct treatment of cells derived from leukemic, as cytotoxic agents.

The novel bicyclic imidazo[1,2-b] pyrazole carboxamide and carbothioamide derivatives and pharmaceutically acceptable salts thereof according to the invention are furthermore advantageously for use in the direct treatment of solid tumor cells as cytotoxic agents.

The novel bicyclic imidazo[1,2-b] pyrazole carboxamide and carbothioamide derivatives and pharmaceutically acceptable salts thereof according to the invention are furthermore advantageously for use in the treatment of cancer cells as anti cancer agent, by inducing differentiation of promyelocytic cells, differentiation induction of various solid cancer cells resulting in apoptosis and cell death by initiating a differentiation followed by subsequent apoptosis of cancer cells.

The novel bicyclic imidazo[1,2-b] pyrazole carboxamide and carbothioamide derivatives and pharmaceutically acceptable salts thereof according to the invention are advantageously for use in the treatment of sepsis by differentiating MDSC.

### History, the State of the Art

The specification of the history, state of the art, concerning the novel imidazo-pyrazole carboxamide derivatives according to the invention, described by general formulas (V), (IV) and (IV′) according to the invention and advantageously named specifically as listed above and pharmaceutically acceptable salts thereof and concerning the medicines suitable for treatment of different diseases and comprising at least one of the subject compounds.

The prior art and patents referred and cited in the present specification hereinafter are all part of the state of the art.

### Chemical Part

The incorporation of the aminopyrazole scaffold into condensed heterocycles has emerged as a powerful strategy for novel anticancer drug development. Numerous pyrazolo[1,5-a]pyrimidines (Hanan et al. 2012; Dwyer et al. 2011; Labroli et al. 2011; Ren et al. 2012; Kosugi et al. 2012; Shaaban et al. 2011), pyrazolo[3,4-d]pyrimidines (Radi et al. 2011a; Dinér et al. 2012; Le Brazidec et al. 2012; Wang et al. 2012; Staben et al. 2010; Soth et al. 2011; Radi et al. 2011b; Yang et al. 2012), pyrazolo[1,5-a][1,3,5]triazines (Popowycz et al. 2009; Nie et al. 2008), pyrazolo[5,1-c][1,2,4]triazoles (Bondock et al. 2012; Hu et al. 2011), and other aminopyrazole-fused bicycles (Bindi et al. 2010; Lukasik et al. 2012; El-borai et al. 2012; Yu et al. 2010; Kim et al. 2011; Raffa et al. 2015; Li et al. 2014) display remarkable cancer-related enzyme inhibitory activities. Despite several synthetic routes are available for the construction of imidazo[1,2-b]pyrazole scaffold, only a limited number of reports focused on their antitumor potential (Sondhi et al. 2002; Terada et al. 1993; Frey et al. 2013; Elleder et al. 2009; Murlykin et al. 2017). In terms of anticancer activity, 3-aminoimidazo[1,2-b]pyrazole-7-carbonitriles 1 were shown to inhibit SYK with IC₅₀ in sub-micromolar range (Zhang et al. 2010), while C7-ethyl ester analogues 2 acted as potent topoisomerase IIα catalytic inhibitors (Baviskar et al. 2011). Imidazo[1,2-b]pyrazole-7-carboxamides 3 were identified as Bruton's tyrosine kinase (BTK) inhibitors (Guo et al. 2014; Wang et al. 2017) and a series of C-7 aminomethylated derivatives 4 was synthesized and showed considerable antitumor activity against five human (A549, Hs683, MCF-7, SKMEL28, U373) and a murine (B16F10) cancer cell types (Grosse et al. 2014).

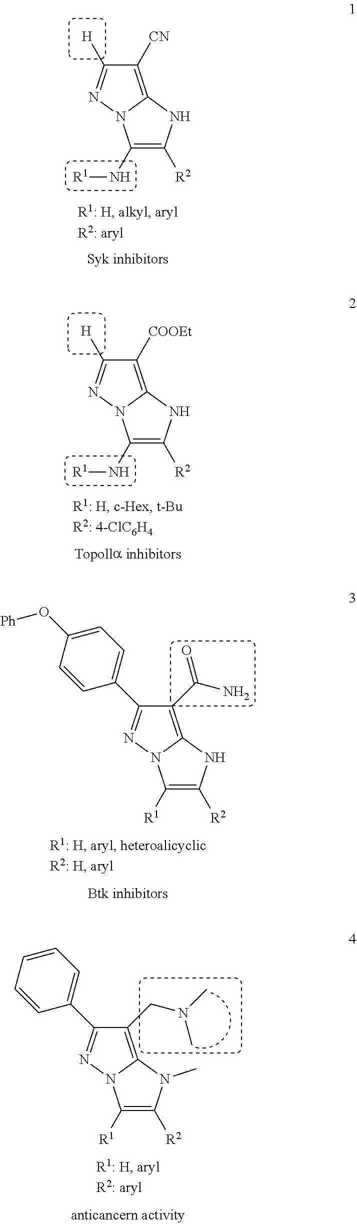

For a construction of an imidazo[1,2-b] based heterocyclic system, the Groebke-Blackburn-Bienaymé three-component reaction could be used, but substrate specific optimization and strategy is required all the time (GBB-3CR; conventional method: assembly of aldehyde, 2-amino-N-heterocycles and isocyanides in the presence of HClO₄ catalyst in MeOH; Demjén et al., 2014; Shaaban et al. 2016; Liu 2015).

### Biological Part

Myeloproliferative neoplasms (MPNs) are diseases of the bone marrow where an excess of cells are produced. These can evolve to myelodysplastic syndromes or myeloid leukemias. MPNs are: Chronic myelogenous leukemia, Chronic neutrophilic leukemia, Polycythemia vera (PV), Primary myelofibrosis (PMF), Essential thrombocythemia (ET), Chronic eosinophilic leukemia (not otherwise specified), Mastocytosis (Vardinan et al. 2009).

In myelodisplastic syndromes (MDS) the cells of bone marrow do not mature into healthy blood cells. MDS are: Refractory anemia (RA), Refractory anemia with ringed sideroblasts (RARS), Refractory cytopenia with multilineage dysplasia (RCMD), Refractory cytopenia with multilineage dysplasia and ringed sideroblasts (RCMD-RS), Refractory anemia with excess blasts (RAEB), Myelodysplastic syndrome, unclassified (MDS-U), MDS associated with isolated del(5q), chronic myelomonocytic leukemia (CMML) and juvenile myelomonocytic leukemia (JMML) (Germing et al. 2013).

Acute myelogenous/myeloid leukemia (AML) originates from myeloid stem cells or myeloid blasts halted in an immature state during haematopoiesis. AML represents a group of heterogeneous forms of myeloid malignancies with diverse genetic abnormalities and different stages of myeloid differentiation. AML is characterized by rapid growth and accumulation of abnormal white blood cells in the bone marrow. AML interfers with the production of normal blood cells. The prototype cells used in our studies are the human cell line, HL-60 which belongs to a sub-type of AML, namely acute promyelocytic leukemia (APL).

Current treatment of myeloproliferative, myelodysplastic diseases or myeloid leukemias are diverse. There is no available curative treatment for any type of MPNs. The aim of therapies in MPNs are to limit the severity of symptoms to avoid thrombohemorrhagic complications, limit anemia and splenomegaly. Low dose aspirin is effective in PV and ET. Tyrosine kinase inhibitors (e.g. imatinib) have improved the prognosis of CML patients (Moen et al. 2007; Tefferi and Pardanani 2015).

The aims of the therapies in the case of MDS are also to diminish the symptoms, improve the quality of life and decrease progression to AML. Allogeneic stem cell (bone marrow) transplantation can be considered under the age of 40 in more severely affected patients. Supporting cares are blood transfusion and the administration of erythropoietin. Chemotherapy for MDSs are performed by the administration of 5-azacytidin, decitabine, lenalidomide (Gangat et al. 2016).

The treatment of AML mostly relies on chemotherapy. Haematopoietic transplantation is suggested mostly in youngers when chemotherapy fails. The aim of the first line treatment called induction phase therapy is complete remission. The second phase is called consolidation therapy to remove any residual disease. During induction therapy cytarabine and anthracycline are given except subtype M3. The acute promyelocytic leukemia (APL, the subtype M3) is treated mainly by all-trans retinoic acid (ATRA). Consolidation chemotherapy eliminates residual malignant cells by a patient-tailored protocol (De Kouchkovsky and Abdul-Hay 2016).

Another pathologic condition of myeloid expansion is the “emergency” granulo-monocytopoiesis in most of the solid malignancies in which, an army of immature myeloid cells leave the bone marrow, called monocytic and granulocytic myeloid-derived suppressor cells (MDSCs) (Strauss et al. 2015). In contrast to AML, MDSCs are not malignant cells, but promote angiogenesis and immunosuppression leading to the progression of cancer. Both in AML and in solid malignancies the differentiation of immature myeloid cells is an already established therapeutic concept (Szebeni et al. 2016).

The most common myeloid infiltrate in solid tumors is composed by myeloid-derived suppressor cells (MDSCs) and tumor-associated macrophages (TAMs) (Szebeni et al. 2017b). In a human phase 1B clinical study 25-dihydroxyvitamin D3 reduced the number of CD34+ immunosuppressive cells, increased HLA-DR expression, elevated plasma IL-12 and IFN-γ level in the blood of HNSSC patients (Lathers et al. 2004). ATRA dramatically reduced the percentage of immature myeloid suppressive cells in the blood of human metastatic renal cell carcinoma patients and improved antigen specific T-cell response (Mirza et al. 2006). The TLR7/8 agonist imidazoquinoline-like molecule, resiquimod treated MDSCs differentiated to F4/80+ macrophages and CD11c+/MHCII+ (I-A^{d+}) dendritic cells exerting potent T-cell stimulatory function (Lee et al. 2014).

MDSCs promote tumor growth by several mechanisms including their inherent immunosuppressive activity, promotion of neoangiogenesis, mediation of epithelial-mesenchymal transition and altering cancer cell metabolism. The pro-tumoral functions of TAMs and MDSCs are further enhanced by their cross-talk offering a myriad of potential anti-cancer therapeutic targets. Since MDSCs represent immature myeloid cells with inherent immunosuppressive activity differentiation of MDSCs into mature myeloid cells thereby restoration of T-cell immunity would be a promising therapeutic strategy (Wesolowski et al. 2013).

Besides inducing differentiation of promyelocytic cells, differentiation induction of various solid cancer cells can also result in apoptosis and cell death. Therefore, the invented compounds could be used not only for eliminating immature leukemia cells in leukemia, or diminishing tumor-promoting cells in solid tumor microenvironment, but the compounds can also act as cytotoxic agents directly on solid tumor cells.

Treating malignant tumor by inducing cell differentiation has been an attractive approach, but clinical development of differentiation-inducing agents to treat malignan solid tumors has been limited to date. Nerve growth factor, all trans retinoic acid, dimethyl sulfoxide, butyric acid, cAMP, vitamin D3, peroxisome proliferator-activated receptorgamma, hexamethylene-bis-acetamide, 12-0-tetradecanoylphorbol 13-acetate, transforming growth factor-beta, and vesnarinone are known to have a differentiation-inducing capability on solid tumors (Kawamata et al, 2006). Moreover some of the differentiation-inducing agents have been used in the clinics for solid tumor, but the therapeutic potential of the differentiation-inducing agents on solid tumor is not strong when compared with that of conventional chemotherapeutic agents. However, because most of the differentiation-inducing agents can potentiate the effect of conventional chemotherapy or radiation therapy, combination of differentiation-inducing agents with conventional chemotherapeutics or radiation therapy might be used in patients with advanced cancer.

The present invention relates to substituted imidazo[1,2-b]pyrazole carboxamides that are able to induce differentiation and subsequent cell death in cancer cell. These compounds could be useful for treatment alone or in combination with known chemotherapeutic agents.

Due to the high mortality of sepsis there is an unmet high medical need for novel therapies. MDSCs can also be targeted in sepsis based on current publications.

It has been published that myeloid-derived cells emerge in septic patients suppressing antigen-driven T-cell proliferation, TH1/Th2 cytokine production contributing to higher prevalence of nosocomial infections.

(Mathias et al. 2017)

Monocytic MDSCs are accumulated in all septic patients whereas granulocytic MDSCs are increased in gram positive case. (Janols et al. 2014)

It has been published that matured MDSCs loose their inherent immunosuppressive phenotype that could solve the dormant state of the antigen specific immune response in sepsis. (McPeak et al. 2017)

MDSCs are immature myeloid cells like our model cell line Hl-60, so based on our previous results XXX compounds may differentiate MDSC as Hl-60 cells have been differentiated upon treatment.

### SUMMARY OF THE INVENTION

1) Chemical Part of the Invention

The present invention relates to novel imidazo[1,2-b]pyrazole carboxamide and carbothioamide derivatives and pharmaceutically acceptable salts thereof, the synthesis thereof, and medicinal and/or pharmaceutical composition comprising these compounds thereof and synthesis thereof,

2) Biological Part of the Invention
The subject compounds are advantageously for use in the treatment of solid malignancies, advantageously breast, lung, melanoma, gliomas, and myeloproliferative and myelodysplastic neoplasms, acute myelogenous/myeloid leukemias by the differentiation and subsequent apoptosis of pre-matured myeloid leukemic cells or myeloid-derived suppressor cells.
The current invention relates to the filed of tumor eradication throught the differentiation of immature myeloid cells, monocytic and granulocytic myeloid-derived suppressor cells (MDSCs). MDSCs promote tumor growth by several mechanisms including their inherent immunosuppressive activity, promotion of neoangiogenesis, mediation of epithelial-mesenchymal transition and altering cancer cell metabolism.
The pro-tumoral functions of tumor-associated macrophages (TAMs) and MDSCs are further enhanced by their cross-talk offering a myriad of potential anti-cancer therapeutic targets. Since MDSCs represent immature myeloid cells with inherent immunosuppressive activity differentiation of MDSCs into mature myeloid cells thereby restoration of T-cell immunity would be a promising therapeutic strategy.
Besides inducing differentiation of promyelocytic cells, differentiation induction of various solid cancer cells can also result in apoptosis and cell death.
It has been presented in vitro that differentiation is initiated and was followed by subsequent apoptosis of cancer cells after treatment with the active compounds. Cells derived from leukemic and solid tumors were readily killed in vitro and in vivo tumor models showed activity in animal tumor models.
Therefore, the invented compounds could be used not only for eliminating immature leukemia cells in leukemia, or diminishing tumor-promoting cells in solid tumor microenvironment, but the compounds can also act as cytotoxic agents directly on solid tumor cells.
The compounds according to the invention are also for use in treatment of sepsis.
MDSCs can namely also be targeted in sepsis.
MDSCs are immature myeloid cells like our model cell line Hl-60, so based on our previous results the compounds according to our invention may differentiate MDSC as Hl-60 cells have been differentiated upon treatment.
The novel biological activity of the compounds according to the invention is backed up by FIGS. 1 to 8 of the specification and of the by Tables 1 to 2 of the examples 102 and 108.

### STATE OF FIGURES

FIG. 1. Compounds described in Example 22, 60 and 83 compromise the viability of HL-60 cells, but human primary fibroblast are resistant to treatment in vitro. Compounds described in Example 22 (FIG. 1. A), 60 (FIG. 1. B) and 83 (FIG. 1. C) dose dependently decreased the viability of HL-60 cells with half-inhibitory concentration (IC₅₀) values of: 940 nM, 210 nM and 50 nM, respectively. Significant decrease in viability was not apparent for human primary fibroblasts in the applied concentration range (1.6 nM-5 μM).

FIG. 2. Compounds described in Example 60 and 83 drive survival pathways as an early response to treatment in HL-60 cells. Using flow cytometry we measured the increase of the percentage of the Bcl-xl^{bright} (A) and pAkt^{bright} cells (B).

FIG. 3. The compound described in Example 83 induces the differentiation of HL-60 promyelocytes. As a proof of cellular differentiation the expression of haematopoietic stem cell markers CD33 and CD34 decreased (A). Matured myeloid cell marker CD11b elevated on the cell surface detected by flow cytometry (B).

FIG. 4. Differentiation of promyelocytic leukemic cells is followed by apoptotic cell death. Differentiation of HL-60 cells was accompanied by apoptosis. We could detect AnnexinV⁺/PI⁻ early and AnnexinV⁺/PI⁺ late apoptotic cell populations after 24 h of treatment.

FIG. 5. Compounds described in Example 60 and 83 induce caspase-3 activation in HL-60 cells. The increased percentage of active caspase-3 positive cells suggested that cell death occurred through the activation of caspase-3 dependent apoptosis.

FIG. 6. The anticancerous effect of the described in Example 83 in live animals: I. mammary carcinoma. In a mammary carcinoma mouse model the intravenous administration of 3 mg/kg of compound 83 reduced the size of the increasing mammary tumour compared to vehicle treated animals.

FIG. 7. The anticancerous effect of the compound described in Example 60 in live animals: II. leukaemia. In a leukaemia mouse model the intravenous administration of 3 mg/kg dose of compound 60 was effective, the treatment increased the LD50 (from day 26 to day 42).

FIG. 8. The anticancerous effect of the compound described in Example 83 in live animals: III. melanoma. In a melanoma mouse model the intravenous administration of 3 mg/kg dose of compound 83 was effective, the treatment increased the LD50 (from day 33 to day 38).

### CHEMICAL EXAMPLES

Concerning the chemical examples of the synthesys of the novel imidazo[1,2-b]pyrazole carboxamide derivatives almost all compounds are of the general formulas of I and IV except the one example 97, where compounds of general formula I′ and IV′ are disclosed, and the prepared compound is a pyrazole carbothioamide.

Therefore only the numbers of the general formula are indicated in the examples with the meaning: “of general formula (number)”.

Accordingly e.g. “reaction conditions (method A): 63 mg

(0.5 mmol) 5-amino-1H-pyrazole-4-carboxamide I”, means:

(0.5 mmol) 5-amino-1H-pyrazole-4-carboxamide of general formula (I).

### Example 1 3-(Tert-butylamino)-2-phenyl-1H-imidazo[1,2-b]pyrazole-7-carboxamide

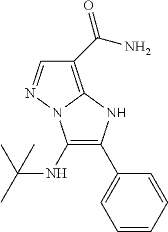 [SMILES: CC(C)(C)Nc1c(-c2ccccc2)[nH]c2c(C(N)=O)cnn12]

Reaction conditions (method A): 63 mg (0.5 mmol) 5-amino-11H-pyrazole-4-carboxamide I; 46 mg (1.1 equiv.) tert-butyl isocyanide III and 58 mg (1.1 equiv.) benzaldehide II in 0.5 mL MeCN, stirring at room temperature for six hours. Flash chromatography purification.

White solid; yield: 69% (method A); m.p. 246-248° C.; ¹H NMR (500 MHz, DMSO-d₆) δ 11.61 (s, 1H), 7.99 (d, J=7.8 Hz, 2H), 7.94 (s, 1H), 7.39 (t, J=7.6 Hz, 2H), 7.26 (t, J=7.4 Hz, 1H), 7.08 (bs, 1H), 6.87 (bs, 1H), 4.04 (bs, 1H), 1.04 (s, 9H). ¹³C NMR (126 MHz, DMSO-d₆) δ 163.9, 142.4, 137.1, 130.6, 128.2, 127.1, 126.7, 124.1, 121.8, 94.0, 54.7, 30.1; ESI-MS (m/z): 298.2 (M+H⁺).

### Example 2 2-Phenyl-3-((2,4,4-trimethylpentan-2-yl)amino)-1H-imidazo[1,2-b]pyrazole-7-carbox-amide

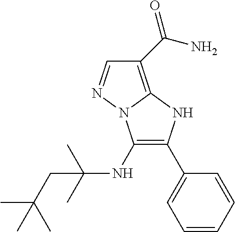 [SMILES: CC(C)(C)CC(C)(C)Nc1c(-c2ccccc2)[nH]c2c(C(N)=O)cnn12]

Reaction conditions (method A): 63 mg (0.5 mmol) 5-amino-1H-pyrazole-4-carboxamide I; 77 mg (1.1 equiv.) 1,1,3,3-tetramethyl butylisocyanide III and 58 mg (1.1 equiv.) benzaldehide II in 0.5 mL MeCN, stirring at room temperature for six hours. Flash chromatography purification with Hexane:Etil-acetate mixture. Pale yellow solid; yield: 51%; m.p. 154-156° C.; ¹H NMR (500 MHz, DMSO-d₆) δ 11.64 (s, 1H), 7.99 (s, 1H), 7.92 (d, J=7.2 Hz, 2H), 7.43 (t, J=7.7 Hz, 2H), 7.32 (t, J=7.4 Hz, 1H), 1.57 (s, 2H), 1.03 (s, 6H), 0.99 (s, 9H). ¹³C NMR (126 MHz, DMSO-d₆) δ 164.3, 142.6, 137.5, 131.0, 128.6, 127.8, 127.6, 125.0, 122.1, 94.4, 59.3, 56.0, 32.1, 31.7, 29.5. ESI-MS (m/z): 354.2 (M+H⁺).

### Example 3 Methyl 2-((7-carbamoyl-2-phenyl-1H-imidazo[1,2-b]pyrazol-3-yl)amino)acetate

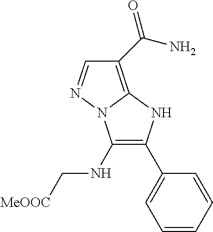 [SMILES: COC(=O)CNc1c(-c2ccccc2)[nH]c2c(C(N)=O)cnn12]

Reaction conditions (method B): 63 mg (0.5 mmol) 5-amino-1H-pyrazole-4-carboxamide I; 54 mg (1.1 equiv.) methyl 2-isocyanoacetate III and 58 mg (1.1 equiv.) benzaldehide II in 0.5 mL EtOH/0.5 mL water, stirring at room temperature for half an hour. Isolation with simple filtration. Gray solid; yield: 46%; m.p. 209-210° C.; ¹H NMR (500 MHz, DMSO-d₆) δ 11.45 (s, 1H), 7.95 (s, 1H), 7.83 (d, J=7.7 Hz, 2H), 7.41 (t, J=7.8 Hz, 2H), 7.25 (t, J=7.4 Hz, 1H), 7.12 (bs, 1H), 6.82 (bs, 1H), 5.59 (s, 1H), 4.22 (s, 2H), 3.55 (s, 3H). ¹³C NMR (126 MHz, DMSO-d₆) δ 171.9, 163.8, 143.0, 137.3, 130.3, 128.5, 126.4, 126.0, 124.2, 115.3, 93.9, 51.5, 46.0. ESI-MS (m/z): 314.1 (M+H⁺).

### Example 4 3-(Cyclohexylamino)-2-phenyl-1H-imidazo[1,2-b]pyrazole-7-carboxamide

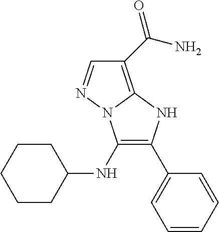 [SMILES: NC(=O)c1cnn2c(NC3CCCCC3)c(-c3ccccc3)[nH]c12]

Reaction conditions (method A): 63 mg (0.5 mmol) 5-amino-1H-pyrazole-4-carboxamide I; 60 mg (1.1 equiv.) cyclohexyl isocyanide III and 58 mg (1.1 equiv.) benzaldehide II in 0.5 mL MeCN, stirring at room temperature for six hours. Flash chromatography purification with Hexane:Etil-acetate mixture. White solid; yield: 51%; m.p. 240-241° C.; ¹H NMR (500 MHz, DMSO-d₆) δ 11.49 (s, 1H), 7.95 (s, 1H), 7.91 (d, J=8.0 Hz, 2H), 7.46-7.35 (m, 2H), 7.28-7.19 (m, 1H), 7.12 (bs, 1H), 6.82 (bs, 1H), 4.53 (bs, 1H), 1.81-1.69 (m, 2H), 1.64-1.55 (m, 2H), 1.50-1.40 (m, 1H), 1.24-1.03 (m, 5H). ¹³C NMR (126 MHz, DMSO-d₆) δ 163.8, 142.8, 137.1, 130.4, 128.4, 126.6, 125.8, 123.5, 119.9, 94.0, 54.2, 33.2, 25.5, 24.3. ESI-MS (m/z): 324.1 (M+H⁺).

### Example 5 3-((4-Methoxyphenyl)amino)-2-phenyl-1H-imidazo[1,2-b]pyrazole-7-carboxamide

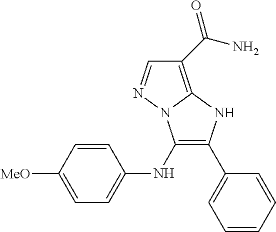 [SMILES: COc1ccc(Nc2c(-c3ccccc3)[nH]c3c(C(N)=O)cnn23)cc1]

Reaction conditions (method A): 63 mg (0.5 mmol) 5-amino-1H-pyrazole-4-carboxamide I; 73 mg (1.1 equiv.) 4-methoxyphenyl isocyanide III and 58 mg (1.1 equiv.) benzaldehide II in 0.5 mL MeCN, stirring at room temperature for six hours. Flash chromatography purification with Hexane:Etil-acetate mixture. Gray solid; yield: 48%; m.p. 229-231° C.; ¹H NMR (500 MHz, DMSO-d₆) δ 11.97 (s, 1H), 7.93 (s, 1H), 7.79 (s, 3H), 7.37 (s, 2H), 7.26 (s, 1H), 6.85 (s, 2H), 6.71 (s, 2H), 6.51 (s, 2H), 3.61 (s, 3H). ¹³C NMR (126 MHz, DMSO-d₆) δ 163.9, 152.3, 142.5, 139.5, 137.7, 129.4, 128.5, 127.5, 126.0, 124.9, 117.6, 114.7, 114.3, 94.7, 55.3. ESI-MS (m/z): 348.2 (M+H⁺).

### Example 6 2-(p-Tolyl)-3-((2,4,4-trimethylpentan-2-yl)amino)-1H-imidazo[1,2-b]pyrazole-7-carbox-amide

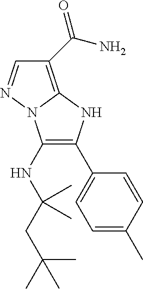 [SMILES: Cc1ccc(-c2[nH]c3c(C(N)=O)cnn3c2NC(C)(C)CC(C)(C)C)cc1]

Reaction conditions (method A): 63 mg (0.5 mmol) 5-amino-1H-pyrazole-4-carboxamide I; 77 mg (1.1 equiv.) 1,1,3,3-tetramethyl butylisocyanide III and 66 mg (1.1 equiv.) p-tolylbenzaldehide II in 0.5 mL MeCN, stirring at room temperature for six hours. Flash chromatography purification with Hexane:Etil-acetate mixture. White solid; yield: 66%; m.p. 218-219° C.; ¹H NMR (500 MHz, DMSO-d₆) δ 11.54 (s, 1H), 7.92 (s, 1H), 7.81 (d, J=7.8 Hz, 2H), 7.20 (d, J=7.7 Hz, 2H), 6.82 (bs, 1H), 3.95 (bs, 1H), 3.49 (bs, 1H), 2.30 (s, 3H), 1.54 (s, 2H), 0.98 (s, 6H), 0.97 (s, 9H). ¹³C NMR (126 MHz, DMSO-d₆) δ 163.9, 142.2, 136.9, 136.5, 128.7, 127.8, 127.1, 124.4, 121.2, 93.9, 58.8, 55.5, 31.7, 31.3, 29.1, 20.9. ESI-MS (m/z): 368.3 (M+H⁺).

### Example 7 2-(4-Methoxyphenyl)-3-((2,4,4-trimethylpentan-2-yl)amino)-1H-imidazo[1,2-b]pyrazole-7-carboxamide

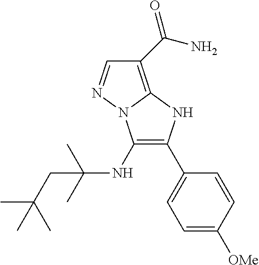 [SMILES: COc1ccc(-c2[nH]c3c(C(N)=O)cnn3c2NC(C)(C)CC(C)(C)C)cc1]

Reaction conditions (method A): 63 mg (0.5 mmol) 5-amino-1H-pyrazole-4-carboxamide I; 77 mg (1.1 equiv.) 1,1,3,3-tetramethyl butylisocyanide III and 75 mg (1.1 equiv.) p-methoxybenzaldehide II in 0.5 mL MeCN, stirring at room temperature for six hours. Flash chromatography purification with Hexane:Etil-acetate mixture. Pale yellow solid; yield: 85%; m.p. 124-125° C.; ¹H NMR (500 MHz, DMSO-d₆) δ 11.43 (s, 1H), 7.91 (s, 1H), 7.86 (d, J=8.3 Hz, 2H), 7.00 (d, J=8.3 Hz, 2H), 6.91-6.74 (bs, 2H), 3.87 (s, 1H), 3.81 (s, 3H), 1.58 (s, 2H), 1.03 (s, 6H), 1.00 (s, 9H). ¹³C NMR (126 MHz, DMSO-d₆) δ 164.4, 159.0, 142.5, 137.3, 129.1, 124.8, 123.6, 121.2, 114.1, 94.4, 59.1, 56.1, 55.7, 32.1, 31.8, 29.6. ESI-MS (m/z): 384.3 (M+H⁺).

### Example 8 4-(7-Carbamoyl-3-((2,4,4-trimethylpentan-2-yl)amino)-1H-imidazo[1,2-b]pyrazol-2-yl)-2-methoxyphenyl acetate

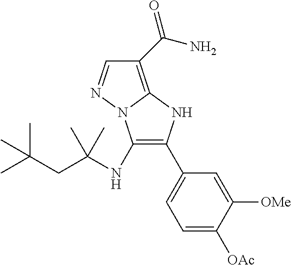 [SMILES: COc1cc(-c2[nH]c3c(C(N)=O)cnn3c2NC(C)(C)CC(C)(C)C)ccc1OC(C)=O]

Reaction conditions (method A): 63 mg (0.5 mmol) 5-amino-1H-pyrazole-4-carboxamide I; 77 mg (1.1 equiv.) 1,1,3,3-tetramethyl butylisocyanide III and 107 mg (1.1 equiv.) disubstituted benzaldehide II in 0.5 mL MeCN, stirring at room temperature for six hours. Flash chromatography purification with Hexane:Etil-acetate mixture. White solid; yield: 50%; m.p. 190-191° C.; ¹H NMR (500 MHz, DMSO-d₆) δ 11.65 (s, 1H), 7.95 (s, 1H), 7.68 (s, 1H), 7.58 (d, J=8.5 Hz, 1H), 7.34-6.97 (m, 1H), 7.09 (d, J=8.3 Hz, 1H), 6.76 (s, 1H), 4.08 (s, 1H), 3.84 (s, 3H), 2.24 (s, 3H), 1.57 (s, 2H), 1.03 (s, 6H), 0.96 (s, 9H). ¹³C NMR (126 MHz, DMSO-d₆) δ 168.5, 164.0, 150.5, 142.1, 138.3, 137.3, 129.4, 123.8, 122.5, 121.6, 119.5, 111.7, 94.0, 58.7, 56.0, 55.6, 31.6, 31.3, 29.1, 20.4. ESI-MS (m/z): 442.3 (M+H⁺).

### Example 9 Methyl 2-((7-carbamoyl-2-(2,4,6-trimethoxyphenyl)-1H-imidazo[1,2-b]pyrazol-3-yl)amino)acetate

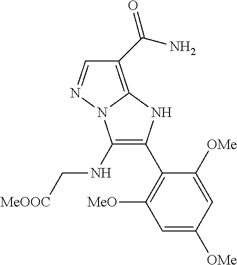 [SMILES: COC(=O)CNc1c(-c2c(OC)cc(OC)cc2OC)[nH]c2c(C(N)=O)cnn12]

Reaction conditions (method B): 63 mg (0.5 mmol) 5-amino-1H-pyrazole-4-carboxamide I; 54 mg (1.1 equiv.) methyl 2-isocyanoacetate III and 108 mg (1.1 equiv.) 2,4,6-trimethoxybenzaldehide II in 0.5 mL EtOH/0.5 mL water, stirring at room temperature for half an hour. Isolation with simple filtration and washing with EtOH. Yellow solid; yield: 74%; m.p. 226-227° C.; ¹H NMR (500 MHz, DMSO-d₆) δ 10.90 (s, 1H), 7.87 (s, 11H), 6.91 (s, 1H), 6.71 (s, 11H), 6.28 (s, 2H), 4.68 (t, J=7.1 Hz, 1H), 3.82 (s, 5H), 3.71 (s, 6H), 3.45 (s, 3H). ¹³C NMR (126 MHz, DMSO-d₆) δ 171.4, 163.9, 161.8, 159.6, 142.1, 136.5, 124.9, 105.8, 99.3, 93.4, 90.7, 55.7, 55.4, 51.3, 45.9. ESI-MS (m/z): 404.1 (M+H⁺).

### Example 10 2-(4-Fluorophenyl)-3-((2,4,4-trimethylpentan-2-yl)amino)-1H-imidazo[1,2-b]pyrazole-7-carboxamide

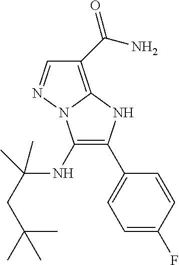 [SMILES: CC(C)(C)CC(C)(C)Nc1c(-c2ccc(F)cc2)[nH]c2c(C(N)=O)cnn12]

Reaction conditions (method A): 63 mg (0.5 mmol) 5-amino-1H-pyrazole-4-carboxamide I; 77 mg (1.1 equiv.) 1,1,3,3-tetramethyl butylisocyanide III and 68 mg (1.1 equiv.) 4-fluorobenzaldehide II in 0.5 mL MeCN, stirring at room temperature for six hours. Flash chromatography purification with Hexane:Etil-acetate mixture. Pale yellow solid; yield: 69%; m.p. 229-230° C.; ¹H NMR (500 MHz, DMSO-d₆) δ 11.66 (s, 1H), 8.08-7.78 (m, 3H), 7.23 (t, J=8.7 Hz, 2H), 6.84 (bs, 2H), 3.97 (s, 1H), 1.52 (s, 2H), 0.98 (s, 6H), 0.95 (s, 9H). ¹³C NMR (126 MHz, DMSO-d₆) δ 164.0, 161.3 (d, J=244.7 Hz), 142.1, 137.2, 129.5 (d, J=8.1 Hz), 127.2, 123.6, 121.4, 115.0 (d, J=21.4 Hz), 94.0, 58.7, 55.5, 31.6, 31.3, 29.0. ESI-MS (m/z): 372.3 (M+H⁺).

### Example 11 Methyl 2-((7-carbamoyl-2-(4-fluorophenyl)-1H-imidazo[1,2-b]pyrazol-3-yl)amino)acetate

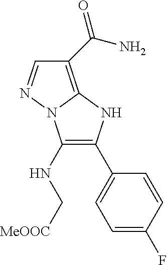 [SMILES: COC(=O)CNc1c(-c2ccc(F)cc2)[nH]c2c(C(N)=O)cnn12]

Reaction conditions (method B): 63 mg (0.5 mmol) 5-amino-1H-pyrazole-4-carboxamide I; 54 mg (1.1 equiv.) methyl 2-isocyanoacetate III and 68 mg (1.1 equiv.) 4-fluorobenzaldehide II in 0.5 mL EtOH/0.5 mL water, stirring at room temperature for half an hour. Isolation with simple filtration and washing with EtOH. White solid; yield: 53%; m.p. 229-230° C.; ¹H NMR (500 MHz, DMSO-d₆) δ 11.49 (s, 1H), 7.95 (s, 1H), 7.88 (dd, J=8.6, 5.3 Hz, 2H), 7.25 (t, J=8.7 Hz, 2H), 7.11 (bs, 1H), 6.79 (bs, 1H), 5.55 (t, J=6.2 Hz, 1H), 4.19 (d, J=6.2 Hz, 2H), 3.55 (s, 3H). ¹³C NMR (126 MHz, DMSO-d₆) δ 171.9, 163.8, 160.9 (d, J=244.5 Hz), 142.8, 137.3, 128.3 (d, J=5.0 Hz), 126.8, 123.8, 115.3 (d, J=21.3 Hz), 115.1, 93.9, 51.5, 46.0. ESI-MS (m/z): 332.1 (M+H⁺).

### Example 12 2-(4-(Trifluoromethyl)phenyl)-3-((2,4,4-trimethylpentan-2-yl)amino)-1H-imidazo[1,2-b]pyrazole-7-carboxamide

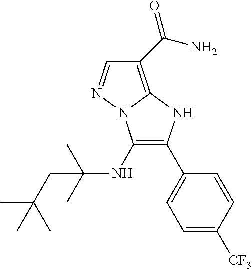 [SMILES: CC(C)(C)CC(C)(C)Nc1c(-c2ccc(C(F)(F)F)cc2)[nH]c2c(C(N)=O)cnn12]

Reaction conditions (method B): 63 mg (0.5 mmol) 5-amino-1H-pyrazole-4-carboxamide I; 77 mg (1.1 equiv.) 1,1,3,3-tetramethyl butylisocyanide III and 96 mg (1.1 equiv.) 4-trifluoromethylbenzaldehide II in 1 mL water/ethanol mixture (1:1), stirring at room temperature for six hours. Flash chromatography purification with Hexane:Etil-acetate mixture. White solid; yield: 69%; m.p. 192-193° C.; ¹H NMR (500 MHz, DMSO-d₆) δ 11.78 (s, 1H), 8.19 (d, J=8.2 Hz, 2H), 8.01 (s, 1H), 7.77 (d, J=8.2 Hz, 2H), 1.60 (s, 2H), 1.06 (s, 6H), 0.99 (s, 9H). ¹³C NMR (126 MHz, DMSO-d₆) δ 164.3, 143.0, 138.3, 135.2, 128.0, 127.7 (q, J=32.0 Hz), 125.4 (q, J=3.5 Hz), 124.8 (q, J=271.7 Hz), 123.4, 94.5, 59.5, 56.0, 32.0, 31.7, 29.5. ESI-MS (m/z): 422.3 (M+H⁺).

### Example 13 3-(Tert-butylamino)-2-(3,4-difluorophenyl)-1H-imidazo[1,2-b]pyrazole-7-carboxamide

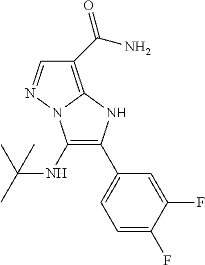 [SMILES: CC(C)(C)Nc1c(-c2ccc(F)c(F)c2)[nH]c2c(C(N)=O)cnn12]

Reaction conditions (method B): 63 mg (0.5 mmol) 5-amino-1H-pyrazole-4-carboxamide I; 77 mg (1.1 equiv.) 1,1,3,3-tetramethyl butylisocyanide III and 77 mg (1.1 equiv.) 3,4-difluorobenzaldehide II in 1 mL water/ethanol mixture (1:1), stirring at room temperature for six hours. Isolation by simple filtration. White solid; yield: 80%; m.p. 256-258° C.; ¹H NMR (500 MHz, DMSO-d₆) δ 11.73 (s, 1H), 8.12 (ddd, J=13.0, 7.8, 2.2 Hz, 1H), 7.97 (s, 1H), 7.92-7.84 (m, 1H), 7.46 (dt, J=10.7, 8.7 Hz, 1H), 7.13 (bs, 1H), 6.85 (bs, 1H), 4.25 (bs, 1H), 1.05 (s, 9H). ¹³C NMR (126 MHz, DMSO-d₆) δ 164.0, 149.2 (dd, J=243.6, 12.7 Hz), 148.3 (dd, J=246.6, 12.4 Hz), 142.3, 137.6, 128.2, 123.6, 122.3, 117.34 (d, J=17.1 Hz), 115.51 (d, J=19.5 Hz), 94.1, 54.8, 30.1. ESI-MS (m/z): 334.4 (M+H⁺).

### Example 14 2-(Pyridin-3-yl)-3-((2,4,4-trimethylpentan-2-yl)amino)-1H-imidazo[1,2-b]pyrazole-7-carboxamide (25)

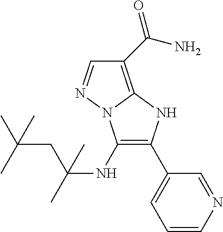 [SMILES: CC(C)(C)CC(C)(C)Nc1c(-c2cccnc2)[nH]c2c(C(N)=O)cnn12]

Reaction conditions (method A): 63 mg (0.5 mmol) 5-amino-1H-pyrazole-4-carboxamide I; 77 mg (1.1 equiv.) 1,1,3,3-tetramethyl butylisocyanide III and 59 mg (1.1 equiv.) 3-pyridine carboxaldehide II in 0.5 mL MeCN, stirring at room temperature for six hours. Flash chromatography purification with Hexane:Etil-acetate mixture. White solid; yield: 82%; m.p. 226-228° C.; ¹H NMR (500 MHz, DMSO-d₆) δ 11.82 (s, 1H), 9.11 (s, 1H), 8.46 (s, 1H), 8.27 (d, J=8.1 Hz, 1H), 7.97 (s, 1H), 7.43 (t, J=6.6 Hz, 1H), 7.14 (bs, 1H), 6.80 (bs, 1H), 4.18 (s, 1H), 1.51 (s, 2H), 0.98 (s, 6H), 0.94 (s, 9H). ¹³C NMR (126 MHz, DMSO-d₆) δ 164.0, 147.9, 147.6, 142.2, 138.0, 134.6, 126.9, 123.2, 122.4, 121.7, 94.1, 58.7, 55.5, 31.6, 31.3, 29.1. ESI-MS (m/z): 355.3 (M+H⁺).

### Example 15 (E)-3-(Tert-butylamino)-2-(1-phenylprop-1-en-2-yl)-1H-imidazo[1,2-b]pyrazole-7-carbox-amide (26)

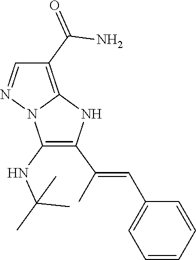 [SMILES: C/C(=C\c1ccccc1)c1[nH]c2c(C(N)=O)cnn2c1NC(C)(C)C]

Reaction conditions (method B): 63 mg (0.5 mmol) 5-amino-1H-pyrazole-4-carboxamide I; 46 mg (1.1 equiv.) tert-butylisocyanide III and 80 mg (1.1 equiv.) α-methylcinnamaldehyde II in 0.5 mL MeCN, stirring at room temperature for six hours. Flash chromatography purification with Hexane:Etil-acetate mixture. White solid; yield: 65%; m.p. 190-191° C.; ¹H NMR (500 MHz, DMSO-d₆) δ 11.33 (s, 1H), 7.92 (s, 1H), 7.40-7.34 (m, 4H), 7.28-7.19 (m, 1H), 7.02 (s, 1H), 6.77 (bs, 1H), 4.15 (bs, 1H), 3.40 (bs, 1H), 2.29 (s, 3H), 1.11 (s, 9H). ¹³C NMR (126 MHz, DMSO-d₆) δ 164.0, 142.2, 137.2, 136.7, 128.9, 128.5, 128.3, 127.2, 127.0, 126.6, 121.9, 93.8, 54.5, 30.0, 16.7. ESI-MS (m/z): 338.2 (M+H⁺).

### Example 16 2-Cyclohexyl-3-((2,4,4-trimethylpentan-2-yl)amino)-1H-imidazo[1,2-b]pyrazole-7-carbox-amide

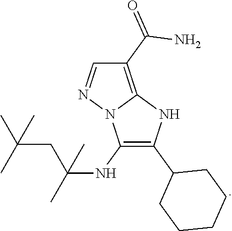 [SMILES: CC(C)(C)CC(C)(C)Nc1c(C2CCCCC2)[nH]c2c(C(N)=O)cnn12]

Reaction conditions (method A): 63 mg (0.5 mmol) 5-amino-1H-pyrazole-4-carboxamide I; 77 mg (1.1 equiv.) 1,1,3,3-tetramethyl butylisocyanide III and 62 mg (1.1 equiv.) cyclohexylaldehide II in 0.5 mL MeCN, stirring at room temperature for six hours. Flash chromatography purification with Hexane:Etil-acetate mixture. White solid; yield: 39%; m.p. 190-192° C.; ¹H NMR (500 MHz, DMSO-d₆) δ 11.15 (s, 1H), 7.81 (s, 1H), 6.96 (bs, 1H), 6.70 (bs, 11H), 3.75 (s, I H), 2.71 (t, J=12.2 Hz, 11H), 1.79-1.68 (m, 4H), 1.67-1.57 (m, 3H), 1.54 (s, 2H), 1.32-1.19 (m, 3H), 1.12 (s, 6H), 1.00 (s, 9H). ¹³C NMR (126 MHz, DMSO-d₆) δ 164.3, 140.8, 136.9, 130.1, 119.3, 93.6, 57.2, 55.1, 33.9, 31.7, 31.5, 31.3, 29.0, 26.4, 25.3. ESI-MS (m/z): 360.3 (M+H⁺).

### Example 17 3-(Tert-butylamino)-2-heptyl-1H-imidazo[1,2-b]pyrazole-7-carboxamide (28)

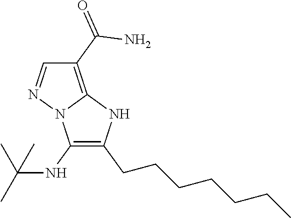 [SMILES: CCCCCCCc1[nH]c2c(C(N)=O)cnn2c1NC(C)(C)C]

Reaction conditions (method A): 63 mg (0.5 mmol) 5-amino-1H-pyrazole-4-carboxamide I; 46 mg (1.1 equiv.) tert-butyl isocyanide III and 70 mg (1.1 equiv.) octanal II in 0.5 mL MeCN, stirring at room temperature for six hours. Flash chromatography purification with Hexane:Etil-acetate mixture. White solid; yield: 44%; m.p. 203-204° C.; ¹H NMR (500 MHz, DMSO-d₆) δ 11.18 (s, 1H), 7.82 (s, 1H), 7.00 (bs, 1H), 6.69 (bs, 1H), 3.84 (bs, 1H), 1.60 (t, J=7.7 Hz, 2H), 1.32-1.18 (m, 10H), 1.09 (s, 9H), 0.88-0.78 (m, 3H). ¹³C NMR (126 MHz, DMSO-d₆) δ 164.4, 140.5, 137.2, 125.5, 120.7, 93.7, 53.6, 31.2, 30.1, 28.8, 28.6, 28.4, 24.2, 22.1, 14.0. ESI-MS (m/z): 320.3 (M+H⁺).

### Example 18 2-(Tert-butyl)-3-(cyclohexylamino)-1H-imidazo[1,2-b]pyrazole-7-carboxamide

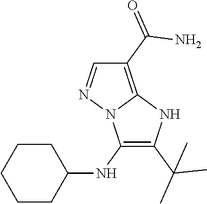 [SMILES: CC(C)(C)c1[nH]c2c(C(N)=O)cnn2c1NC1CCCCC1]

Reaction conditions (method A): 63 mg (0.5 mmol) 5-amino-1H-pyrazole-4-carboxamide I; 60 mg (1.1 equiv.) cyclohexyl isocyanide III and 47 mg (1.1 equiv.) pivalaldehyde II in 0.5 mL MeCN, stirring at room temperature for six hours. Flash chromatography purification with Hexane:Etil-acetate mixture. White solid; yield: 43%; m.p. 148-149° C.; ¹H NMR (500 MHz, DMSO-d₆) δ 10.67 (s, 1H), 7.79 (s, 1H), 6.99 (bs, 11H), 6.63 (bs, 1H), 3.80 (s, 1H), 3.25 (s, 11H), 1.76-1.57 (m, 4H), 1.49 (s, 1H), 1.33 (s, 9H), 1.10 (s, 4H), 0.94 (s, 1H). ¹³C NMR (126 MHz, DMSO-d₆) δ 163.9, 141.8, 135.7, 129.6, 120.6, 93.6, 54.4, 33.3, 31.8, 30.0, 25.6, 24.5. ESI-MS (m/z): 304.3 (M+H⁺).

### Example 19 2-(Tert-butyl)-3-(tert-butylamino)-1H-imidazo[1,2-b]pyrazole-7-carboxamide

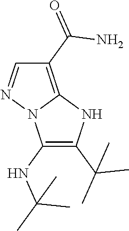 [SMILES: CC(C)(C)Nc1c(C(C)(C)C)[nH]c2c(C(N)=O)cnn12]

Reaction conditions (method B): 63 mg (0.5 mmol) 5-amino-1H-pyrazole-4-carboxamide I; 46 mg (1.1 equiv.) tert-butyl isocyanide III and 47 mg (1.1 equiv.) pivalaldehyde II in 1 mL water/ethanol mixture, stirring at room temperature for 30 minutes. Isolation by simple filtration. White solid; yield: 49%; m.p. 221° C.; ¹H NMR (500 MHz, DMSO-d₆) δ 10.72 (s, 11H), 7.79 (s, 1H), 7.01 (bs, 1H), 6.71 (bs, 1H), 3.59 (s, 1H), 1.36 (s, 9H), 1.17 (s, 9H). ¹³C NMR (126 MHz, DMSO-d₆) δ 164.0, 141.5, 135.9, 132.4, 119.7, 93.5, 52.4, 32.0, 30.7, 30.2. ESI-MS (m/z): 278.2 (M+H⁺).

### Example 20 2-(Tert-butyl)-3-((4-methoxyphenyl)amino)-1H-imidazo[1,2-b]pyrazole-7-carboxamide

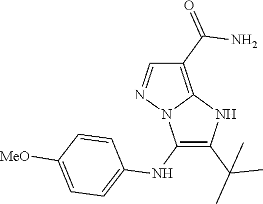 [SMILES: COc1ccc(Nc2c(C(C)(C)C)[nH]c3c(C(N)=O)cnn23)cc1]

Reaction conditions (method B): 63 mg (0.5 mmol) 5-amino-1H-pyrazole-4-carboxamide I; 73 mg (1.1 equiv.) 4-methoxyphenyl isocyanide III and 47 mg (1.1 equiv.) pivalaldehyde II in 1 mL water/ethanol mixture, stirring at room temperature for 30 minutes. Isolation by simple filtration. Pale yellow solid; yield: 41%; m.p. 260-262° C.; ¹H NMR (500 MHz, DMSO-d₆) δ 11.19 (s, 1H), 7.79 (s, 1H), 7.22 (s, 1H), 7.11 (bs, 1H), 6.83 (bs, 1H), 6.68 (d, J=8.3 Hz, 2H), 6.38 (d, J=8.4 Hz, 2H), 3.60 (s, 3H), 1.31 (s, 9H). ¹³C NMR (126 MHz, DMSO-d₆) δ 163.9, 151.8, 141.6, 141.0, 136.5, 133.9, 115.4, 114.6, 113.7, 94.3, 55.3, 32.0, 29.4. ESI-MS (m/z): 328.2 (M+H⁺).

### Example 21 2-(Tert-butyl)-3-((4-fluorophenyl)amino)-1H-imidazo[1,2-b]pyrazole-7-carboxamide

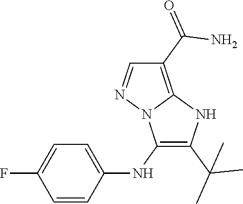 [SMILES: CC(C)(C)c1[nH]c2c(C(N)=O)cnn2c1Nc1ccc(F)cc1]

Reaction conditions (method B): 63 mg (0.5 mmol) 5-amino-1H-pyrazole-4-carboxamide I; 67 mg (1.1 equiv.) 4-fluorophenyl isocyanide III and 47 mg (1.1 equiv.) pivalaldehyde II in 1 mL water/ethanol mixture, stirring at room temperature for 30 minutes. Isolation by simple filtration. Gray solid; yield: 54%; m.p. 249-250° C.; ¹H NMR (500 MHz, DMSO-d₆) δ 11.28 (s, 1H), 7.82 (s, 1H), 7.55 (s, 1H), 7.14 (bs, 1H), 6.91 (t, J=8.6 Hz, 2H), 6.77 (bs, 1H), 6.43 (dd, J=8.5, 4.5 Hz, 2H), 1.32 (s, 9H). ¹³C NMR (126 MHz, DMSO-d₆) δ 163.9, 155.3 (d, J=232.6 Hz), 143.6, 141.7, 136.5, 134.2, 115.4 (d, J=22.3 Hz), 114.8, 113.8 (d, J=7.2 Hz), 94.5, 32.0, 29.4. ESI-MS (m/z): 316.1 (M+H⁺).

### Example 22 2-(Tert-butyl)-3-((2,4,4-trimethylpentan-2-yl)amino)-1H-imidazo[1,2-b]pyrazole-7-carbox-amide

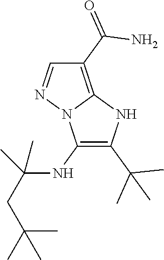 [SMILES: CC(C)(C)CC(C)(C)Nc1c(C(C)(C)C)[nH]c2c(C(N)=O)cnn12]

Reaction conditions (method B): 63 mg (0.5 mmol) 5-amino-1H-pyrazole-4-carboxamide I; 77 mg (1.1 equiv.) 1,1,3,3-tetramethyl butylisocyanide III and 47 mg (1.1 equiv.) pivalaldehyde II in 1 mL water/ethanol mixture, stirring at room temperature for 30 minutes. Isolation by simple filtration. White solid; yield: 54%; m.p. 155-156° C.; ¹H NMR (500 MHz, DMSO-d₆) δ 10.73 (s, 1H), 7.81 (s, 1H), 7.01 (s, 1H), 6.72 (s, 11H), 3.42 (s, 1H), 1.66 (s, 2H), 1.38 (s, 9H), 1.21 (s, 6H), 1.00 (s, 9H). ¹³C NMR (126 MHz, DMSO-d₆) δ 164.0, 141.5, 135.9, 132.4, 119.5, 93.5, 56.5, 56.1, 32.0, 31.8, 31.4, 30.2, 29.6. ESI-MS (m/z): 334.3 (M+H⁺).

### Example 23 2-Cyclopropyl-3-((2,4,4-trimethylpentan-2-yl)amino)-1H-imidazo[1,2-b]pyrazole-7-carboxamide

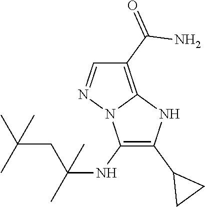 [SMILES: CC(C)(C)CC(C)(C)Nc1c(C2CC2)[nH]c2c(C(N)=O)cnn12]

Reaction conditions (method B): 63 mg (0.5 mmol) 5-amino-1H-pyrazole-4-carboxamide I; 77 mg (1.1 equiv.) 1,1,3,3-tetramethyl butylisocyanide III and 39 mg (1.1 equiv.) cyclopropyl aldehyde II in 1 mL water/ethanol mixture, stirring at room temperature for 30 minutes. Isolation by simple filtration. White solid; yield: 56%; m.p. 205-207° C.; ¹H NMR (500 MHz, DMSO-d₆) δ 10.80 (s, 1H), 7.81 (s, 1H), 6.90 (bs, 1H), 6.73 (bs, 1H), 3.60 (bs, 1H, overlap with water), 2.00-1.92 (m, 1H), 1.59 (s, 2H), 1.14 (s, 6H), 1.02 (s, 9H), 0.91-0.87 (m, 2H), 0.85-0.80 (m, 2H). ¹³C NMR (126 MHz, DMSO-d₆) δ 164.1, 141.1, 136.2, 126.1, 121.5, 93.8, 58.1, 55.3, 31.8, 31.4, 29.2, 7.1, 6.6. ESI-MS (m/z): 318.2 (M+H⁺).

### Example 24 2-Ethyl-3-((2,4,4-trimethylpentan-2-yl)amino)-1H-imidazo[1,2-b]pyrazole-7-carboxamide

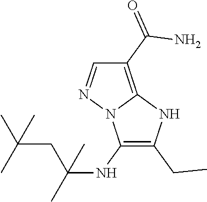 [SMILES: CCc1[nH]c2c(C(N)=O)cnn2c1NC(C)(C)CC(C)(C)C]

Reaction conditions (method B): 63 mg (0.5 mmol) 5-amino-1H-pyrazole-4-carboxamide I; 77 mg (1.1 equiv.) 1,1,3,3-tetramethyl butylisocyanide III and 32 mg (1.1 equiv.) propionaldehyde II in 1 mL water/ethanol mixture, stirring at room temperature for 30 minutes. Isolation by simple filtration. White solid; yield: 51%; m.p. 207-209° C.; ¹H NMR (500 MHz, DMSO-d₆) δ 11.19 (s, 1H), 7.82 (s, 1H), 7.01 (bs, 1H), 6.65 (bs, 1H), 3.68 (s, 1H), 2.55-2.50 (m, 2H), 1.54 (s, 2H), 1.20-1.13 (m, 3H), 1.10 (s, 6H), 1.00 (s, 9H). ¹³C NMR (126 MHz, DMSO-d₆) δ 164.4, 140.5, 137.3, 126.6, 120.1, 93.6, 57.4, 55.2, 31.8, 31.4, 29.1, 17.7, 13.8. ESI-MS (m/z): 306.3 (M+H⁺).

### Example 25 2-Isopropyl-3-((2,4,4-trimethylpentan-2-yl)amino)-1H-imidazo[1,2-b]pyrazole-7-carbox-amide

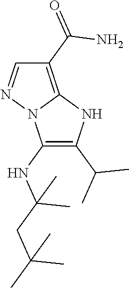 [SMILES: CC(C)c1[nH]c2c(C(N)=O)cnn2c1NC(C)(C)CC(C)(C)C]

Reaction conditions (method B): 63 mg (0.5 mmol) 5-amino-1H-pyrazole-4-carboxamide I; 77 mg (1.1 equiv.) 1,1,3,3-tetramethyl butylisocyanide III and 40 mg (1.1 equiv.) isopropyl aldehyde II in 1 mL water/ethanol mixture, stirring at room temperature for 30 minutes. Isolation by simple filtration. White solid; yield: 42%; m.p. 132-134° C.; ¹H NMR (500 MHz, DMSO-d₆) δ 11.17 (s, 1H), 7.82 (s, 1H), 6.97 (bs, 1H), 6.70 (bs, 1H), 3.70 (bs, 1H), 3.12-3.02 (in, 1H), 1.55 (s, 2H), 1.22 (d, J=7.1 Hz, 6H), 1.12 (s, 6H), 1.00 (s, 9H). ¹³C NMR (126 MHz, DMSO-d₆) δ 164.3, 140.8, 137.2, 130.7, 119.0, 93.7, 57.2, 55.2, 31.8, 31.4, 29.1, 24.0, 21.8. ESI-MS (m/z): 320.4 (M+H⁺).

### Example 26 2-(2-Methylpent-4-en-2-yl)-3-((2,4,4-trimethylpentan-2-yl)amino)-1H-imidazo[1,2-b]pyrazole-7-carboxamide

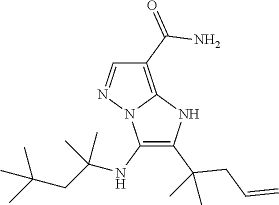 [SMILES: C=CCC(C)(C)c1[nH]c2c(C(N)=O)cnn2c1NC(C)(C)CC(C)(C)C]

Reaction conditions (method B): 63 mg (0.5 mmol) 5-amino-1H-pyrazole-4-carboxamide I; 77 mg (1.1 equiv.) 1,1,3,3-tetramethyl butylisocyanide III and 62 mg (1.1 equiv.) 2,2-dimethylpent-4-enal II in 1 mL water/ethanol mixture, stirring at room temperature for 30 minutes. Isolation by simple filtration. White solid; yield: 45%; m.p. 161-163° C.; ¹H NMR (500 MHz, DMSO-d₆) δ 10.72 (s, 1H), 7.81 (s, 1H), 7.04 (s, 1H), 6.72 (s, 1H), 5.67-5.55 (m, 1H), 5.05-4.89 (m, 2H), 3.43 (s, 1H), 1.65 (s. 2H), 1.35 (s, 6H), 1.22 (s, 6H), 0.99 (s, 9H). ¹³C NMR (126 MHz, DMSO-d₆) δ 164.0, 141.5, 136.0, 135.5, 131.0, 120.5, 117.3, 93.5, 56.4, 56.1, 46.2, 35.2, 31.8, 31.4, 29.6, 27.7. ESI-MS (m/z): 360.3 (M+H⁺).

### Example 27 2-(1-Cyano-3-ethylpentan-3-yl)-3-((2,4,4-trimethylpentan-2-yl)amino)-1H-imidazo[1,2-b]pyrazole-7-carboxamide

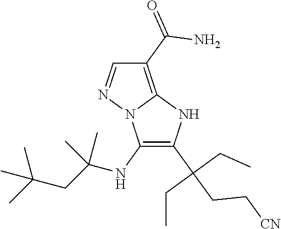 [SMILES: CCC(CC)(CCC#N)c1[nH]c2c(C(N)=O)cnn2c1NC(C)(C)CC(C)(C)C]

Reaction conditions (method B): 63 mg (0.5 mmol) 5-amino-1H-pyrazole-4-carboxamide I; 77 mg (1.1 equiv.) 1,1,3,3-tetramethyl butylisocyanide III and 84 mg (1.1 equiv.) 4-ethyl-4-formylhexanenitrile II in 1 ML water/ethanol mixture, stirring at room temperature for 30 minutes. Isolation by simple filtration. White solid; yield: 35%; m.p. 184-186° C.; ¹H NMR (500 MHz, DMSO-d₆) δ 10.61 (s, 1H), 7.83 (s, 1H), 7.05 (bs, 1H), 6.74 (bs, 1H), 3.56 (d, J=2.1 Hz, 1H), 2.32-2.22 (m, 2H), 2.19-2.13 (m, 2H), 1.76 (q, J=7.2 Hz, 4H), 1.66 (s, 2H), 1.27 (s, 6H), 1.00 (s, 9H), 0.68 (t, J=6.9 Hz, 6H). ¹³C NMR (126 MHz, DMSO-d₆) δ 163.9, 141.7, 136.2, 128.2, 122.8, 121.0, 93.7, 56.2, 56.1, 42.4, 31.8, 31.4, 29.6, 26.6, 11.5, 7.9. ESI-MS (m/z): 401.4 (M+H⁺).

### Example 28 2-(Tert-butyl)-N-methyl-3-((2,4,4-trimethylpentan-2-yl)amino)-1H-imidazo[1,2-b]pyrazole-7-carboxamide

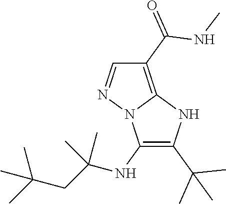 [SMILES: CNC(=O)c1cnn2c(NC(C)(C)CC(C)(C)C)c(C(C)(C)C)[nH]c12]

Reaction conditions (method B): 70 mg (0.5 mmol) 5-amino-N-methyl-1H-pyrazole-4-carboxamide I; 77 mg (1.1 equiv.) 1,1,3,3-tetramethyl butylisocyanide III and 47 mg (1.1 equiv.) pivalaldehyde II in 1 mL water/ethanol mixture, stirring at room temperature for 30 minutes. Isolation by simple filtration. White solid; yield: 31%; m.p. 173-174° C.; ¹H NMR (500 MHz, DMSO-d₆) δ 10.75 (s, 1H), 7.81 (s, 1H), 7.46 (d, J=6.2 Hz, 1H), 3.46 (s, 1H), 2.75 (d, J=4.4 Hz, 3H), 1.68 (s, 2H), 1.41 (s, 9H), 1.23 (s, 6H), 1.03 (s, 9H). ¹³C NMR (126 MHz, DMSO-d₆) δ 163.2, 141.1, 136.2, 132.8, 119.9, 94.0, 56.9, 56.5, 32.5, 32.2, 31.9, 30.7, 30.1, 25.8. ESI-MS (m/z): 348.4 (M+H⁺).

### Example 29 2-(Tert-butyl)-N-butyl-3-((2,4,4-trimethylpentan-2-yl)amino)-1H-imidazo[1,2-b]pyrazole-7-carboxamide

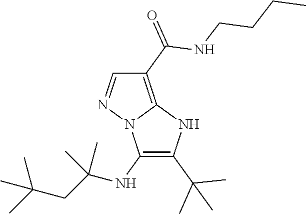 [SMILES: CCCCNC(=O)c1cnn2c(NC(C)(C)CC(C)(C)C)c(C(C)(C)C)[nH]c12]

Reaction conditions (method A): 90 mg (0.5 mmol) 5-amino-N-butyl-1H-pyrazole-4-carboxamide I; 77 mg (1.1 equiv.) 1,1,3,3-tetramethyl butylisocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 43%; m.p. 147-148° C.; ¹H NMR (500 MHz, DMSO-d₆) δ 10.73 (s, 1H), 7.84 (s, 1H), 7.52 (t, J=5.9 Hz, 1H), 3.45 (s, 1H), 3.22 (q, J=6.7 Hz, 2H), 1.68 (s, 2H), 1.48 (q, J=7.2 Hz, 2H), 1.41 (s, 9H), 1.33 (q, J=7.5 Hz, 2H), 1.23 (s, 6H), 1.03 (s, 9H), 0.91 (t, J=7.3 Hz, 3H). ¹³C NMR (126 MHz, DMSO-d₆) δ 162.7, 140.8, 136.5, 132.9, 119.9, 94.2, 56.9, 56.5, 38.4, 32.5, 32.2, 31.9, 30.7, 30.1, 20.1, 14.3. ESI-MS (m/z): 390.3 (M+H⁺).

### Example 30 N,2-Di-tert-butyl-3-((2,4,4-trimethylpentan-2-yl)amino)-1H-imidazo[1,2-b]pyrazole-7-carboxamide

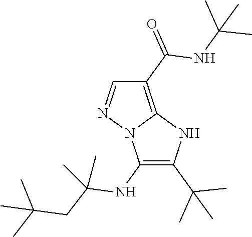 [SMILES: CC(C)(C)CC(C)(C)Nc1c(C(C)(C)C)[nH]c2c(C(=O)NC(C)(C)C)cnn12]

Reaction conditions (method A): 91 mg (0.5 mmol) 5-amino-N-(tert-butyl)-1H-pyrazole-4-carboxamide I; 77 mg (1.1 equiv.) 1,1,3,3-tetramethyl butylisocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 66%; m.p. 177-178° C.; ¹H NMR (500 MHz, DMSO-d₆) δ 10.74 (s, 1H), 7.93 (s, 1H), 6.94 (s, 1H), 3.43 (s, 1H), 1.67 (s, 2H), 1.40 (s, 9H), 1.38 (s, 9H), 1.23 (s, 6H), 1.03 (s, 9H). ¹³C NMR (126 MHz, DMSO-d₆) δ 13C NMR (126 MHz, DMSO) δ 163.1, 140.2, 137.6, 132.9, 119.7, 95.1, 57.0, 56.5, 50.6, 32.5, 32.2, 31.8, 30.7, 30.1, 29.7. ESI-MS (m/z): 390.4 (M+H⁺).

### Example 31 2-(Tert-butyl)-N-cyclopropyl-3-((2,4,4-trimethylpentan-2-yl)amino)-1H-imidazo[1,2-b]pyrazole-7-carboxamide

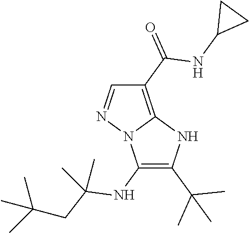 [SMILES: CC(C)(C)CC(C)(C)Nc1c(C(C)(C)C)[nH]c2c(C(=O)NC3CC3)cnn12]

Reaction conditions (method A): 83 mg (0.5 mmol) 5-amino-N-cyclopropyl-1H-pyrazole-4-carboxamide I; 77 mg (1.1 equiv.) 1,1,3,3-tetramethyl butylisocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 57%; m.p. 161° C.; ¹H NMR (500 MHz, DMSO-d₆) δ 10.76 (s, 1H), 7.82 (s, 1H), 7.64-7.55 (m, 1H), 2.75-2.68 (m, 1H), 1.67 (s, 2H), 1.40 (s, 9H), 1.23 (s, 6H), 1.03 (s, 9H), 0.69-0.64 (m, 2H), 0.52-0.48 (m, 2H). ¹³C NMR (126 MHz, DMSO-d₆) δ 163.9, 140.9, 136.6, 132.9, 119.9, 94.0, 56.9, 56.5, 32.5, 32.2, 31.9, 30.7, 30.1, 22.7, 6.5. ESI-MS (m/z): 374.4 (M+H⁺).

### Example 32 2-(Tert-butyl)-N-cyclopentyl-3-((2,4,4-trimethylpentan-2-yl)amino)-1H-imidazo[1,2-b]pyrazole-7-carboxamide

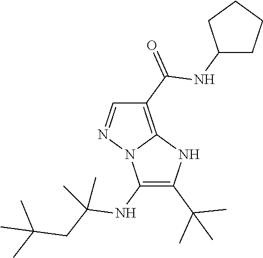 [SMILES: CC(C)(C)CC(C)(C)Nc1c(C(C)(C)C)[nH]c2c(C(=O)NC3CCCC3)cnn12]

Reaction conditions (method A): 97 mg (0.5 mmol) 5-amino-N-cyclopentyl-1H-pyrazole-4-carboxamide I; 77 mg (1.1 equiv.) 1,1,3,3-tetramethyl butylisocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 38%; m.p. 182-183° C.; ¹H NMR (500 MHz, DMSO-d₆) δ 10.75 (s, 1H), 7.90 (s, 1H), 7.40 (s, 1H), 4.19 (s, 1H), 3.44 (s, 1H), 1.89 (s, 2H), 1.69 (s, 4H), 1.51 (s, 4H), 1.41 (s, 9H), 1.24 (s, 6H), 1.04 (s, 9H). ¹³C NMR (126 MHz, DMSO-d₆) δ 164.3, 140.5, 136.6, 133.2, 120.0, 93.4, 57.2, 56.5, 50.9, 32.2, 31.9, 31.2, 31.0, 29.4, 29.1, 23.5. ESI-MS (m/z): 402.4 (M+H⁺).

### Example 33 N-Benzyl-2-(tert-butyl)-3-((2,4,4-trimethylpentan-2-yl)amino)-1H-imidazo[1,2-b]pyrazole-7-carboxamide

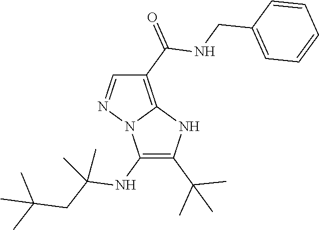 [SMILES: CC(C)(C)CC(C)(C)Nc1c(C(C)(C)C)[nH]c2c(C(=O)NCc3ccccc3)cnn12]

Reaction conditions (method A): 108 mg (0.5 mmol) 5-amino-N-benzyl-1H-pyrazole-4-carboxamide I; 77 mg (1.1 equiv.) 1,1,3,3-tetramethyl butylisocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 49%; m.p. 118-119° C.; ¹H NMR (500 MHz, DMSO-d₆) δ 10.81 (s, 1H), 8.14 (t, J=6.1 Hz, 1H), 7.91 (s, 1H), 7.35-7.30 (m, 4H), 7.28-7.20 (m, 1H), 4.46 (d, J=6.0 Hz, 2H), 3.48 (s, 1H), 1.69 (s, 2H), 1.41 (s, 9H), 1.24 (s, 6H), 1.03 (s, 9H). ¹³C NMR (126 MHz, DMSO-d₆) δ 162.8, 141.1, 141.1, 136.6, 132.9, 128.7, 127.6, 127.0, 120.0, 93.9, 56.9, 56.5, 42.2, 32.5, 32.3, 31.9, 30.7, 30.1. ESI-MS (m/z): 424.3 (M+H⁺).

### Example 34 2-(Tert-butyl)-N-phenyl-3-((2,4,4-trimethylpentan-2-yl)amino)-1H-imidazo[1,2-b]pyrazole-7-carboxamide

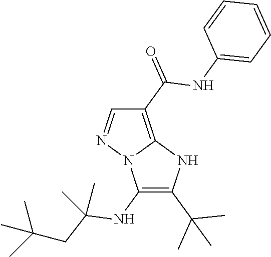 [SMILES: CC(C)(C)CC(C)(C)Nc1c(C(C)(C)C)[nH]c2c(C(=O)Nc3ccccc3)cnn12]

Reaction conditions (method A): 101 mg (0.5 mmol) 5-amino-N-phenyl-1H-pyrazole-4-carboxamide I; 77 mg (1.1 equiv.) 1,1,3,3-tetramethyl butylisocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 55%; m.p. 147° C.; ¹H NMR (500 MHz, DMSO-d₆) δ 11.06 (s, 1H), 9.42 (s, 1H), 8.13 (s, 11H), 7.73 (d, J=7.2 Hz, 2H), 7.31 (t, J=7.9 Hz, 2H), 7.02 (t, J=7.4 Hz, 1H), 3.52 (s, 1H), 1.70 (s, 21H), 1.43 (s, 9H), 1.25 (s, 6H), 1.04 (s, 9H). ¹³C NMR (126 MHz, DMSO-d₆) δ 161.5, 140.8, 140.4, 137.5, 133.3, 129.0, 122.9, 120.1, 120.1, 94.4, 57.0, 56.5, 32.6, 32.2, 31.9, 30.7, 30.1. ESI-MS (m/z): 410.3 (M+H⁺).

### Example 35 2-(Tert-butyl)-N-(pyridin-2-yl)-3-((2,4,4-trimethylpentan-2-yl)amino)-1H-imidazo[1,2-b]pyrazole-7-carboxamide

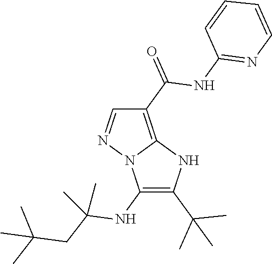 [SMILES: CC(C)(C)CC(C)(C)Nc1c(C(C)(C)C)[nH]c2c(C(=O)Nc3ccccn3)cnn12]

Reaction conditions (method A): 102 mg (0.5 mmol) 5-amino-N-(pyridin-2-yl)-1H-pyrazole-4-carboxamide I; 77 mg (1.1 equiv.) 1,1,3,3-tetramethyl butylisocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 29%; m.p. 144-145° C.; ¹H NMR (500 MHz, DMSO-d₆) δ 11.17 (s, 1H), 10.05 (s, 1H), 8.33 (d, J=4.8 Hz, 1H), 8.29 (s, 1H), 8.22 (d, J=8.5 Hz, 1H), 7.76 (t, J=8.0 Hz, 1H), 7.07 (t, J=6.1 Hz, 1H), 3.53 (s, 1H), 1.69 (s, 2H), 1.43 (s, 9H), 1.25 (s, 6H), 1.04 (s, 9H). ¹³C NMR (126 MHz, DMSO-d₆) δ 161.6, 153.5, 148.1, 141.9, 138.2, 137.2, 133.2, 120.2, 119.0, 114.3, 94.0, 57.0, 56.5, 32.6, 32.3, 31.9, 30.7, 30.1. ESI-MS (m/z): 411.3 (M+H⁺).

### Example 36 2-(Tert-butyl)-N-(pyridin-3-yl)-3-((2,4,4-trimethylpentan-2-yl)amino)-1H-imidazo[1,2-b]pyrazole-7-carboxamide

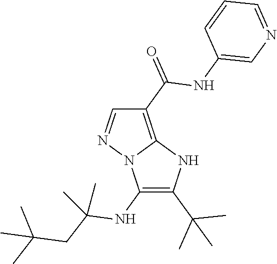 [SMILES: CC(C)(C)CC(C)(C)Nc1c(C(C)(C)C)[nH]c2c(C(=O)Nc3cccnc3)cnn12]

Reaction conditions (method A): 102 mg (0.5 mmol) 5-amino-N-(pyridin-3-yl)-1H-pyrazole-4-carboxamide I; 77 mg (1.1 equiv.) 1,1,3,3-tetramethyl butylisocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 31%; m.p. 186-187° C.; ¹H NMR (500 MHz, DMSO-d₆) δ 11.14 (s, 1H), 9.63 (s, 1H), 8.93 (d, J=2.4 Hz, 1H), 8.23 (dd, J=4.7, 1.5 Hz, 1H), 8.15 (s, 1H), 8.13-8.10 (m, 1H), 7.35 (dd, J=8.3, 4.7 Hz, 1H), 3.54 (s, 1H), 1.70 (s, 2H), 1.43 (s, 9H), 1.25 (s, 6H), 1.04 (s, 9H). ¹³C NMR (126 MHz, DMSO-d₆) S 161.6, 143.8, 141.7, 140.9, 137.5, 137.1, 133.4, 126.8, 123.9, 120.2, 94.0, 57.0, 56.5, 32.6, 32.2, 31.9, 30.7, 30.1. ESI-MS (m/z): 411.3 (M+H⁺).

### Example 37 2-(Tert-butyl)-N-(pyridin-4-yl)-3-((2,4,4-trimethylpentan-2-yl)amino)-1H-imidazo[1,2-b]pyrazole-7-carboxamide

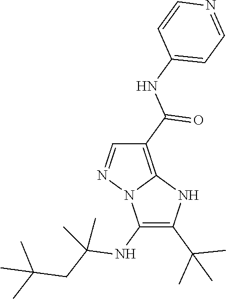 [SMILES: CC(C)(C)CC(C)(C)Nc1c(C(C)(C)C)[nH]c2c(C(=O)Nc3ccncc3)cnn12]

Reaction conditions (method A): 102 mg (0.5 mmol) 5-amino-N-(pyridin-4-yl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. Pale yellow solid; yield: 23%; m.p. 185-186° C.; ¹H NMR (500 MHz, DMSO-d₆) δ 11.24 (s, 1H), 9.80 (s, 1H), 8.40 (d, J=5.5 Hz, 2H), 8.17 (s, 1H), 7.73 (d, J=5.4 Hz, 2H), 3.56 (s, 1H), 1.69 (s, 2H), 1.42 (s, 9H), 1.24 (s, 6H), 1.04 (s, 9H). ¹³C NMR (126 MHz, DMSO-d₆) δ 161.9, 150.5, 147.2, 141.0, 137.6, 133.5, 120.2, 113.7, 94.1, 57.0, 56.4, 32.6, 32.2, 31.9, 30.7, 30.1. ESI-MS (m/z): 411.3 (M+H⁺).

### Example 38 2-(Tert-butyl)-N-(thiazol-2-yl)-3-((2,4,4-trimethylpentan-2-yl)amino)-1H-imidazo[1,2-b]pyrazole-7-carboxamide

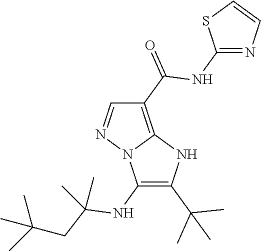 [SMILES: CC(C)(C)CC(C)(C)Nc1c(C(C)(C)C)[nH]c2c(C(=O)Nc3nccs3)cnn12]

Reaction conditions (method A): 105 mg (0.5 mmol) 5-amino-N-(thiazol-2-yl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 44%; m.p. 125-127° C.; ¹H NMR (500 MHz, DMSO-d₆) δ 11.86 (s, 1H), 11.35 (s, 1H), 8.31 (s, 1H), 7.48 (d, J=3.6 Hz, 1H), 7.14 (d, J=3.5 Hz, 1H), 3.55 (s, 1H), 1.69 (s, 2H), 1.42 (s, 9H), 1.24 (s, 6H), 1.04 (s, 9H). ¹³C NMR (126 MHz, DMSO-d₆) δ 160.1, 159.5, 141.8, 137.8, 137.3, 133.4, 120.4, 113.1, 92.5, 57.0, 56.5, 32.6, 32.2, 31.9, 30.7, 30.1. ESI-MS (m/z): 417.3 (M+H⁺).

### Example 39 2-(Tert-butyl)-N-(isoxazol-3-yl)-3-((2,4,4-trimethylpentan-2-yl)amino)-1H-imidazo[1,2-b]pyrazole-7-carboxamide

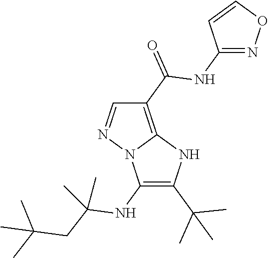 [SMILES: CC(C)(C)CC(C)(C)Nc1c(C(C)(C)C)[nH]c2c(C(=O)Nc3ccon3)cnn12]

Reaction conditions (method A): 108 mg (0.5 mmol) 5-amino-N-(isoxazol-3-yl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. Gray solid; yield: 40%; m.p. 185-186° C.; ¹H NMR (500 MHz, DMSO-d₆) δ 11.21 (s, 1H), 10.66 (s, 1H), 8.77 (d, J=1.8 Hz, 1H), 8.23 (s, 1H), 7.05 (d, J=1.7 Hz, 1H), 3.53 (s, 1H), 1.68 (s, 2H), 1.42 (s, 9H), 1.24 (s, 6H), 1.03 (s, 9H). ¹³C NMR (126 MHz, DMSO-d₆) δ 160.7, 159.9, 158.9, 141.8, 137.3, 133.3, 120.3, 99.9, 93.4, 57.0, 56.5, 32.5, 32.2, 31.9, 30.7, 30.1. ESI-MS (m/z): 401.3 (M+H⁺).

### Example 40 2-(Tert-butyl)-N-(o-tolyl)-3-((2,4,4-trimethylpentan-2-yl)amino)-1H-imidazo[1,2-b]pyrazole-7-carboxamide

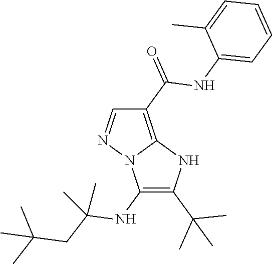 [SMILES: Cc1ccccc1NC(=O)c1cnn2c(NC(C)(C)CC(C)(C)C)c(C(C)(C)C)[nH]c12]

Reaction conditions (method A): 105 mg (0.5 mmol) 5-amino-N-(o-tolyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 30%; m.p. 172° C.; ¹H NMR (500 MHz, DMSO-d₆) δ 10.96 (s, 11H), 8.93 (s, 1H), 8.04 (s, 11H), 7.37 (d, J=7.8 Hz, 1H), 7.26 (d, J=7.4 Hz, 11H), 7.20 (t, J=7.6 Hz, 1H), 7.12 (t, J=7.4 Hz, 1H), 3.53 (s, 1H), 2.26 (s, 3H), 1.71 (s, 2H), 1.42 (s, 9H), 1.26 (s, 6H), 1.05 (s, 9H). ¹³C NMR (126 MHz, DMSO-d₆) δ 161.2, 141.5, 137.3, 136.8, 133.8, 133.1, 130.7, 126.8, 126.3, 125.7, 120.1, 94.0, 57.0, 56.5, 32.5, 32.3, 31.9, 30.7, 30.1, 18.6. ESI-MS (m/z): 424.3 (M+H⁺).

### Example 41 2-(Tert-butyl)-N-(3,5-dimethylphenyl)-3-((2,4,4-trimethylpentan-2-yl)amino)-1H-imidazo[1,2-b]pyrazole-7-carboxamide

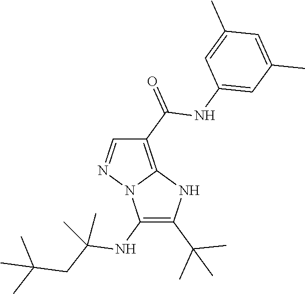 [SMILES: Cc1cc(C)cc(NC(=O)c2cnn3c(NC(C)(C)CC(C)(C)C)c(C(C)(C)C)[nH]c23)c1]

Reaction conditions (method A): 115 mg (0.5 mmol) 5-amino-N-(3,5-dimethylphenyl)-1H-pyrazole-4-carboxamide) I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. Gray solid; yield: 51%; m.p. 180-181° C.; ¹H NMR (500 MHz, DMSO-d₆) δ 11.02 (s, 1H), 9.28 (s, 1H), 8.11 (s, 1H), 7.38 (d, J=1.6 Hz, 2H), 6.67 (s, 1H), 3.51 (s, 1H), 2.25 (s, 6H), 1.70 (s, 2H), 1.42 (s, 9H), 1.25 (s, 6H), 1.04 (s, 9H). ³C NMR (126 MHz, DMSO-d₆) δ 161.4, 140.7, 140.2, 137.8, 137.5, 133.2, 124.4, 120.1, 117.9, 94.5, 57.0, 56.5, 32.5, 32.2, 31.9, 30.7, 30.1, 21.6. ESI-MS (m/z): 438.4 (M+H⁺).

### Example 42 2-(Tert-butyl)-N-(4-isopropylphenyl)-3-((2,4,4-trimethylpentan-2-yl)amino)-1H-imidazo[1,2-b]pyrazole-7-carboxamide

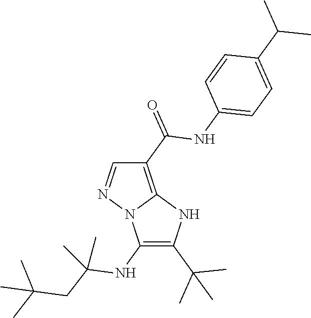 [SMILES: CC(C)c1ccc(NC(=O)c2cnn3c(NC(C)(C)CC(C)(C)C)c(C(C)(C)C)[nH]c23)cc1]

Reaction conditions (method A): 122 mg (0.5 mmol) 5-amino-N-(4-isopropylphenyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 49%; m.p. 155-156° C.; ¹H NMR (500 MHz, DMSO-d₆) δ 11.04 (s, 1H), 9.35 (s, 1H), 8.12 (s, 11H), 7.64 (d, J=8.1 Hz, 2H), 7.18 (d, J=8.2 Hz, 2H), 3.51 (s, 1H), 2.85 (hept, J=7.0 Hz, 111), 1.70 (s, 2H), 1.43 (s, 9H), 1.25 (s, 6H), 1.20 (d, J=6.9 Hz, 6H), 1.04 (s, 9H). ¹³C NMR (126 MHz, DMSO-d₆) δ 161.3, 142.8, 140.8, 138.1, 137.5, 133.2, 126.7, 120.1, 120.0, 94.4, 57.0, 56.5, 33.3, 32.6, 32.2, 31.9, 30.7, 30.1, 24.5. ESI-MS (m/z): 452.4 (M+H⁺).

### Example 43 2-(Tert-butyl)-N-(4-methoxyphenyl)-3-((2,4,4-trimethylpentan-2-yl)amino)-1H-imidazo[1,2-b]pyrazole-7-carboxamide

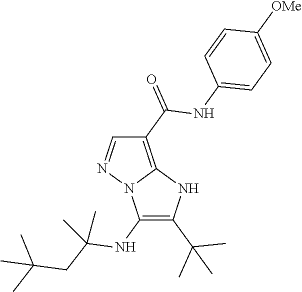 [SMILES: COc1ccc(NC(=O)c2cnn3c(NC(C)(C)CC(C)(C)C)c(C(C)(C)C)[nH]c23)cc1]

Reaction conditions (method A): 116 mg (0.5 mmol) 5-amino-N-(4-methoxyphenyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 59%; m.p. 187-188° C.; ¹H NMR (500 MHz, DMSO-d₆) δ 11.00 (s, 1H), 9.29 (s, 1H), 8.08 (s, 1H), 7.61 (d, J=8.7 Hz, 2H), 6.90 (d, J=8.6 Hz, 2H), 3.51 (s, 1H), 1.70 (s, 2H), 1.43 (s, 9H), 1.25 (s, 6H), 1.04 (s, 9H). ¹³C NMR (126 MHz, DMSO-d₆) δ 161.2, 155.3, 140.8, 137.3, 133.4, 133.2, 121.9, 120.0, 114.1, 94.3, 57.0, 56.5, 55.6, 32.5, 32.2, 31.9, 30.7, 30.1. ESI-MS (m/z): 440.4 (M+H⁺).

### Example 44 2-(Tert-butyl)-N-(2,4-dimethoxyphenyl)-3-((2,4,4-trimethylpentan-2-yl)amino)-1H-imidazo[1,2-b]pyrazole-7-carboxamide

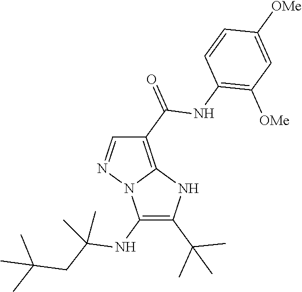 [SMILES: COc1ccc(NC(=O)c2cnn3c(NC(C)(C)CC(C)(C)C)c(C(C)(C)C)[nH]c23)c(OC)c1]

Reaction conditions (method A): 131 mg (0.5 mmol) 5-amino-N-(2,4-dimethoxyphenyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. Light beige solid; yield: 39%; m.p. 127° C.; ¹H NMR (500 MHz, DMSO-d₆) δ 10.95 (s, 1H), 8.51 (s, 1H), 8.00 (s, 1H), 7.50 (d, J=8.7 Hz, 1H), 6.64 (s, 1H), 6.52 (d, J=8.8 Hz, 1H), 3.81 (s, 3H), 3.77 (s, 3H), 3.52 (s, 1H), 1.70 (s, 2H), 1.42 (s, 9H), 1.25 (s, 6H), 1.04 (s, 9H). ¹³C NMR (126 MHz, DMSO-d₆) δ 161.3, 157.7, 153.5, 141.3, 136.7, 133.0, 126.5, 120.7, 120.1, 104.5, 99.2, 94.1, 56.9, 56.5, 56.1, 55.8, 32.5, 32.3, 31.9, 30.7, 30.1. ESI-MS (m/z): 470.4 (M+H).

### Example 45 2-(Tert-butyl)-N-(2-(trifluoromethyl)phenyl)-3-((2,4,4-trimethylpentan-2-yl)amino)-1H-imidazo[1,2-b]pyrazole-7-carboxamide

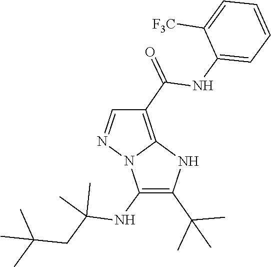 [SMILES: CC(C)(C)CC(C)(C)Nc1c(C(C)(C)C)[nH]c2c(C(=O)Nc3ccccc3C(F)(F)F)cnn12]

Reaction conditions (method A): 135 mg (0.5 mmol) 5-amino-N-(2-(trifluoromethyl)phenyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 34%; m.p. 106° C.; ¹H NMR (500 MHz, DMSO-d₆) δ 11.01 (s, 1H), 9.11 (s, 1H), 8.02 (s, 1H), 7.77 (d, J=7.9 Hz, 1H), 7.71 (t, J=7.6 Hz, 1H), 7.57 (d, J=8.0 Hz, 1H), 7.48 (t, J=7.7 Hz, 1H), 3.54 (s, 1H), 1.71 (s, 2H), 1.42 (s, 9H), 1.26 (s, 6H), 1.05 (s, 9H). ¹³C NMR (126 MHz, DMSO-d₆) δ 161.9, 141.5, 136.9, 136.7, 133.3, 133.1, 131.4, 126.9, 126.8 (q, J=4.6 Hz), 126.2 (q, J=29.1 Hz), 124.3 (d, J=273.6 Hz), 120.2, 93.6, 57.0, 56.5, 32.5, 32.3, 31.9, 30.7, 30.1. ESI-MS (m/z): 478.3 (M+H⁺).

### Example 46 2-(Tert-butyl)-N-(3-(trifluoromethyl)phenyl)-3-((2,4,4-trimethylpentan-2-yl)amino)-1H-imidazo[1,2-b]pyrazole-7-carboxamide

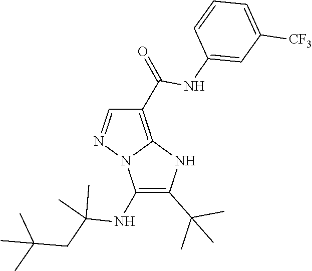 [SMILES: CC(C)(C)CC(C)(C)Nc1c(C(C)(C)C)[nH]c2c(C(=O)Nc3cccc(C(F)(F)F)c3)cnn12]

Reaction conditions (method A): 135 mg (0.5 mmol) 5-amino-N-(3-(trifluoromethyl)phenyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 54%; m.p. 180° C.; ¹H NMR (500 MHz, DMSO-d₆) δ 11.15 (s, 1H), 9.78 (s, 1H, 8.34 (s, 1H), 8.16 (s, 1H), 7.91 (d, J=8.1 Hz, 1H), 7.55 (t, J=8.0 Hz, 1H), 7.35 (d, J=7.6 Hz), 3.54 (s, 1H), 1.70 (s, 2H), 1.43 (s, 9H), 1.25 (s, 6H), 1.04 (s, 9H). ¹³C NMR (126 MHz, DMSO-d₆) δ 161.7, 141.3, 140.7, 137.7, 133.4, 130.1, 129.7 (q, J=31.3 Hz), 124.8 (q, J=272.3 Hz), 123.2, 120.2, 119.0 (q, J=3.7 Hz), 115.9 (q, J=4.3 Hz), 94.1, 57.0, 56.4, 32.6, 32.2, 31.9, 30.7, 30.1. ESI-MS (m/z): 478.4 (M+H⁺).

### Example 47 2-(Tert-butyl)-N-(4-(trifluoromethyl)phenyl)-3-((2,4,4-trimethylpentan-2-yl)amino)-1H-imidazo[1,2-b]pyrazole-7-carboxamide

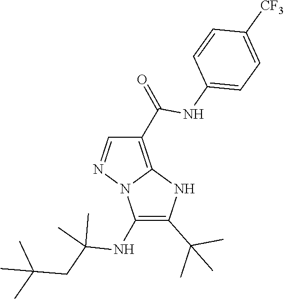 [SMILES: CC(C)(C)CC(C)(C)Nc1c(C(C)(C)C)[nH]c2c(C(=O)Nc3ccc(C(F)(F)F)cc3)cnn12]

Reaction conditions (method A): 135 mg (0.5 mmol) 5-amino-N-(4-(trifluoromethyl)phenyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 39%; m.p. 155-156° C.; ¹H NMR (500 MHz, DMSO-d₆) δ 11.17 (s, 1H), 9.78 (s, 1H), 8.17 (s, 1H), 7.97 (d, J=8.5 Hz, 2H), 7.67 (d, J=8.5 Hz, 2H), 3.54 (s, 1H), 1.70 (s, 2H), 1.43 (s, 9H), 1.25 (s, 6H), 1.04 (s, 9H). ¹³C NMR (126 MHz, DMSO-d₆) δ 161.6, 144.2, 140.9, 137.6, 133.4, 126.3 (d, J=3.5 Hz), 125.1 (q, J=271.0 Hz), 122.7 (q, J=31.7 Hz), 120.2, 119.6, 94.2, 57.0, 56.5, 32.6, 32.2, 31.9, 30.7, 30.1. ESI-MS (m/z): 478.4 (M+H⁺).

### Example 48 2-(Tert-butyl)-N-(2-fluorophenyl)-3-((2,4,4-trimethylpentan-2-yl)amino)-1H-imidazo[1,2-b]pyrazole-7-carboxamide

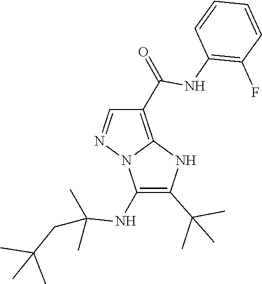 [SMILES: CC(C)(C)CC(C)(C)Nc1c(C(C)(C)C)[nH]c2c(C(=O)Nc3ccccc3F)cnn12]

Reaction conditions (method A): 110 mg (0.5 mmol) 5-amino-N-(2-fluorophenyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 39%; m.p. 153-154° C.; ¹H NMR (500 MHz, DMSO-d₆) δ 10.91 (s, 1H), 9.08 (s, 1H), 8.09 (s, 1H), 7.74-7.65 (m, 1H), 7.29-7.22 (m, 1H), 7.22-7.16 (m, 2H), 3.46 (s, 1H), 1.73 (s, 2H), 1.44 (s, 9H), 1.27 (s, 6H), 1.06 (s, 9H). ¹³C NMR (126 MHz, DMSO-d₆) δ 161.2, 155.8 (d, J=245.3 Hz), 141.6, 137.1, 133.2, 127.0, 126.9, 126.0 (d, J=7.5 Hz), 124.5 (d, J=3.3 Hz), 120.3, 116.0 (d, J=20.2 Hz), 93.8, 57.1, 56.6, 32.5, 32.2, 31.8, 30.7, 30.1. ESI-MS (m/z): 428.3 (M+H⁺).

### Example 49 2-(Tert-butyl)-N-(3-fluorophenyl)-3-((2,4,4-trimethylpentan-2-yl)amino)-1H-imidazo[1,2-b]pyrazole-7-carboxamide

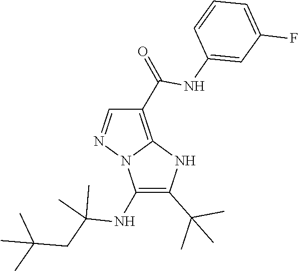 [SMILES: CC(C)(C)CC(C)(C)Nc1c(C(C)(C)C)[nH]c2c(C(=O)Nc3cccc(F)c3)cnn12]

Reaction conditions (method A): 110 mg (0.5 mmol) 5-amino-N-(3-fluorophenyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 47%; m.p. 159° C.; ¹H NMR (500 MHz, DMSO-d₆) δ 11.13 (s, 1H), 9.62 (s, 1H), 8.14 (s, 1H), 7.78 (dt, J=12.3, 2.4 Hz, 1H), 7.56-7.42 (m, 1H), 7.39-7.26 (m, 1H), 6.83 (td, J=8.4, 2.6 Hz, 1H), 3.53 (s, 1H), 1.70 (s, 2H), 1.43 (s, 9H), 1.25 (s, 6H), 1.04 (s, 9H). ¹³C NMR (126 MHz, DMSO-d₆) δ 163.6, 162.6 (d, J=240.2 Hz), 142.3 (d, J=11.2 Hz), 140.8, 137.6, 133.3, 130.5 (d, J=9.7 Hz), 120.1, 115.5, 109.1 (d, J=21.2 Hz), 106.5 (d, J=26.5 Hz), 94.2, 57.0, 56.5, 32.6, 32.2, 31.9, 30.7, 30.1. ESI-MS (m/z): 428.4 (M+H⁺).

### Example 50 2-(Tert-butyl)-N-(4-fluorophenyl)-3-((2,4,4-trimethylpentan-2-yl)amino)-1H-imidazo[1,2-b]pyrazole-7-carboxamide

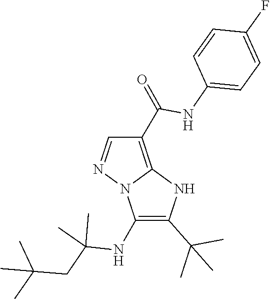 [SMILES: CC(C)(C)CC(C)(C)Nc1c(C(C)(C)C)[nH]c2c(C(=O)Nc3ccc(F)cc3)cnn12]

Reaction conditions (method A): 110 mg (0.5 mmol) 5-amino-N-(4-fluorophenyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 49%; m.p. 188° C.; ¹H NMR (500 MHz, DMSO-d₆) δ 11.06 (s, 1H), 9.48 (s, 1H), 8.10 (s, 1H), 7.80-7.67 (m, 2H), 7.15 (t, J=8.9 Hz, 2H), 3.52 (s, 1H), 1.70 (s, 2H), 1.42 (s, 9H), 1.25 (s, 6H), 1.04 (s, 9H). ¹³C NMR (126 MHz, DMSO-d₆) δ 161.4, 158.1 (d, J=238.7 Hz), 140.8, 137.4, 136.7, 133.3, 121.8 (d, J=7.9 Hz), 120.1, 115.5 (d, J=22.0 Hz), 94.2, 57.0, 56.5, 32.6, 32.2, 31.9, 30.7, 30.1. ESI-MS (m/z): 428.4 (M+H⁺).

### Example 512-(Tert-butyl)-N-(4-chlorophenyl)-3-((2,4,4-trimethylpentan-2-yl)amino)-1H-imidazo[1,2-b]pyrazole-7-carboxamide

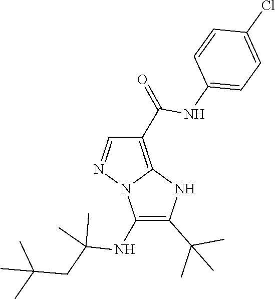 [SMILES: CC(C)(C)CC(C)(C)Nc1c(C(C)(C)C)[nH]c2c(C(=O)Nc3ccc(Cl)cc3)cnn12]

Reaction conditions (method A): 118 mg (0.5 mmol) 5-amino-N-(4-chlorophenyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 50%; m.p. 188° C.; ¹H NMR (500 MHz, DMSO-d₆) δ 11.09 (s, 1H), 9.55 (s, 1H), 8.12 (s, 1H), 7.77 (d, J=8.8 Hz, 2H), 7.36 (d, J=8.7 Hz, 2H), 3.52 (s, 1H), 1.70 (s, 2H), 1.42 (s, 9H), 1.25 (s, 6H), 1.04 (s, 9H). ¹³C NMR (126 MHz, DMSO-d₆) δ 161.4, 140.8, 139.4, 137.5, 133.3, 128.9, 126.4, 121.5, 120.1, 94.19, 57.0, 56.5, 32.6, 32.2, 31.9, 30.7, 30.1. ESI-MS (m/z): 444.3 and 446.3 (M+H⁺).

### Example 52 N-(4-Bromophenyl)-2-(tert-butyl)-3-((2,4,4-trimethylpentan-2-yl)amino)-1H-imidazo[1,2-b]pyrazole-7-carboxamide

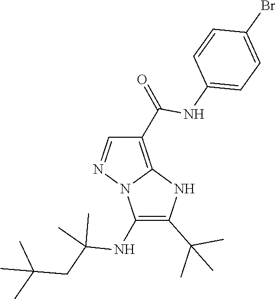 [SMILES: CC(C)(C)CC(C)(C)Nc1c(C(C)(C)C)[nH]c2c(C(=O)Nc3ccc(Br)cc3)cnn12]

Reaction conditions (method A): 140 mg (0.5 mmol) 5-amino-N-(4-bromophenyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. Pale yellow solid; yield: 42%; m.p. 153-154° C.; ¹H NMR (500 MHz, DMSO-d₆) δ 11.10 (s, 1H), 9.55 (s, 1H), 8.12 (s, 1H), 7.72 (d, J=8.5 Hz, 2H), 7.49 (d, J=8.5 Hz, 2H), 3.53 (s, 1H), 1.70 (s, 2H), 1.42 (s, 9H), 1.25 (s, 6H), 1.04 (s, 9H). ¹³C NMR (126 MHz, DMSO-d₆) δ 161.4, 140.8, 139.8, 137.5, 133.3, 131.8, 121.9, 120.1, 114.3, 94.2, 57.0, 56.5, 32.6, 32.2, 31.9, 30.7, 30.1. ESI-MS (m/z): 488.2 and 490.3 (M+H⁺).

### Example 53 2-(Tert-butyl)-N-(4-nitrophenyl)-3-((2,4,4-trimethylpentan-2-yl)amino)-1H-imidazo[1,2-b]pyrazole-7-carboxamide

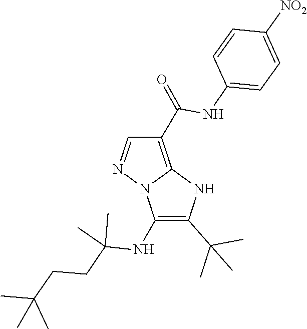 [SMILES: CC(C)(C)CCC(C)(C)Nc1c(C(C)(C)C)[nH]c2c(C(=O)Nc3ccc([N+](=O)[O-])cc3)cnn12]

Reaction conditions (method A): 124 mg (0.5 mmol) 5-amino-N-(4-nitrophenyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. Yellow solid; yield: 38%; m.p. 199° C.; ¹H NMR (500 MHz, DMSO-d₆) δ 11.27 (s, 1H), 10.06 (s, 1H), 8.24 (d, J=9.2 Hz, 2H), 8.19 (s, 1H), 8.01 (d, J=9.1 Hz, 2H), 3.57 (s, 1H), 1.69 (s, 2H), 1.43 (s, 9H), 1.25 (s, 6H), 1.04 (s, 9H). ¹³C NMR (126 MHz, DMSO-d₆) δ 161.6, 147.1, 141.9, 141.2, 137.7, 133.5, 125.2, 120.4, 119.2, 94.1, 57.1, 56.5, 32.6, 32.2, 31.8, 30.7, 30.1. ESI-MS (m/z): 455.3 (M+H⁺).

### Example 54 2-(Tert-butyl)-N-(4-cyanophenyl)-3-((2,4,4-trimethylpentan-2-yl)amino)-1H-imidazo[1,2-b]pyrazole-7-carboxamide

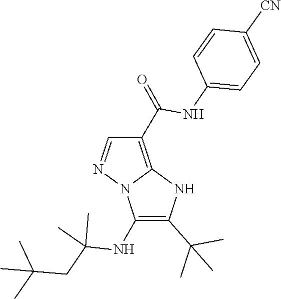 [SMILES: CC(C)(C)CC(C)(C)Nc1c(C(C)(C)C)[nH]c2c(C(=O)Nc3ccc(C#N)cc3)cnn12]

Reaction conditions (method A): 113 mg (0.5 mmol) 5-amino-N-(4-cyanophenyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 48%; m.p. 199° C.; ¹H NMR (500 MHz, DMSO-d₆) δ 11.20 (s, 1H), 9.85 (s, 1H), 8.16 (s, 1H), 7.94 (d, J=8.8 Hz, 2H), 7.76 (d, J=8.7 Hz, 2H), 3.55 (s, 1H), 1.70 (s, 2H), 1.43 (s, 9H), 1.25 (s, 6H), 1.04 (s, 9H). ¹³C NMR (126 MHz, DMSO-d₆) δ 161.6, 144.9, 141.0, 137.6, 133.5, 133.4, 120.2, 119.8, 119.7, 104.2, 94.1, 57.0, 56.5, 32.6, 32.2, 31.9, 30.7, 30.1. ESI-MS (m/z): 435.3 (M+H⁺).

### Example 55 Ethyl 4-(2-(tert-butyl)-3-((2,4,4-trimethylpentan-2-yl)amino)-1H-imidazo[1,2-b]pyrazole-7-carbox-amido)benzoate

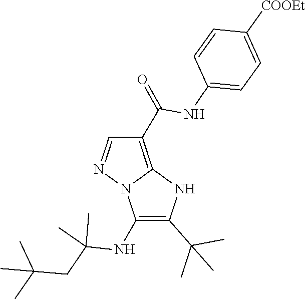 [SMILES: CCOC(=O)c1ccc(NC(=O)c2cnn3c(NC(C)(C)CC(C)(C)C)c(C(C)(C)C)[nH]c23)cc1]

Reaction conditions (method A): 137 mg (0.5 mmol) ethyl 4-(5-amino-1H-pyrazole-4-carboxamido) benzoate I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 60%; m.p. 127-128° C.; ¹H NMR (500 MHz, DMSO-d₆) δ 11.17 (s, 1H), 9.78 (s, 1H), 8.17 (s, 1H), 7.97-7.83 (m, 4H), 4.29 (q, J=7.1 Hz, 2H), 3.54 (s, 1H), 1.70 (s, 2H), 1.43 (s, 9H), 1.32 (t, J=7.1 Hz, 3H), 1.25 (s, 6H), 1.04 (s, 9H). ¹³C NMR (126 MHz, DMSO-d₆) δ 165.9, 161.6, 145.1, 140.9, 137.6, 133.4, 130.5, 123.6, 120.2, 119.1, 94.3, 60.8, 57.0, 56.5, 32.6, 32.2, 31.9, 30.7, 30.1, 14.7. ESI-MS (m/z): 482.4 (M+H⁺).

### Example 56 2-(Tert-butyl)-N-(4-(methylthio)phenyl)-3-((2,4,4-trimethylpentan-2-yl)amino)-1H-imidazo[1,2-b]pyrazole-7-carboxamide

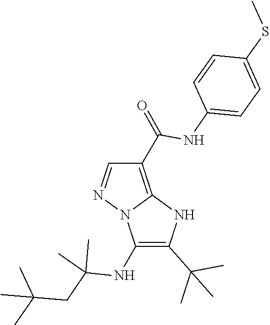 [SMILES: CSc1ccc(NC(=O)c2cnn3c(NC(C)(C)CC(C)(C)C)c(C(C)(C)C)[nH]c23)cc1]

Reaction conditions (method A): 124 mg (0.5 mmol) 5-amino-N-(4-(methylthio)phenyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 28%; m.p. 169-170° C.; ¹H NMR (500 MHz, DMSO-d₆) δ 11.06 (s, 1H), 9.44 (s, 1H), 8.11 (s, 1H), 7.70 (d, J=8.3 Hz, 2H), 7.25 (d, J=8.3 Hz, 2H), 3.52 (s, 1H), 2.46 (s, 3H), 1.70 (s, 2H), 1.43 (s, 9H), 1.25 (s, 6H), 1.04 (s, 9H). ¹³C NMR (126 MHz, DMSO-d₆) δ 161.4, 140.8, 138.0, 137.5, 133.3, 131.1, 127.7, 120.8, 120.1, 94.3, 57.0, 56.5, 32.6, 32.2, 31.9, 30.7, 30.1, 16.2. ESI-MS (m/z): 456.3 (M+H⁺).

### Example 57 2-(Tert-butyl)-N-(4-(dimethylamino)phenyl)-3-((2,4,4-trimethylpentan-2-yl)amino)-1H-imidazo[1,2-b]pyrazole-7-carboxamide

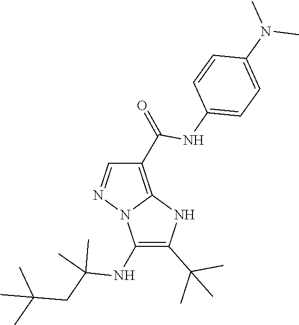 [SMILES: CN(C)c1ccc(NC(=O)c2cnn3c(NC(C)(C)CC(C)(C)C)c(C(C)(C)C)[nH]c23)cc1]

Reaction conditions (method A): 123 mg (0.5 mmol) 5-amino-N-(4-(dimethylamino)phenyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. Gray solid; yield: 37%; m.p. 176-177° C.; ¹H NMR (500 MHz, DMSO-d₆) δ 10.96 (s, 1H), 9.16 (s, 1H), 8.06 (s, 1H), 7.51 (d, J=8.6 Hz, 2H), 6.72 (d, J=8.5 Hz, 2H), 3.50 (s, 1H), 3.35 (s, 6H), 1.70 (s, 2H), 1.43 (s, 9H), 1.25 (s, 6H), 1.04 (s, 9H). ¹³C NMR (126 MHz, DMSO-d₆) δ 161.1, 147.2, 140.8, 137.2, 133.1, 130.1, 121.9, 120.0, 113.2, 94.5, 57.0, 56.5, 41.1, 32.5, 32.3, 31.9, 30.7, 30.1. APCI-MS (m/z): 453.3 (M+H⁺).

### Example 58 2-(Tert-butyl)-N-(2,4-difluorophenyl)-3-((2,4,4-trimethylpentan-2-yl)amino)-1H-imidazo[1,2-b]pyrazole-7-carboxamide

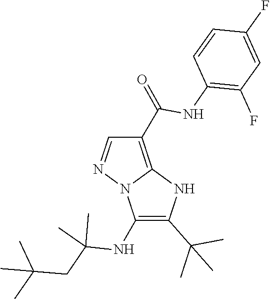 [SMILES: CC(C)(C)CC(C)(C)Nc1c(C(C)(C)C)[nH]c2c(C(=O)Nc3ccc(F)cc3F)cnn12]

Reaction conditions (method A): 119 mg (0.5 mmol) 5-amino-N-(2,4-difluorophenyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 45%; m.p. 174° C.; ¹H NMR (500 MHz, DMSO-d₆) δ 11.03 (s, 1H), 9.22 (s, 1H), 8.08 (s, 1H), 7.62 (td, J=8.9, 6.2 Hz, 1H), 7.33 (ddd, J=10.6, 9.1, 2.9 Hz, 1H), 7.18-7.03 (m, 1H), 3.54 (s, 1H), 1.70 (s, 2H), 1.42 (s, 9H), 1.25 (s, 6H), 1.04 (s, 9H). ¹³C NMR (126 MHz, DMSO-d₆) δ 161.2, 159.4 (dd, J=243.6, 11.5 Hz), 156.2 (dd, J=248.5, 12.5 Hz), 141.5, 137.0, 133.2, 128.47 (dd, J=9.6, 3.0 Hz), 123.28 (dd, J=12.1, 3.5 Hz), 120.2, 111.42 (dd, J=21.7, 3.3 Hz), 104.6 (dd, J=26.1, 24.9 Hz), 93.5, 57.0, 56.5, 32.5, 32.2, 31.9, 30.7, 30.1. ESI-MS (m/z): 446.3 (M+H⁺).

### Example 59 2-(Tert-butyl)-N-(3,4-difluorophenyl)-3-((2,4,4-trimethylpentan-2-yl)amino)-1H-imidazo[1,2-b]pyrazole-7-carboxamide

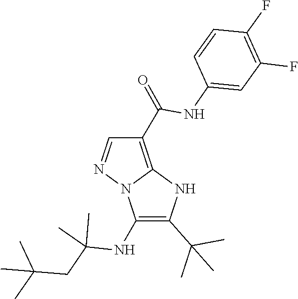 [SMILES: CC(C)(C)CC(C)(C)Nc1c(C(C)(C)C)[nH]c2c(C(=O)Nc3ccc(F)c(F)c3)cnn12]

Reaction conditions (method A): 119 mg (0.5 mmol) 5-amino-N-(3,4-difluorophenyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL MeCN, stirring at room temperature for six hours. Purification on column chromatography is necessary with Hexane:EtOAc eluent. White solid; yield: 34%; m.p. 162-164° C.; ¹H NMR (500 MHz, DMSO-d₆) δ 11.12 (s, 1H), 9.64 (s, 1H), 8.11 (s, 1H), 7.95 (ddd, J=13.8, 7.6, 2.4 Hz, 1H), 7.47-7.33 (m, 2H), 3.53 (s, 1H), 1.69 (s, 2H), 1.42 (s, 9H), 1.24 (s, 6H), 1.04 (s, 9H). ¹³C NMR (126 MHz, DMSO-d₆) δ 161.5, 149.31 (dd, J=242.0, 13.0 Hz), 145.16 (dd, J=240.2, 12.7 Hz), 140.8, 137.58 (dd, J=9.3, 2.5 Hz), 137.5, 133.3, 120.1, 117.61 (d, J=17.6 Hz), 115.93 (dd, J=5.0, 3.1 Hz), 108.68 (d, J=21.9 Hz), 94.0, 57.0, 56.5, 32.6, 32.2, 31.9, 30.7, 30.1. ESI-MS (m/z): 446.3 (M+H⁺).

### Example 60 2-(Tert-butyl)-N-(4-fluorophenyl)-3-((tert-butyl-2-yl)amino)-1H-imidazo[1,2-b]pyrazole-7-carboxamide

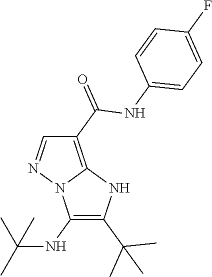 [SMILES: CC(C)(C)Nc1c(C(C)(C)C)[nH]c2c(C(=O)Nc3ccc(F)cc3)cnn12]

Reaction conditions (method A): 110 mg (0.5 mmol) 5-amino-N-(4-fluorophenyl)-1H-pyrazole-4-carboxamide I; 46 mg (1.1 equiv.) tert-butyl isocyanide III and 47 mg (1.1 equiv.) pivalaldehyde II in 0.5 mL THF, stirring at room temperature for six hours. Isolation by simple filtration and washing with cold THF. Yield: 48%. C₂₀H₂₆FN₅O₁; ¹H-NMR (500 MHz, DMSO) δ 11.02 (s, 1H), 9.45 (s, 1H), 8.08 (s, 1H), 7.70 (dd, J=7.9, 5.2 Hz, 2H), 7.12 (t, J=8.6 Hz, 2H), 3.65 (s, 1H), 1.41 (d, J=16.9 Hz, 10H), 1.19 (s, 10H). ESI-MS (m/z): 372.2 (M+H⁺).

### Example 61 2-(Tert-butyl)-N-(4-fluorophenyl)-3-((cyclohexyl-2-yl)amino)-1H-imidazo[1,2-b]pyrazole-7-carboxamide

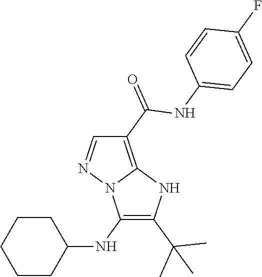 [SMILES: CC(C)(C)c1[nH]c2c(C(=O)Nc3ccc(F)cc3)cnn2c1NC1CCCCC1]

Reaction conditions (method A): 110 mg (0.5 mmol) 5-amino-N-(4-fluorophenyl)-1H-pyrazole-4-carboxamide I; 59 mg (1.1 equiv.) cyclohexyl isocyanide III and 47 mg (1.1 equiv.) pivalaldehyde II in 0.5 mL THF, stirring at room temperature for six hours. Isolation by simple filtration and washing with cold THF. Yield: 53%; C₂₂H₂₈FN₅O; ¹H-NMR (500 MHz, DMSO) δ 11.01 (s, 1H), 9.41 (s, 1H), 8.08 (s, 1H), 7.66 (dd, J=7.9, 5.2 Hz, 2H), 7.15 (t, J=8.6 Hz, 2H), 4.56 (s, 1H), 1.21-1.88 (m, 10H), 1.19 (s, 10H). ESI-MS (m/z): 398.2 v (M+H⁺).

### Example 62 2-(Tert-butyl)-3-(tert-butylamino)-N-(4-fluorophenyl)-6-methyl-1H-imidazo[1,2-b]pyrazole-7-carboxamide

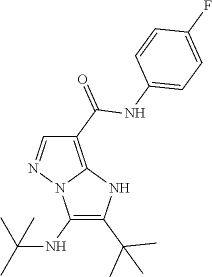 [SMILES: CC(C)(C)Nc1c(C(C)(C)C)[nH]c2c(C(=O)Nc3ccc(F)cc3)cnn12]

Reaction conditions (method A): 117 mg (0.5 mmol) 5-amino-N-(4-fluorophenyl)-3-methyl-1H-pyrazole-4-carboxamide I; 46 mg (1.1 equiv.) tert-butyl isocyanide III and 47 mg (1.1 equiv.) pivalaldehyde II in 0.5 mL THF, stirring at room temperature for six hours. Isolation by simple filtration and washing with cold THF. Yield: 39%; C₂₁H₂₈FN₅O; ¹H NMR (500 MHz, DMSO) δ 10.66 (s, 1H), 8.79 (s, 1H), 7.60 (dd, J=8.7, 5.1 Hz, 2H), 7.13 (t, J=8.8 Hz, 2H), 2.41 (s, 3H), 1.71 (s, 2H), 1.40 (s, 9H), 1.03 (s, 9H). ESI-MS (m/z): 386.2 (M+H⁺)).

### Example 63 2-(Tert-butyl)-3-(tert-butylamino)-N-(4-(trifluoromethyl)phenyl)-1H-imidazo[1,2-b]pyrazole-7-carboxamide

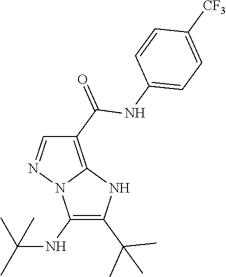 [SMILES: CC(C)(C)Nc1c(C(C)(C)C)[nH]c2c(C(=O)Nc3ccc(C(F)(F)F)cc3)cnn12]

Reaction conditions (method A): 135 mg (0.5 mmol) 5-amino-N-(4-(trifluoromethyl)phenyl)-1H-pyrazole-4-carboxamide I; 46 mg (1.1 equiv.) tert-butyl isocyanide III and 47 mg (1.1 equiv.) pivalaldehyde II in 0.5 mL THF, stirring at room temperature for six hours. Isolation by simple filtration and washing with cold THF. Yield: 49%; C₂₁H₂₆F₃N₅O; ESI-MS (m/z): 422.2 (M+H⁺).

### Example 64 2-(Tert-butyl)-3-(tert-butylamino)-N-(3-(trifluoromethyl)phenyl)-1H-imidazo[1,2-b]pyrazole-7-carboxamide

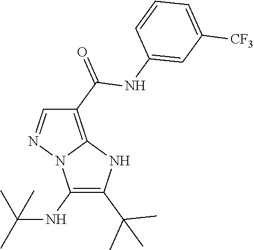 [SMILES: CC(C)(C)Nc1c(C(C)(C)C)[nH]c2c(C(=O)Nc3cccc(C(F)(F)F)c3)cnn12]

Reaction conditions (method A): 135 mg (0.5 mmol) 5-amino-N-(3-(trifluoromethyl)phenyl)-1H-pyrazole-4-carboxamide I; 46 mg (1.1 equiv.) tert-butyl isocyanide III and 47 mg (1.1 equiv.) pivalaldehyde II in 0.5 mL THF, stirring at room temperature for six hours. Isolation by simple filtration and washing with cold THF. Yield: 59%. C₂₁H₂₆F₃N₅O, ESI-MS (m/z): 422.2 (M+H⁺).

### Example 65 2-(Tert-butyl)-3-(tert-butylamino)-N-(4-chloro-3-(trifluoromethyl)phenyl)-1H-imidazo[1,2-b]pyrazole-7-carboxamide

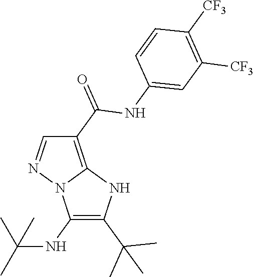 [SMILES: CC(C)(C)Nc1c(C(C)(C)C)[nH]c2c(C(=O)Nc3ccc(C(F)(F)F)c(C(F)(F)F)c3)cnn12]

Reaction conditions (method A): 152 mg (0.5 mmol) 5-amino-N-(4-chloro-3-(trifluoromethyl)phenyl)-1H-pyrazole-4-carboxamide I; 46 mg (1.1 equiv.) tert-butyl isocyanide III and 47 mg (1.1 equiv.) pivalaldehyde II in 0.5 mL THF, stirring at room temperature for six hours. Isolation by simple filtration and washing with cold MeCN. Yield: 55%. C₂₁H₂₅ClF₃N₅O, ESI-MS (m/z): 456.2 (M+H⁺).

### Example 66 2-(Tert-butyl)-3-(tert-butylamino)-N-(5-fluoropyridin-2-yl)-1H-imidazo[1,2-b]pyrazole-7-carboxamide

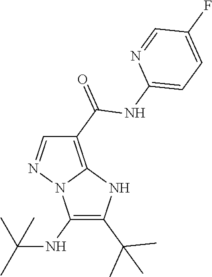 [SMILES: CC(C)(C)Nc1c(C(C)(C)C)[nH]c2c(C(=O)Nc3ccc(F)cn3)cnn12]

Reaction conditions (method A): 110 mg (0.5 mmol) 5-amino-N-(5-fluoropyridin-2-yl)-1H-pyrazole-4-carboxamide I; 46 mg (1.1 equiv.) tert-butyl isocyanide III and 47 mg (1.1 equiv.) pivalaldehyde II in 0.5 mL THF, stirring at room temperature for six hours. Isolation by simple filtration and washing with cold MeCN. Yield: 42%. C₁₉H₂₅FN₆O; ¹H NMR (500 MHz, CDCl₃) δ 8.86 (s, 1H), 8.30 (dd, J=9.1, 4.0 Hz, 1H), 8.13 (d, J=2.6 Hz, 2H), 7.85 (s, 1H), 7.44 (m, 1H), 1.44 (s, 9H), 1.30 (s, 9H). ESI-MS (m/z): 373.2 (M+H⁺).

### Example 67 Methyl 2-((7-((4-fluorophenyl)carbamoyl)-2-(4-(trifluoromethyl)phenyl)-1H-imidazo[1,2-b]pyrazol-3-yl)amino)acetate

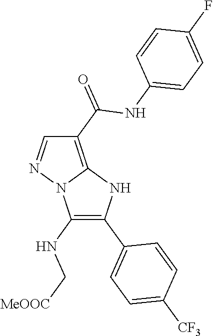 [SMILES: COC(=O)CNc1c(-c2ccc(C(F)(F)F)cc2)[nH]c2c(C(=O)Nc3ccc(F)cc3)cnn12]

Reaction conditions (method B): 110 mg (0.5 mmol) 5-amino-N-(4-fluorophenyl)-1H-pyrazole-4-carboxamide I; 54 mg (1.1 equiv.) methyl 2-isocyanoacetate III and 96 mg (1.1 equiv.) 4-trifluoromethyl benzaldehide II in 0.5 mL EtOH/0.5 mL water, stirring at room temperature for an hour. Isolation with simple filtration. Yield: 32% C₂₂H₁₇F₄N₅O₃, ESI-MS (m/z): 476.1 (M+H⁺).

### Example 68 Methyl 2-((2-(4-fluoro-3-(trifluoromethyl)phenyl)-7-((4-fluorophenyl)carbamoyl)-1H-imidazo[1,2-b]pyrazol-3-yl)amino)acetate

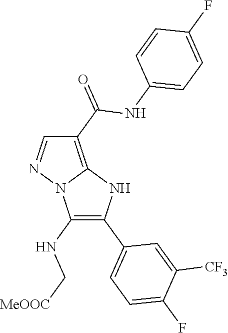 [SMILES: COC(=O)CNc1c(-c2ccc(F)c(C(F)(F)F)c2)[nH]c2c(C(=O)Nc3ccc(F)cc3)cnn12]

Reaction conditions (method B): 110 mg (0.5 mmol) 5-amino-N-(4-fluorophenyl)-1H-pyrazole-4-carboxamide I; 54 mg (1.1 equiv.) methyl 2-isocyanoacetate III and 106 mg (1.1 equiv.) 3-fluoro-4-trifluoromethyl benzaldehide II in 0.5 mL EtOH/0.5 mL water, stirring at room temperature for an hour. Isolation with simple filtration. Yield: 44%; C₂₂H₁₆F₅N₅O₃, ESI-MS (m/z): 494.1 (M+H⁺).

### Example 69 Methyl 2-((2-(2,4-bis(trifluoromethyl)phenyl)-7-((4-fluorophenyl)carbamoyl)-1H-imidazo[1,2-b]pyrazol-3-yl)amino)acetate

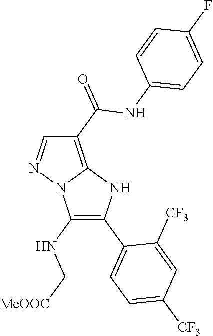 [SMILES: COC(=O)CNc1c(-c2ccc(C(F)(F)F)cc2C(F)(F)F)[nH]c2c(C(=O)Nc3ccc(F)cc3)cnn12]

Reaction conditions (method B): 110 mg (0.5 mmol) 5-amino-N-(4-fluorophenyl)-1H-pyrazole-4-carboxamide I; 54 mg (1.1 equiv.) methyl 2-isocyanoacetate III and 133 mg (1.1 equiv.) 2,4-bis-trifluoromethyl benzaldehide II in 0.5 mL EtOH/0.5 mL water, stirring at room temperature for an hour. Isolation with simple filtration. Yield: 41%; C₂₃H₁₆F₇N₅O₃; ESI-MS (m/z): 544.1 (M+H⁺).

### Example 70 Methyl 2-((2-(3,5-bis(trifluoromethyl)phenyl)-7-((4-fluorophenyl)carbamoyl)-1H-imidazo[1,2-b]pyrazol-3-yl)amino)acetate

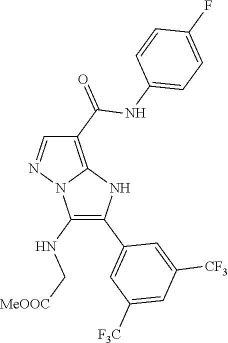 [SMILES: COC(=O)CNc1c(-c2cc(C(F)(F)F)cc(C(F)(F)F)c2)[nH]c2c(C(=O)Nc3ccc(F)cc3)cnn12]

Reaction conditions (method B): 110 mg (0.5 mmol) 5-amino-N-(4-fluorophenyl)-1H-pyrazole-4-carboxamide I; 54 mg (1.1 equiv.) methyl 2-isocyanoacetate III and 133 mg (1.1 equiv.) 3,5-bis-trifluoromethyl benzaldehide II in 0.5 mL EtOH/0.5 mL water, stirring at room temperature for an hour. Isolation with simple filtration. Yield: 39%; C₂₃H₁₆F₇N₅O₃; ESI-MS (m/z): 544.1 (M+H⁺).

### Example 71 2-(Tert-butyl)-N-(4-fluorobenzyl)-3-((2,4,4-trimethylpentan-2-yl)amino)-1H-imidazo[1,2-b]pyrazole-7-carboxamide (73)

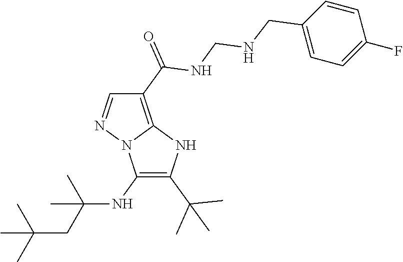 [SMILES: CC(C)(C)CC(C)(C)Nc1c(C(C)(C)C)[nH]c2c(C(=O)NCNCc3ccc(F)cc3)cnn12]

Reaction conditions (method B): 117 mg (0.5 mmol) 5-amino-N-(4-fluorobenzyl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL EtOH/0.5 mL water, stirring at room temperature for an hour. Isolation with simple filtration. White solid; yield: 33%; m.p. 163-164° C.; ¹H NMR (500 MHz, DMSO-d₆) δ 10.82 (s, 1H), 8.16 (t, J=6.1 Hz, 1H), 7.90 (s, 1H), 7.46-7.25 (m, 2H), 7.21-7.01 (m, 2H), 4.43 (d, J=6.0 Hz, 2H), 3.48 (s, 1H), 1.68 (s, 2H), 1.40 (s, 9H), 1.24 (s, 6H), 1.03 (s, 9H). ¹³C NMR (126 MHz, DMSO-d₆) δ 162.8, 161.5 (d, J=241.7 Hz), 141.0, 137.3, 136.6, 132.9, 129.5 (d, J=8.1 Hz), 120.0, 115.4 (d, J=21.2 Hz), 93.8, 56.9, 56.5, 41.5, 32.5, 32.2, 31.9, 30.7, 30.1. ESI-MS (m/z): 442.4 (M+H⁺).

### Example 72 2-(Tert-butyl)-N-(5-fluoropyridin-2-yl)-3-((2,4,4-trimethylpentan-2-yl)amino)-1H-imidazo[1,2-b]pyrazole-7-carboxamide (74)

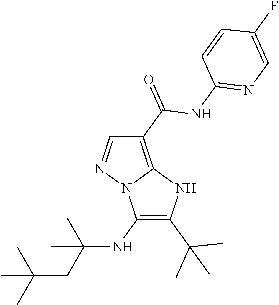 [SMILES: CC(C)(C)CC(C)(C)Nc1c(C(C)(C)C)[nH]c2c(C(=O)Nc3ccc(F)cn3)cnn12]

Reaction conditions (method B): 111 mg (0.5 mmol) 5-amino-N-(5-fluoropyridin-2-yl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL EtOH/0.5 mL water, stirring at room temperature for an hour. Isolation with simple filtration. White solid; yield: 34%; m.p. 140° C.; ¹H NMR (500 MHz, DMSO-d₆) δ 11.16 (s, 1H), 10.17 (s, 1H), 8.34 (d, J=3.1 Hz, 11H), 8.28 (s, 1H), 8.25 (dd, J=9.2, 4.2 Hz, 1H), 7.74 (td, J=8.8, 3.1 Hz, 1H), 3.53 (s, 1H), 1.69 (s, 2H), 1.43 (s, 9H), 1.24 (s, 6H), 1.04 (s, 9H). ¹³C NMR (126 MHz, DMSO-d₆) δ 161.4, 155.7 (d, J=247.1 Hz), 150.0, 141.9, 137.2, 135.3 (d, J=24.7 Hz), 133.2, 125.5 (d, J=19.4 Hz), 120.2, 115.4 (d, J=4.0 Hz), 93.8, 57.0, 56.5, 32.6, 32.2, 31.9, 30.7, 30.1. ESI-MS (m/z): 429.2 (M+H⁺).

### Example 73 2-(Tert-butyl)-N-(6-fluoropyridin-3-yl)-3-((2,4,4-trimethylpentan-2-yl)amino)-1H-imidazo[1,2-b]pyrazole-7-carboxamide (75)

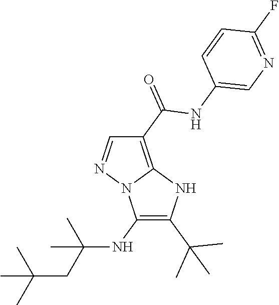 [SMILES: CC(C)(C)CC(C)(C)Nc1c(C(C)(C)C)[nH]c2c(C(=O)Nc3ccc(F)nc3)cnn12]

Reaction conditions (method B): 111 mg (0.5 mmol) 5-amino-N-(5-fluoropyridin-3-yl)-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL EtOH/0.5 mL water, stirring at room temperature for an hour. Isolation with simple filtration. White solid; yield: 25%; m.p. 177-178° C.; ¹H NMR (500 MHz, DMSO-d₆) δ 11.15 (s, 1H), 9.72 (s, 1H), 8.58 (s, 1H), 8.29-8.19 (m, 1H), 8.12 (s, 1H), 7.17 (dd, J=9.0, 3.0 Hz, 1H), 3.54 (s, 1H), 1.70 (s, 2H), 1.43 (s, 9H), 1.25 (s, 6H), 1.04 (s, 9H). ¹³C NMR (126 MHz, DMSO-d₆) δ 161.48, 158.64 (d, J=231.2 Hz), 140.91, 138.29 (d, J=15.4 Hz), 137.43, 135.37, 133.37, 120.17, 109.53 (d, J=39.4 Hz), 93.77, 56.99, 56.46, 32.57, 32.23, 31.86, 30.69, 30.10. ESI-MS (m/z): 429.2 (M+H⁺).

### Example 74 2-(Tert-butyl)-N-(4-fluorophenyl)-6-methyl-3-((2,4,4-trimethylpentan-2-yl)amino)-1H-imidazo[1,2-b]pyrazole-7-carboxamide (77)

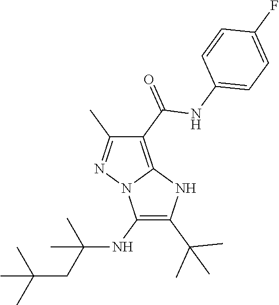 [SMILES: Cc1nn2c(NC(C)(C)CC(C)(C)C)c(C(C)(C)C)[nH]c2c1C(=O)Nc1ccc(F)cc1]

Reaction conditions (method B): 117 mg (0.5 mmol) 5-amino-N-(4-fluorophenyl)-3-methyl-1H-pyrazole-4-carboxamide I; 77 mg (1.1 equiv.) 1,1,3,3-tetramethylbutyl isocyanide III and 47 mg (1.1 equiv.) pivalaldehyde II in 0.5 mL EtOH/0.5 mL water, stirring at room temperature for an hour. Isolation with simple filtration. White solid; yield: 52%; m.p. 179° C.; 1H NMR (500 MHz, DMSO-d₆) δ 10.68 (s, 1H), 8.82 (s, 1H), 7.63 (dd, J=8.9, 5.1 Hz, 2H), 7.16 (t, J=8.7 Hz, 2H), 3.49 (s, 1H), 2.44 (s, 3H), 1.73 (s, 2H), 1.43 (s, 9H), 1.25 (s, 6H), 1.05 (s, 9H). ¹³C NMR (126 MHz, DMSO-d₆) δ 161.9, 158.4 (d, J=239.0 Hz), 151.9, 136.3, 136.3, 131.8, 123.3 (d, J=7.5 Hz), 120.0, 115.3 (d, J=22.0 Hz), 91.4, 56.9, 56.5, 32.4, 32.2, 31.9, 30.8, 30.1, 15.7. ESI-MS (m/z): 442.3 (M+H⁺).

### Example 75 Methyl 2-((7-((4-fluorophenyl)carbamoyl)-2-(4-(trifluoromethoxy)phenyl)-1H-imidazo[1,2-b]pyrazol-3-yl)amino)acetate

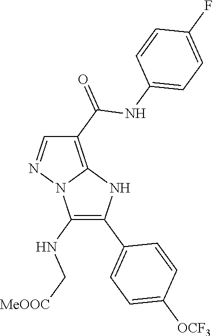 [SMILES: COC(=O)CNc1c(-c2ccc(OC(F)(F)F)cc2)[nH]c2c(C(=O)Nc3ccc(F)cc3)cnn12]

Reaction conditions (method B): 110 mg (0.5 mmol) 5-amino-N-(4-fluorophenyl)-1H-pyrazole-4-carboxamide I; 54 mg (1.1 equiv.) methyl 2-isocyanoacetate III and 105 mg (1.1 equiv.) 4-(trifluoromethoxy)benzaldehyde II in 0.5 mL EtOH/0.5 mL water, stirring at room temperature for an hour. Isolation with simple filtration. Yield: 31%; C₂₂H₁₇F₄N₅O₄; ESI-MS (m/z): 492.1 (M+H⁺).

### Example 76 3-(Tert-butylamino)-2-cyclopropyl-N-(4-fluorophenyl)-1H-imidazo[1,2-b]pyrazole-7-carbox-amide

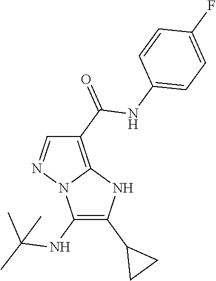 [SMILES: CC(C)(C)Nc1c(C2CC2)[nH]c2c(C(=O)Nc3ccc(F)cc3)cnn12]

Reaction conditions (method B): 110 mg (0.5 mmol) 5-amino-N-(4-fluorophenyl)-1H-pyrazole-4-carboxamide I; 46 mg (1.1 equiv.) tert-butyl isocyanide III and 39 mg (1.1 equiv.) cyclopropyl aldehyde II in 0.5 mL EtOH/0.5 mL water, stirring at room temperature for an hour. Isolation with simple filtration. Yield: 28%; C₁₉H₂₂FN₅O; ESI-MS (m/z): 356.1 (M+H⁺).

### Example 77 N-(4-bromophenyl)-2-(tert-butyl)-3-(tert-butylamino)-1H-imidazo[1,2-b]pyrazole-7-carbox-amide

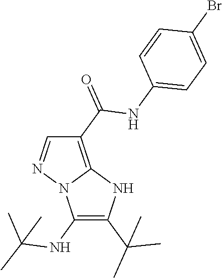 [SMILES: CC(C)(C)Nc1c(C(C)(C)C)[nH]c2c(C(=O)Nc3ccc(Br)cc3)cnn12]

Reaction conditions (method B): 140 mg (0.5 mmol) 5-amino-N-(4-bromophenyl)-1H-pyrazole-4-carboxamide I; 46 mg (1.1 equiv.) tert-butyl isocyanide III and 47 mg (1.1 equiv.) pivalaldehyde U1 in 0.5 mL EtOH/0.5 mL water, stirring at room temperature for an hour. Isolation with simple filtration. Yield: 32%; C₂₀H₂₆BrN₅O; ESI-MS (m/z): 432.1 (M+H⁺).

### Example 78 2-(Tert-butyl)-3-(tert-butylamino)-N-(4-nitrophenyl)-1H-imidazo[1,2-b]pyrazole-7-carbox-amide

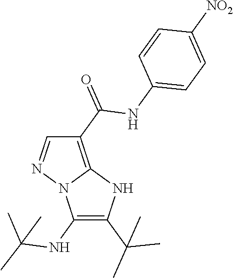 [SMILES: CC(C)(C)Nc1c(C(C)(C)C)[nH]c2c(C(=O)Nc3ccc([N+](=O)[O-])cc3)cnn12]

Reaction conditions (method B): 124 mg (0.5 mmol) 5-amino-N-(4-nitrophenyl)-1H-pyrazole-4-carboxamide I; 46 mg (1.1 equiv.) tert-butyl isocyanide III and 47 mg (1.1 equiv.) pivalaldehyde II in 0.5 mL EtOH/0.5 mL water, stirring at room temperature for an hour. Isolation with simple filtration. Yield: 38%; C₂₀H₂₆N₆O₃; ¹H NMR (500 MHz, DMSO) δ 11.23 (s, 1H), 10.03 (s, 1H), 8.22 (d, J=9.6 Hz, 2H), 8.16 (s, 1H), 7.97 (d, J=9.6 Hz, 2H), 1.42 (s, 9H), 1.20 (s, 9H). ESI-MS (m/z): 399.2 (M+H).

### Example 79 3-(Tert-butylamino)-2-cyclopropyl-N-(4-nitrophenyl)-1H-imidazo[1,2-b]pyrazole-7-carboxamide

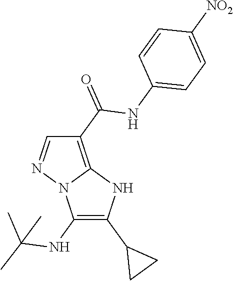 [SMILES: CC(C)(C)Nc1c(C2CC2)[nH]c2c(C(=O)Nc3ccc([N+](=O)[O-])cc3)cnn12]

Reaction conditions (method B): 124 mg (0.5 mmol) 5-amino-N-(4-nitrophenyl)-1H-pyrazole-4-carboxamide I; 46 mg (1.1 equiv.) tert-butyl isocyanide III and 39 mg (1.1 equiv.) cyclopropyl aldehyde II in 0.5 mL EtOH/0.5 mL water, stirring at room temperature for an hour. Isolation with simple filtration. Yield: 30%; C₁₉H₂₂N₆O₃; ESI-MS (m/z): 383.2 (M+H⁺).

### Example 80 2-(Tert-butyl)-3-(tert-butylamino)-N-(2-methyl-4-nitrophenyl)-1H-imidazo[1,2-b]pyrazole-7-carboxamide

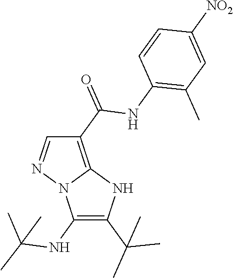 [SMILES: Cc1cc([N+](=O)[O-])ccc1NC(=O)c1cnn2c(NC(C)(C)C)c(C(C)(C)C)[nH]c12]

Reaction conditions (method B): 131 mg (0.5 mmol) 5-amino-N-(2-methyl-4-nitrophenyl)-1H-pyrazole-4-carboxamide I; 46 mg (1.1 equiv.) tert-butyl isocyanide III and 47 mg (1.1 equiv.) pivalaldehyde II in 0.5 mL EtOH/0.5 mL water, stirring at room temperature for an hour. Isolation with simple filtration. Yield: 36%; C₂₁H₂₈N₆O₃; ESI-MS (m/z): 413.2 (M+H⁺).

### Example 81 2-(Tert-butyl)-3-(tert-butylamino)-N-(3-hydroxy-4-nitrophenyl)-1H-imidazo[1,2-b]pyrazole-7-carboxamide

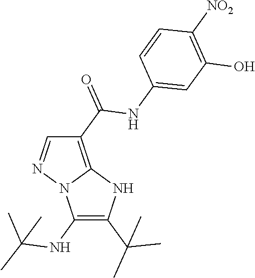 [SMILES: CC(C)(C)Nc1c(C(C)(C)C)[nH]c2c(C(=O)Nc3ccc([N+](=O)[O-])c(O)c3)cnn12]

Reaction conditions (method B): 131 mg (0.5 mmol) 5-amino-N-(3-hydroxy-4-nitrophenyl)-1H-pyrazole-4-carboxamide I; 46 mg (1.1 equiv.) tert-butyl isocyanide III and 47 mg (1.1 equiv.) pivalaldehyde II in 0.5 mL EtOH/0.5 mL water, stirring at room temperature for an hour. Purification on column chromatography was required. Yield: 36%; C₂₀H₂₆N₆O₄; ESI-MS (m/z): 415.2 (M+H⁺).

### Example 82 2-(Tert-butyl)-N-(3-hydroxy-4-nitrophenyl)-3-((2,4,4-trimethylpentan-2-yl)amino)-1H-imidazo[1,2-b]pyrazole-7-carboxamide

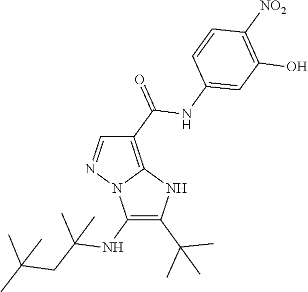 [SMILES: CC(C)(C)CC(C)(C)Nc1c(C(C)(C)C)[nH]c2c(C(=O)Nc3ccc([N+](=O)[O-])c(O)c3)cnn12]

Reaction conditions (method B): 131 mg (0.5 mmol) 5-amino-N-(3-hydroxy-4-nitrophenyl)-1H-pyrazole-4-carboxamide I; 76 mg (1.1 equiv.) Walborsky-reagent III and 47 mg (1.1 equiv.) pivalaldehyde II in 0.5 mL EtOH/0.5 mL water, stirring at room temperature for an hour. Purification on column chromatography was required. Yield: 31%; C₂₄H₃₄N₆O₄; ¹H NMR (500 MHz, DMSO) δ 11.27 (s, 1H), 11.13 (s, 1H), 9.02 (s, 1H), 8.16 (s, 1H), 7.77 (dd, J=8.9, 2.6 Hz, 11H), 7.71 (d, J=2.6 Hz, 1H), 3.57 (s, 1H), 1.70 (s, 2H), 1.50 (s, 2H), 1.42 (s, 9H), 1.24 (s, 5H), 1.04 (s, 8H). ESI-MS (m/z): 471.3 (M+H⁺).

### Example 83 N-(4-Aminophenyl)-2-(tert-butyl)-3-(tert-butylamino)-1H-imidazo[1,2-b]pyrazole-7-carboxamide

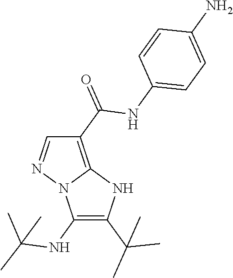 [SMILES: CC(C)(C)Nc1c(C(C)(C)C)[nH]c2c(C(=O)Nc3ccc(N)cc3)cnn12]

Reaction conditions (method B): 109 mg (0.5 mmol) 5-amino-N-(4-aminophenyl)-1H-pyrazole-4-carboxamide I; 46 mg (1.1 equiv.) tert-butyl isocyanide III and 47 mg (1.1 equiv.) pivalaldehyde II in 0.5 mL EtOH/0.5 mL water, stirring at room temperature for half an hour. Isolated by simple filtration. Yield: 48% (method A: 56%, flash chromatography); C₂₀H₂₈N₆O; ¹H NMR (500 MHz, D6MSO) δ 10.89 (s, 1H), 9.00 (s, 1H), 8.12 (s, 1H), 8.00 (s, 1H), 7.25 (d, J=8.6 Hz, 2H)), 6.55 (d, J=8.6 Hz, 2H), 1.41 (s, 9H), 1.20 (s, 9H). 13C-NMR (125 MHz, D6MSO) δ 160.6, 144.4, 140.5, 136.6, 132.8, 128.9, 122.2, 120.0, 114.1, 94.3, 52.4, 30.8 and 30.1. ESI-MS (m/z): 369.2 (M+H⁺).

### Example 84 2-(Tert-butyl)-3-(tert-butylamino)-N-(4-(dimethylamino)phenyl)-1H-imidazo[1,2-b]pyrazole-7-carboxamide

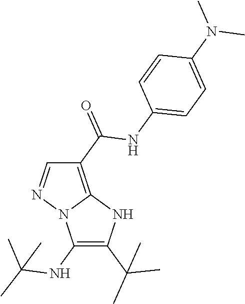 [SMILES: CN(C)c1ccc(NC(=O)c2cnn3c(NC(C)(C)C)c(C(C)(C)C)[nH]c23)cc1]

Reaction conditions (method B): 122 mg (0.5 mmol) 5-amino-N-(4-(dimethylamino)phenyl)-1H-pyrazole-4-carboxamide I; 46 mg (1.1 equiv.) tert-butyl isocyanide III and 47 mg (1.1 equiv.) pivalaldehyde II in 0.5 mL EtOH/0.5 mL water, stirring at room temperature for half an hour. Isolated by simple filtration. Yield: 53%; C₂₂H₃₂N₆O; ¹H NMR (500 MHz, DMSO) δ 10.93 (s, 1H), 9.13 (s, 1H), 8.04 (s, 1H), 7.49 (s, 2H), 6.70 (s, 2H), 2.84 (s, 6H), 1.40 (s, 9H), 1.20 (s, 9H). ESI-MS (m/z): 397.3 (M+H⁺).

### Example 85 N-(4-aminophenyl)-3-(tert-butylamino)-2-cyclopropyl-1H-imidazo[1,2-b]pyrazole-7-carbox-amide

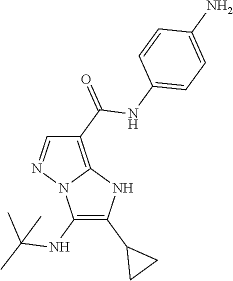 [SMILES: CC(C)(C)Nc1c(C2CC2)[nH]c2c(C(=O)Nc3ccc(N)cc3)cnn12]

Reaction conditions (method B): 109 mg (0.5 mmol) 5-amino-N-(4-aminophenyl)-1H-pyrazole-4-carboxamide I; 46 mg (1.1 equiv.) tert-butyl isocyanide III and 36 mg (1.1 equiv.) cyclopropyl aldehyde II in 0.5 mL EtOH/0.5 mL water, stirring at room temperature for an hour. Isolation after flash chromatography. Yield: 27%; C₁₉H₂₄N₆O; ESI-MS (m/z): 353.3 (M+H⁺).

### Example 86 N-(4-Amino-3-hydroxyphenyl)-2-(tert-butyl)-3-(tert-butylamino)-1H-imidazo[1,2-b]pyrazole-7-carboxamide

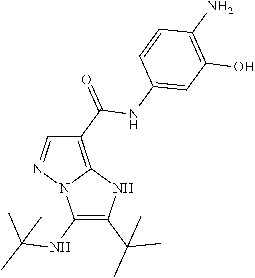 [SMILES: CC(C)(C)Nc1c(C(C)(C)C)[nH]c2c(C(=O)Nc3ccc(N)c(O)c3)cnn12]

Reaction conditions (method B): 117 mg (0.5 mmol) 5-amino-N-(4-amino-3-hydroxyphenyl)-1H-pyrazole-4-carboxamide I; 46 mg (1.1 equiv.) tert-butyl isocyanide III and 47 mg (1.1 equiv.) pivalaldehyde II in 0.5 mL EtOH/0.5 mL water, stirring at room temperature for half an hour. Isolated by simple filtration. Yield: 44%; C₂₀H₂₈N₆O₂; ¹H NMR (500 MHz, DMSO) δ 10.98 (s, 1H), 9.66 (s, 1H), 9.02 (s, 1H), 8.04 (s, 1H), 6.90 (d, J=8.4 Hz, 1H), 6.14 (s, 1H), 6.05 (dd, 1H), 4.86 (s, 2H), 3.68 (s, 1H), 1.39 (s, 9H), 1.21 (s, 9H). ESI-MS (m/z): 385.2 (M+H⁺).

### Example 87 N-(4-amino-2-methylphenyl)-2-(tert-butyl)-3-(tert-butylamino)-1H-imidazo[1,2-b]pyrazole-7-carboxamide

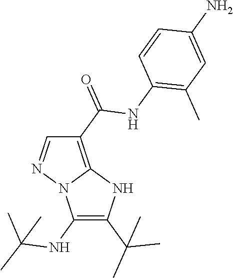 [SMILES: Cc1cc(N)ccc1NC(=O)c1cnn2c(NC(C)(C)C)c(C(C)(C)C)[nH]c12]

Reaction conditions (method B): 117 mg (0.5 mmol) 5-amino-N-(4-amino-2-methylphenyl)-1H-pyrazole-4-carboxamide I; 46 mg (1.1 equiv.) tert-butyl isocyanide III and 47 mg (1.1 equiv.) pivalaldehyde II in 0.5 mL EtOH/0.5 mL water, stirring at room temperature for half an hour. Isolated by simple filtration. Yield: 39%; C₂₁H₃₀N₆O; ESI-MS (m/z): 383.2 (M+H⁺).

### Example 88 2-(Tert-butyl)-3-(tert-butylamino)-N-(4-(methylthio)phenyl)-1H-imidazo[1,2-b]pyrazole-7-carboxamide

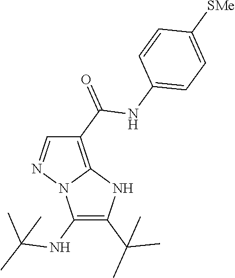 [SMILES: CSc1ccc(NC(=O)c2cnn3c(NC(C)(C)C)c(C(C)(C)C)[nH]c23)cc1]

Reaction conditions (method B): 124 mg (0.5 mmol) 5-amino-N-(4-(methylthio)phenyl)-1H-pyrazole-4-carboxamide I; 46 mg (1.1 equiv.) tert-butyl isocyanide III and 47 mg (1.1 equiv.) pivalaldehyde II in 0.5 mL EtOH/0.5 mL water, stirring at room temperature for half an hour. Isolated by simple filtration. Yield: 45%; C₂₁H₂₉N₅OS; ESI-MS (m/z): 400.2 (M+H⁺).

### Example 89 N-(4-aminophenyl)-2-(tert-butyl)-3-((2,4,4-trimethylpentan-2-yl)amino)-1H-imidazo[1,2-b]pyrazole-7-carboxamide

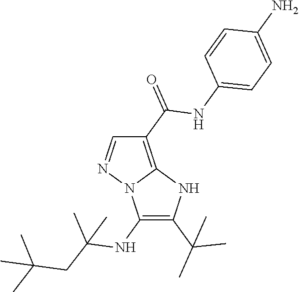 [SMILES: CC(C)(C)CC(C)(C)Nc1c(C(C)(C)C)[nH]c2c(C(=O)Nc3ccc(N)cc3)cnn12]

Reaction conditions (method B): 109 mg (0.5 mmol) 5-amino-N-(4-aminophenyl)-1H-pyrazole-4-carboxamide I; 76 mg (1.1 equiv.) Walborsky isocyanide III and 47 mg (1.1 equiv.) pivalaldehyde II in 0.5 mL EtOH/0.5 mL water, stirring at room temperature for an hour. Isolation by simple filtration. Yield: 53%; C₂₄H₃₆N₆O; ESI-MS (m/z): 425.2 (M+H⁺).

### Example 90 2-(Tert-butyl)-3-(tert-butylamino)-N-(4-hydroxyphenyl)-1H-imidazo[1,2-b]pyrazole-7-carboxamide

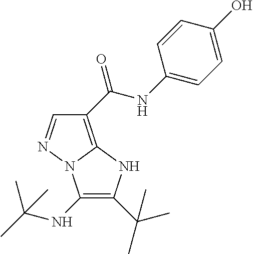 [SMILES: CC(C)(C)Nc1c(C(C)(C)C)[nH]c2c(C(=O)Nc3ccc(O)cc3)cnn12]

Reaction conditions (method B): 109 mg (0.5 mmol) 5-amino-N-(4-hydroxyphenyl)-1H-pyrazole-4-carboxamide I; 46 mg (1.1 equiv.) tert-butyl isocyanide III and 47 mg (1.1 equiv.) pivalaldehyde II in 0.5 mL EtOH/0.5 mL water, stirring at room temperature for half an hour. Isolated by simple filtration. Yield: 45%; C₂₀H₂₇N₅O₂; ESI-MS (m/z): 370.2 (M+H⁺).

### Example 91 2-(Tert-butyl)-N-(4-hydroxyphenyl)-3-((2,4,4-trimethylpentan-2-yl)amino)-1H-imidazo[1,2-b]pyrazole-7-carboxamide

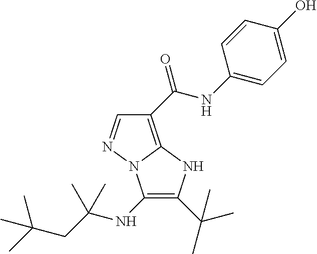 [SMILES: CC(C)(C)CC(C)(C)Nc1c(C(C)(C)C)[nH]c2c(C(=O)Nc3ccc(O)cc3)cnn12]

Reaction conditions (method B): 109 mg (0.5 mmol) 5-amino-N-(4-hydroxyphenyl)-1H-pyrazole-4-carboxamide I; 76 mg (1.1 equiv.) 1,1,3,3-tetrabutyl methyl isocyanide (Walborsky) III and 47 mg (1.1 equiv.) pivalaldehyde II in 0.5 mL EtOH/0.5 mL water, stirring at room temperature for half an hour. Isolated by simple filtration. Yield: 48%; ¹H NMR (500 MHz, DMSO) δ 10.97 (s, 1H), 9.18 (s, 1H), 9.12 (s, 1H), 8.06 (s, 1H), 7.45 (d, J=7.8 Hz, 2H), 6.71 (d, J=7.7 Hz, 2H), 1.70 (s, 2H), 1.42 (s, 9H), 1.22 (s, 6H), 1.01 (s, 9H). 13C-NMR (125 MHz, D6MSO) δ 161.1, 153.4, 140.7, 137.4, 133.0, 131.7, 122.6, 120.3, 115.9, 94.8, 57.1, 56.5, 32.3, 30.7 and 30.1. ESI-MS (m/z): 426.2 (M+H⁺).

### Example 92 2-(Tert-butyl)-3-(cyclohexylamino)-N-(4-hydroxyphenyl)-1H-imidazo[1,2-b]pyrazole-7-carbox-amide

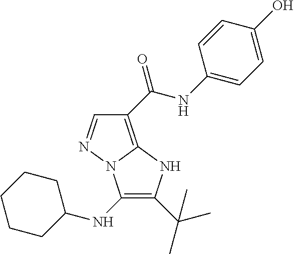 [SMILES: CC(C)(C)c1[nH]c2c(C(=O)Nc3ccc(O)cc3)cnn2c1NC1CCCCC1]

Reaction conditions (method B): 109 mg (0.5 mmol) 5-amino-N-(4-hydroxyphenyl)-1H-pyrazole-4-carboxamide I; 59 mg (1.1 equiv.) cyclohexyl isocyanide III and 47 mg (1.1 equiv.) pivalaldehyde II in 0.5 mL EtOH/0.5 mL water, stirring at room temperature for half an hour. Isolated by simple filtration. Yield: 43%; C₂₂H₂₉N₅O₂; ESI-MS (m/z): 396.2 (M+H⁺).

### Example 93 3-(cyclohexylamino)-N-(4-hydroxyphenyl)-2-(4-(trifluoromethyl)phenyl)-1H-imidazo[1,2-b]pyrazole-7-carboxamide

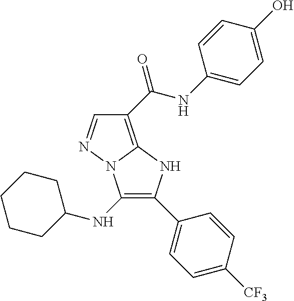 [SMILES: O=C(Nc1ccc(O)cc1)c1cnn2c(NC3CCCCC3)c(-c3ccc(C(F)(F)F)cc3)[nH]c12]

Reaction conditions (method B): 109 mg (0.5 mmol) 5-amino-N-(4-hydroxyphenyl)-1H-pyrazole-4-carboxamide I; 59 mg (1.1 equiv.) cyclohexyl isocyanide III and 96 mg (1.1 equiv.) 4-trifluoromethyl benzaldehyde II in 0.5 mL EtOH/0.5 mL water, stirring at room temperature for half an hour. Isolated by simple filtration. Yield: 33%; C₂₅H₂₄F₃N₅O₂; ESI-MS (m/z): 484.2 (M+H⁺).

### Example 94 2-(Tert-butyl)-N-(2-hydroxyphenyl)-3-((2,4,4-trimethylpentan-2-yl)amino)-1H-imidazo[1,2-b]pyrazole-7-carboxamide

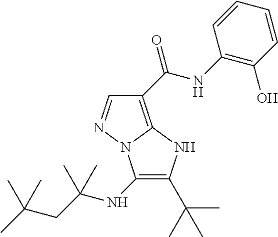 [SMILES: CC(C)(C)CC(C)(C)Nc1c(C(C)(C)C)[nH]c2c(C(=O)Nc3ccccc3O)cnn12]

Reaction conditions (method B): 109 mg (0.5 mmol) 5-amino-N-(2-hydroxyphenyl)-1H-pyrazole-4-carboxamide I; 76 mg (1.1 equiv.) 1,1,3,3-tetrabutyl methyl isocyanide (Walborsky) III and 47 mg (1.1 equiv.) pivalaldehyde II in 0.5 mL EtOH/0.5 mL water, stirring at room temperature for half an hour. Isolated by simple filtration. Yield: 49%; ¹H NMR (500 MHz, DMSO) δ 11.12 (s, 1H), 10.04 (s, 1H), 9.18 (s, 1H), 8.13 (s, 1H), 7.49 (d, J=7.8 Hz, 1H), 6.92 (ddd, J=40.1, 15.2, 7.7 Hz, 3H), 3.55 (s, 1H), 1.70 (s, 2H), 1.43 (s, 10H), 1.25 (s, 6H), 1.04 (s, 10H). ESI-MS (m/z): 426.2 (M+H⁺).

### Example 95 2-(tert-butyl)-N-(3-hydroxyphenyl)-3-((2,4,4-trimethylpentan-2-yl)amino)-1H-imidazo[1,2-b]pyrazole-7-carboxamide

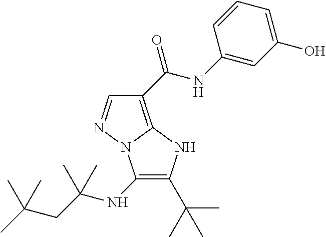 [SMILES: CC(C)(C)CC(C)(C)Nc1c(C(C)(C)C)[nH]c2c(C(=O)Nc3cccc(O)c3)cnn12]

Reaction conditions (method B): 109 mg (0.5 mmol) 5-amino-N-(3-hydroxyphenyl)-1H-pyrazole-4-carboxamide I; 76 mg (1.1 equiv.) 1,1,3,3-tetrabutyl methyl isocyanide (Walborsky) III and 47 mg (1.1 equiv.) pivalaldehyde II in 0.5 mL EtOH/0.5 mL water, stirring at room temperature for half an hour. Isolated by simple filtration. Yield: 51%; C₂₄H₃₅N₅O₂; ¹H NMR (500 MHz, DMSO) δ 11.03 (s, 1H), 9.34-9.25 (m, 2H), 8.12 (s, 1H), 7.04-7.26 (m, 3H), 6.43 (dd, J=7.9, 1.4 Hz, 1H), 3.51 (s, 1H), 1.70 (s, 2H), 1.44 (s, 9H), 1.25 (s, 6H), 1.04 (s, 9H). ESI-MS (m/z): 426.2 (M+H⁺).

### Example 96 2-(Tert-butyl)-N-(4-hydroxy-2-methylphenyl)-3-((2,4,4-trimethylpentan-2-yl)amino)-1H-imidazo[1,2-b]pyrazole-7-carboxamide

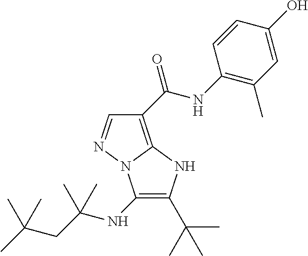 [SMILES: Cc1cc(O)ccc1NC(=O)c1cnn2c(NC(C)(C)CC(C)(C)C)c(C(C)(C)C)[nH]c12]

Reaction conditions (method B): 116 mg (0.5 mmol) 5-amino-N-(4-hydroxy-2-methylphenyl)-1H-pyrazole-4-carboxamide I; 76 mg (1.1 equiv.) 1,1,3,3-tetrabutyl methyl isocyanide (Walborsky) III and 47 mg (1.1 equiv.) pivalaldehyde II in 0.5 mL EtOH/0.5 mL water, stirring at room temperature for half an hour. Isolated by simple filtration. C₂₅H₃₇N₅O₂, ¹H NMR (500 MHz, DMSO) δ 10.87 (s, 1H), 9.22 (s, 1H), 8.75 (s, 1H), 7.97 (s, 1H), 7.03 (d, J=8.4 Hz, 1H), 6.65 (d, J=2.3 Hz, 1H), 6.59 (dd, J=8.4, 2.5 Hz, 1H), 3.51 (s, 1H), 2.13 (s, 3H), 1.70 (s, 2H), 1.43 (s, 9H), 1.25 (s, 6H), 1.04 (s, 9H). ESI-MS (m/z): 440.4 (M+H⁺).

### Example 97 2-(Tert-butyl)-3-(tert-butylamino)-N-(4-fluorophenyl)-1H-imidazo[1,2-b]pyrazole-7-carbothioamide

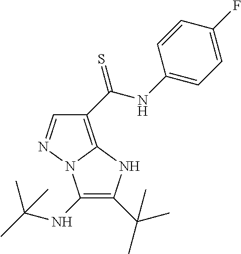 [SMILES: CC(C)(C)Nc1c(C(C)(C)C)[nH]c2c(C(=S)Nc3ccc(F)cc3)cnn12]

Reaction conditions (method B): 118 mg (0.5 mmol) 5-amino-N-(4-fluorophenyl)-1H-pyrazole-4-carbothioamide I′; 46 mg (1.1 equiv.) tert-butyl isocyanide III and 47 mg (1.1 equiv.) pivalaldehyde II in 0.5 mL EtOH/0.5 mL water, stirring at room temperature for half an hour. Isolated by simple filtration. C₂₀H₂₆FN₅S; yield: 17% yellow cristal. ¹H NMR (500 MHz, DMSO) δ 10.53 (s, 1H), 10.22 (d, J=36.2 Hz, 1H), 8.14 (s, 1H), 7.70-7.56 (m, 2H), 7.22 (t, J=8.6 Hz, 2H), 3.81 (s, 1H), 1.41 (s, 11H), 1.21 (s, 12H). ESI-MS (m/z): 388.5 (M+H⁺).

### Example 98 Ethyl 4-(2-(tert-butyl)-3-(tert-butylamino)-1H-imidazo[1,2-b]pyrazole-7-carboxamido)benzoate

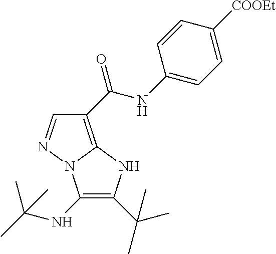 [SMILES: CCOC(=O)c1ccc(NC(=O)c2cnn3c(NC(C)(C)C)c(C(C)(C)C)[nH]c23)cc1]

Reaction conditions (method B): 137 mg (0.5 mmol) ethyl 4-(5-amino-1H-pyrazole-4-carboxamido)benzoate I; 46 mg (1.1 equiv.) tert-butyl isocyanide III and 47 mg (1.1 equiv.) pivalaldehyde II in 0.5 mL EtOH/0.5 mL water, stirring at room temperature for an hour. Flash column chromatography. Yield: 35%; C₂₃H₃₁N₅O₃; ¹H NMR (500 MHz, DMSO) δ 11.14 (s, 1H), 9.75 (s, 1H), 8.15 (s, 1H), 7.89 (s, 4H), 4.27 (d, J=5.1 Hz, 2H), 1.40 (s, 9H), 1.30 (s, 3H), 1.20 (s, 9H). ESI-MS (m/z): 426.2 (M+H⁺).

### Example 99 2-(Tert-butyl)-3-(tert-butylamino)-N-(4-(5-((3aS,4S,6aR)-2-oxohexahydro-1H-thieno[3,4-d]imidazol-4-yl)pentanamido)phenyl)-1H-imidazo[1,2-b]pyrazole-7-carboxamide

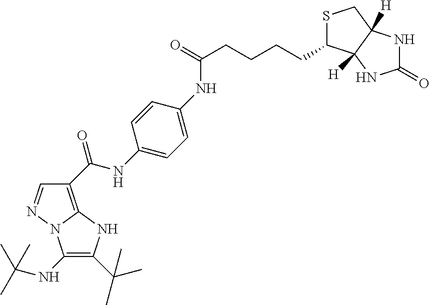 [SMILES: CC(C)(C)Nc1c(C(C)(C)C)[nH]c2c(C(=O)Nc3ccc(NC(=O)CCCC[C@@H]4SC[C@@H]5NC(=O)N[C@@H]54)cc3)cnn12]

C₃₀H₄₂N₈O₃S; ¹H NMR (500 MHz, DMSO) δ 11.01 (s, 1H), 9.75 (s, 1H), 9.33 (s, 1H), 8.08 (d, J=14.7 Hz, 1H), 7.58 (d, J=9.5 Hz, 2H), 7.50-7.42 (m, 2H), 6.41 (s, 1H), 6.33 (s, 1H), 4.28 (d, J=5.3 Hz, 2H), 4.13 (s, 2H), 3.11 (s, 2H), 2.88-2.74 (m, 2H), 2.27 (t, J=6.5 Hz, 3H), 1.58 (dd, J=29.8, 23.7 Hz, 6H), 1.41 (s, 9H), 1.19 (s, 9H). ESI-MS (m/z): 595.3 (M+H⁺).

### Example 100 2-(tert-butyl)-3-(tert-butylamino)-N-(3-(trifluoromethyl)phenyl)-1H-imidazo[1,2-b]pyrazole-7-carboxamide

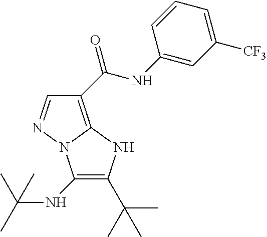 [SMILES: CC(C)(C)Nc1c(C(C)(C)C)[nH]c2c(C(=O)Nc3cccc(C(F)(F)F)c3)cnn12]

Reaction conditions (method B): 135 mg (0.5 mmol) 5-amino-N-(3-(trifluoromethyl)phenyl)-1H-pyrazole-4-carboxamide I; 46 mg (1.1 equiv.) tert-butyl isocyanide III and 47 mg (1.1 equiv.) pivalaldehyde II in 0.5 mL EtOH/0.5 mL water, stirring at room temperature for an hour. Flash column chromatography. Yield: 35%; C₂₁H₂₆F₃N₅O, ¹H NMR (500 MHz, DMSO) δ 11.15 (s, 1H), 9.75 (s, 1H), 8.34 (s, 1H), 8.16 (s, 1H), 7.91 (d, J=8.1 Hz, 11H), 7.55 (t, J=8.0 Hz, 1H), 7.37 (dd, J=21.3, 7.9 Hz, 1H), 3.54 (s, 1H), 1.70 (s, 2H), 1.44 (s, 9H), 1.05 (s, 9H). 13C-NMR (125 MHz, D6MSO) δ 161.7, 141.3, 140.9, 137.7, 133.4, 130.0, 123.2, 120.4, 118.9, 115.9, 94.3, 57.0, 56.7, 32.7, 32.2, 32.1, 30.7 and 30.1. ESI-MS (m/z): 422.2 (M+H⁺).

### Example 101 2-(tert-butyl)-N-(4-hydroxyphenyl)-3-(pentylamino)-1H-imidazo[1,2-b]pyrazole-7-carboxamide

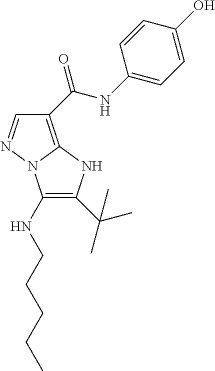 [SMILES: CCCCCNc1c(C(C)(C)C)[nH]c2c(C(=O)Nc3ccc(O)cc3)cnn12]

Reaction conditions (method B): 109 mg (0.5 mmol) 5-amino-N-(4-hydroxyphenyl)-1H-pyrazole-4-carboxamide I; 53 mg (1.1 equiv.) 1-pentyl isocyanide III and 47 mg (1.1 equiv.) pivalaldehyde II in 0.5 mL EtOH/0.5 mL water, stirring at room temperature for an hour. Purification on column chromatography. Yield: 24%; C₂₁H₂₉N₅O₂; ESI-MS (m/z): 384.2 (M+H⁺).

### BIOLOGICAL EXAMPLES

### Example 102

### In Vitro Citotoxic Effects of Various Compounds Having the General Formula (I.)

In our experiments three different cell lines were used. Cells were purchased from the American Type Culture Collection (ATCC, Manassas, Va., USA). The human breast adenocarcinoma cell line, MCF-7 cells were maintained in Dulbecco's Modified Eagle Medium/Nutrient Mixture F-12 (DMEM/F12) 10% fetal calf serum (FCS, Gibco) and mouse mammary carcinoma 4T1 and human promyelocytic leukemia, HL-60 cells were maintained in Roswell Park Memorial Institute 1640 medium (RPMI-1640) 10% FCS. Media were supplemented with 2 mM GlutaMAX, and 100 U/mL penicillin, 100 μg/mL streptomycin (Life Technologies, Carlsbad, Calif., USA). Cell cultures were maintained at 37° C. in a humidified incubator in an atmosphere of 5% CO₂ (Sanyo, Japan).

For viability assays cells were seeded in 96 well plates (MCF7 and 4T1: 6000, HL60: 120.000 cells/well) and incubated overnight. Compounds were dissolved in dimethyl sulfoxide (DMSO). Cells were treated with an increasing concentration of compounds having the general formula (I.) (156 nM-100 μM). Cell viability was determined after 72h incubation. Resazurin reagent (Sigma-Aldrich) was dissolved in PBS (pH 7.4) at 0.15 mg/ml concentration, sterile filtered (0.22 μm, Merck Millipore) and aliquoted at −20° C. Resazurin reagent (Sigma-Aldrich) was added at a final concentration of 25 μg/ml. After 2 hours incubation at 37° C. 5% CO₂ fluorescence (530 nm excitation/580 nm emission) was recorded on a multimode microplate reader (Cytofluor4000, PerSeptive Biosytems). Viability was calculated with relation to untreated control cells and blank wells containing media without cells. IC₅₀ values (50% inhibiting concentration) were calculated by GraphPad Prism® 5 (La Jolla, Calif., USA).

The determined IC₅₀ values are listed in Table 1.

It is apparent that the applied compounds exhibited a significant citotoxic effect. HL60 cells were highly susceptible to cell death following treatment with selected compounds.

TABLE 1
in vitro citotoxic activity of imidazo-pyrazole carboxamide derivatives

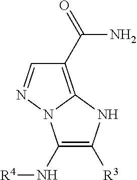

| Example | R³ | R⁴ | 4T1^{a)} | MCF-7^{a)} | HL-60^{a)} |
|---|---|---|---|---|---|
| 6 | 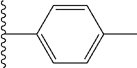 [SMILES: *c1ccc(C)cc1] | 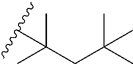 [SMILES: *C(C)(C)CC(C)(C)C] | 12.3 | 17.0 | 12.6 |
| 7 | 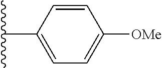 [SMILES: *c1ccc(OC)cc1] | 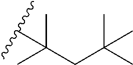 [SMILES: *C(C)(C)CC(C)(C)C] | 25.2 | 18.6 | 19.0 |
| 8 | 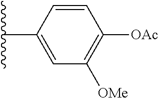 [SMILES: *c1ccc(OC(C)=O)c(OC)c1] | 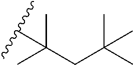 [SMILES: *C(C)(C)CC(C)(C)C] | 34.1 | 40.6 | 26.6 |
| 10 | 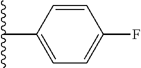 [SMILES: *c1ccc(F)cc1] | 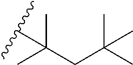 [SMILES: *C(C)(C)CC(C)(C)C] | >50 | >50 | 17.5 |
| 11 | 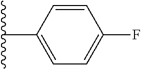 [SMILES: *c1ccc(F)cc1] | 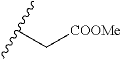 [SMILES: *CC(=O)OC] | >50 | >50 | 15.9 |
| 12 | 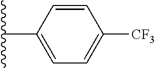 [SMILES: *c1ccc(C(F)(F)F)cc1] | 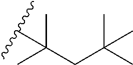 [SMILES: *C(C)(C)CC(C)(C)C] | 20.9 | 12.7 | 13.2 |
| 15 | 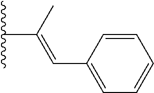 [SMILES: */C(C)=C/c1ccccc1] | 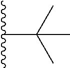 [SMILES: *C(C)(C)C] | >50 | >50 | 29.2 |
| 16 | 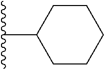 [SMILES: *C1CCCCC1] | 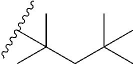 [SMILES: *C(C)(C)CC(C)(C)C] | 12.3 | 11.1 | 9.35 |
| 18 | 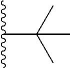 [SMILES: *C(C)(C)C] | 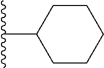 [SMILES: *C1CCCCC1] | 4.24 | 4.07 | 2.95 |
| 19 | 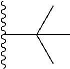 [SMILES: *C(C)(C)C] | 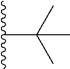 [SMILES: *C(C)(C)C] | 23.9 | 27.9 | 4.99 |
| 20 | 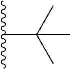 [SMILES: *C(C)(C)C] | 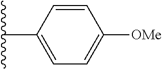 [SMILES: *c1ccc(OC)cc1] | >50 | >50 | 8.80 |
| 21 | 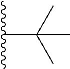 [SMILES: *C(C)(C)C] | 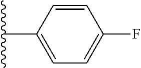 [SMILES: *c1ccc(F)cc1] | >50 | 40.5 | 24.4 |
| 22 | 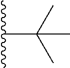 [SMILES: *C(C)(C)C] | 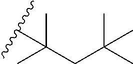 [SMILES: *C(C)(C)CC(C)(C)C] | 1.88 | 1.49 | 1.24 |
| 23 | 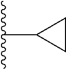 [SMILES: *C1CC1] | 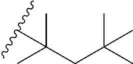 [SMILES: *C(C)(C)CC(C)(C)C] | >50 | >50 | 5.49 |
| 24 | 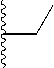 [SMILES: *CC] | 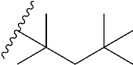 [SMILES: *C(C)(C)CC(C)(C)C] | >50 | >50 | 27.9 |
| 25 | 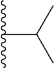 [SMILES: *C(C)C] | 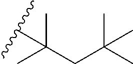 [SMILES: *C(C)(C)CC(C)(C)C] | 3.63 | 10.6 | 7.79 |
| 26 | 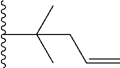 [SMILES: *C(C)(C)CC=C] | 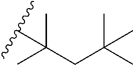 [SMILES: *C(C)(C)CC(C)(C)C] | 9.95 | 8.29 | 1.37 |
| 27 | 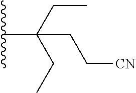 [SMILES: *C(CC)(CC)CCC#N] | 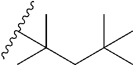 [SMILES: *C(C)(C)CC(C)(C)C] | 11.6 | 18.0 | 14.8 |

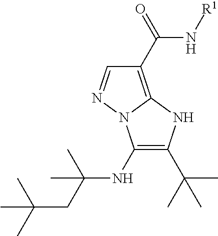

| Example | R¹ | 4T1^{a)} | MCF-7^{a)} | HL-60^{a)} |
|---|---|---|---|---|
| 28 | Me | 4.66 | 4.46 | 1.52 |
| 29 | 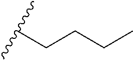 [SMILES: *CCCC] | >50 | 34.1 | 2.72 |
| 31 | 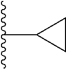 [SMILES: *C1CC1] | 4.01 | 1.69 | 1.61 |
| 33 | 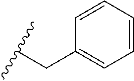 [SMILES: *Cc1ccccc1] | 9.31 | 8.57 | 6.26 |
| 34 | 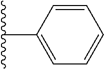 [SMILES: *c1ccccc1] | 28.5 | 18.3 | 0.604 |
| 35 | 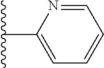 [SMILES: *c1ccccn1] | 6.91 | 8.35 | 4.38 |
| 36 | 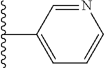 [SMILES: *c1cccnc1] | 2.59 | 1.90 | 0.934 |
| 37 | 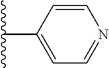 [SMILES: *c1ccncc1] | 4.34 | 2.55 | 2.71 |
| 38 | 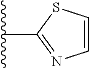 [SMILES: *c1nccs1] | 2.41 | 1.95 | 2.86 |
| 39 | 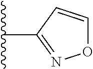 [SMILES: *c1ccon1] | 2.85 | 1.70 | 0.935 |

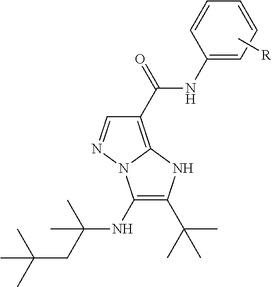

| Example | R | 4T1^{a)} | MCF-7^{a)} | HL-60^{a)} |
|---|---|---|---|---|
| 40 | 2-Me | >50 | 30.6 | 2.12 |
| 43 | 4-OMe | 24.7 | 17.8 | 1.11 |
| 46 | 3-CF₃ | 35.4 | >50 | 11.4 |
| 47 | 4-CF₃ | >50 | 29.0 | >50 |
| 48 | 2-F | >50 | >50 | 2.04 |
| 49 | 3-F | 23.5 | 17.3 | 4.47 |
| 50 | 4-F | 7.43 | 2.35 | 0.183 |
| 51 | 4-Cl | 30.7 | 29.3 | 3.05 |
| 52 | 4-Br | >50 | >50 | 19.7 |
| 53 | 4-NO₂ | 27.1 | 9.94 | 7.64 |
| 54 | 4-CN | >50 | 9.71 | 1.50 |
| 55 | 4-COOEt | >50 | >50 | 8.50 |
| 56 | 4-SMe | >50 | 21.9 | 3.93 |
| 57 | 4-NMe₂ | 11.8 | 10.0 | 0.297 |
| 58 | 2,4-F | >50 | >50 | 0.628 |
| 59 | 3,4-F | 19.0 | 10.3 | 2.41 |

^{a)}IC₅₀ values in μM. Bold values represent compounds displaying an IC₅₀ <1 μM in HL-60 cell line.

### Example 103

### Compounds Described in Example 22, 60 and 83 (DU192, DU283 and DU325) Compromise the Viability of HL-60 Cells, but Human Primary Fibroblast are Resistant to Treatment In Vitro.

To obtain primary human fibroblasts healthy volunteers (age 18-60 years) were enrolled into the study. The punch biopsies were taken from healthy subjects from the breast area undergoing plastic surgery. Primary fibroblasts were obtained from the skin by enzymatic digestion according to a standard protocol. Skin specimens were first washed in Salsol A solution (Human Rt, Gödöllö, Hungary) supplemented with 2% antibiotic/antimycotic solution (Sigma-Aldrich). Skin samples were then cut into narrow strips and incubated in Dispase solution (Roche Diagnostics, Mannheim, Germany) overnight at 4° C. The epidermis was subsequently separated from the dermis. Fibroblasts were obtained by incubating the dermis in Digestion Mix solution (Collagenase, Hyaluronidase and Deoxyribonuclease) for 2h at 37° C. Cell suspensions were filtered through a 100 μm nylon mesh (BD Falcon, San Jose, Calif., USA) and cells were pelleted by centrifugation. Fibroblasts were grown in low glucose DMEM medium containing 5% FBS, 1% antibiotic/antimycotic (PAA, Pasching, Austria) and 1% L-glutamine solution (PAA). Fibroblasts were cultured at 37° C. and 5% CO₂ in humidified conditions. Depending on the cell growth, the medium was changed every 2-4 days and cells were passaged at 80% of confluence.

The human primary fibroblasts (6000 cells/well) and Hl60 cells (20.000 cells/well) were seeded into 96-well plates (Corning Life Sciences) in media. Fibroblasts were cultured overnight before treatment. Effects of compounds described in Example 22, 60 and 83 were examined in concentrations of 1 μM, 250 nM, 62.5 nM, 15.6 nM, 3.9 nM and 0.9 nM in 100 μl after 72 h incubation. Resazurin reagent was prepared and used as described in Example 1.

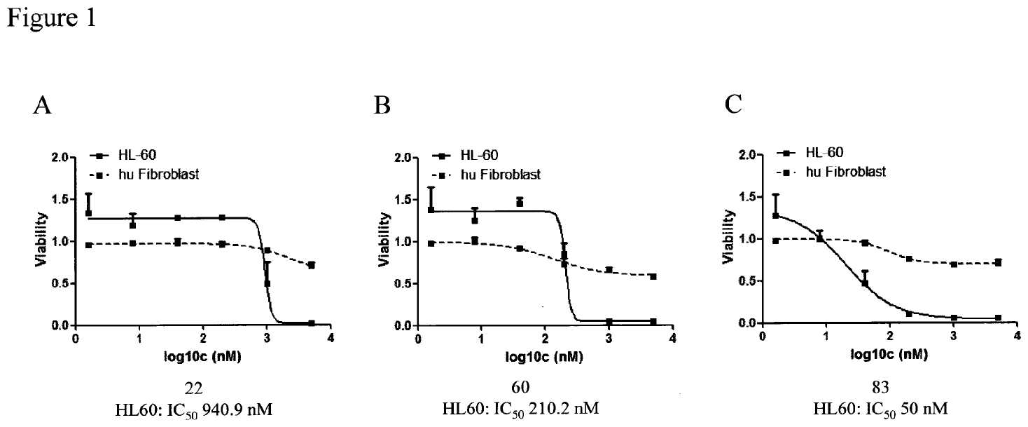

Viability was calculated with relation to untreated control cells and blank wells containing media without cells. IC₅₀ values (50% inhibiting concentration) were calculated by GraphPad Prism® 5. Results are summarized in FIG. 1.

Compounds described in Example 22 (FIG. 1. A), 60 (FIG. 1. B) and 83 (FIG. 1. C) dose dependently decreased the viability of HL-60 cells with half-inhibitory concentration (IC₅₀) values of: 940 nM, 210 nM and 50 nM, respectively. Significant decrease in viability was not apparent for human primary fibroblasts in the applied concentration range (1.6 nM-5 μM), therefore IC₅₀ values could not be determined.

### Example 104

### Compounds Described in Example 60 and 83 Drive Survival Pathways as an Early Response to Treatment in HL-60 Cells.

Human promyelocytic leukemia HL60 Cells (500,000/well) were plated in 24-well tissue culture plates (Corning Life Sciences) in RPMI 10% FCS (Gibco) and were treated with the compounds described in Example 60 and 83 at 40 nM, 200 nM and 1 μM concentrations in 500 μl media. After 24 h incubation time cells with the corresponding supernatants were harvested and centrifuged down (2000 rpm, 5 min).

Pellets were resuspended and fixed in 3.5% PBS buffered formaldehyde (Molar Chemicals) for 10 minutes. Cells were washed with FACS-buffer (2% FCS, (Gibco) in PBS), centrifuged down (2000 rpm, 5 min). Cells were permeabilized in Permeability buffer (1% FCS, 0.1% saponin (Sigma-Aldrich) in PBS pH 7.4) for 5 minutes. Cells were washed with FACS buffer (2% FCS, (Gibco) in PBS), centrifuged (2000 rpm, 5 min). The following primary antibodies were used: Bcl-xl-Alexa 488, (Cell Signaling, cat. numb. 2767S), dilution 1:75, 24 h; pAkt-Alexa Fluor 488, (Ser473), (Cell Signaling, cat. numb, 4071), dilution 1:50, 24 or 72h; After incubation for 1h at 4° C. samples were washed two times with FACS buffer. After washing, 300 μl FACS buffer was added for acquisition with the FACSCalibur flow cytometer using the FL1 channel and CellQuest™ software (Becton Dickinson) acquiring 20.000 events. In order to calculate the signal to noise ratio mean fluorescent intensity (MFI) was calculated by the following equation: =POWER(10; (Median_{stained}−Median _{unstained, untreated})/Chd), where Chd means 256, the number of channels per decade (Fajka-Boja et al. 2002; Sharrow 2001) in Microsoft Excel. Column charts were created by GraphPad Prism® 5.

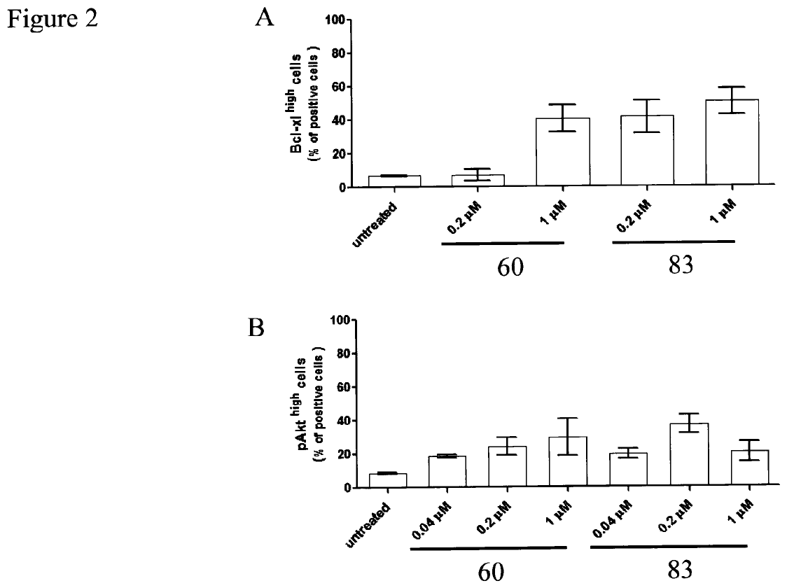

FIG. 2 shows the determined increase of the percentage of the Bcl-xl^{bright} (Figure. 2. A) and pAkt^{bright} cells (FIG. 2. B). Treatment with each compound substantially increased the fraction of cells highly expressing Bel-xl and pAkt indicating an activation of survival pathways.

### Example 105

### The Compound Described in Example 83 Drives the Differentiation of HL-60 Promyelocytes.

HL-60 cells (10×10⁶) were plated in 100 mm tissue culture dishes (Corning Life Sciences) in RPMI 10% FCS. Cells were treated in 10 mL total volume with the compound described in Example 83 24 h after treatment, nucleic acid preparation was done by using the Bioneer RNA purification kit (Bioneer, Viral RNA extraction kit, Daejeon, South Korea) according to an already published protocol (Szebeni et al. 2017a). The quality and quantity of the isolated RNA were measured with NanoDrop1000 Version 3.8.1. (Thermo Fisher Scientific). Reverse transcription from 3 μg of total RNA was performed with the High-Capacity cDNA Archive Kit (Applied Biosystems, Foster City, Calif., USA) in a total volume of 30 μL according to the manufacturer's protocol. Quantitative-real time PCR was carried out using gene specific primers for CD33 (primer sequences: forward 5′ ctgacctgctctgtgtcctg 3′, reverse 5′ atgagcaccgaggagtgagt 3′) and CD34, (primer sequences: forward 5′ gcgctttgcttgctgagt 3′, reverse 5′ gggtagcagtaccgttgttgt 3′) using Sybr Green detection on a LightCycler Nano instrument (Roche, Hungary). Relative gene expression data was normalized to ACTB (beta actin, primer sequences: forward 5′ attggcaatgagcggttc 3′, reverse 5′ cgtggatgccacaggact 3′) expression.

Experiments detecting CD11b expression by Flow cytometric immunofluorescence were done as described in Example 104 without fixation and permeabilization. Native cell surface staining was done by CD11b-FITC (Immunotools cat number 21389113), with 1:20 dilution 24, 48 and 72 h after treatment.

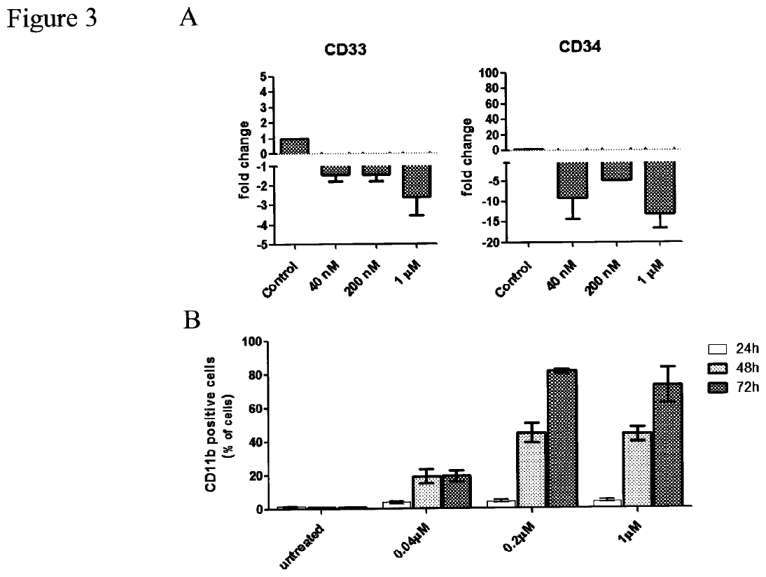

Results are shown in FIG. 3. As a proof of cellular differentiation the expression of haematopoietic stem cell markers CD33 and CD34 decreased following treatment with the compound described in Example 83 (FIG. 3. A). The induced differentiation was further confirmed by the elevation of matured myeloid cell marker, CD11b on the cell surface detected by flow cytometry (FIG. 3. B).

### Example 106. Compounds Described in Example 60 and 83 Differentiation of Promyelocytic Leukemic Cells is Followed by Apoptotic Cell Death

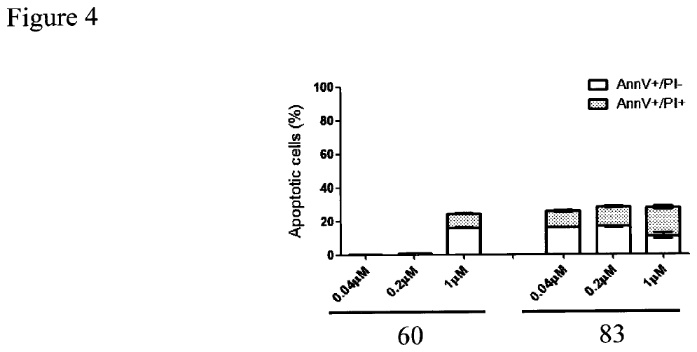

Cells (200,000/well) were plated in 24-well tissue culture plates (Corning Life Sciences) and treated with the compounds described in Example 60 and 83 at 40 nM, 200 nM and 1 μM concentrations in 500 μl media. After 24 h cells were harvested with the corresponding supernatant and centrifuged down (2000 rpm, 5 min). Pellets were resuspended in Annexin V binding buffer (0.01 M HEPES, 0.14 M NaCl and 2.5 mM CaCl₂). Annexin V-Alexa Fluor® 488 (Life Technologies, 2.5:100) was added to the cells, which were then kept for 15 min in the dark at room temperature. Before the acquisition propidium iodide (10 μg/ml, Sigma-Aldrich) was added in Annexin V binding buffer to dilute Annexin V-Alexa Fluor® 488 5×. Cells (20.000 events) were analyzed on a FACSCalibur flow cytometer using CellQuest software (Becton Dickinson). The percentage of the FL1 (530/30 nm filter, Annexin V-Alexa Fluor® 488) positive and FL3 (670 nm filter, propidium iodide) negative early apoptotic cells and FL1 positive and FL3 positive late apoptotic cells were determined. The total apoptotic population included both early and late apoptotic cells. Column charts were created by GraphPad Prism® 5. Results are depicted in FIG. 4. In these experiments we investigated whether treatment of HL-60 cells resulted in phosphatidylserine exposure as a sign of the induction of programmed cell death. It is apparent that treatment induced differentiation of HL-60 cells was accompanied by apoptosis. We could detect AnnexinV⁺/PI⁻ early and AnnexinV⁺/PI⁺ late apoptotic cell populations after 24 h of treatment.

### Example 107. Compounds Described in Example 60 and 83 Induce Caspase-3 Activation in HL-60 Cells

Detection of caspase-3 activation by flow cytometric immunofluorescence was done as described in Example 104 with the exception of the used antibodies. Rabbit polyclonal caspase-3 antibody (Cell Signaling, unconjugated, cat numb. 9661S) was added in 1:600 dilution in FACS buffer. After incubation for 1h at 4° C. samples were washed two times with FACS buffer. The secondary antibody for anti-caspase-3, anti-rabbit IgG conjugated with Alexa Fluor® 488 (Thermo Fisher Scientific, A11008) was diluted to 1:600 and incubated with the cells for 30 min at 4° C.

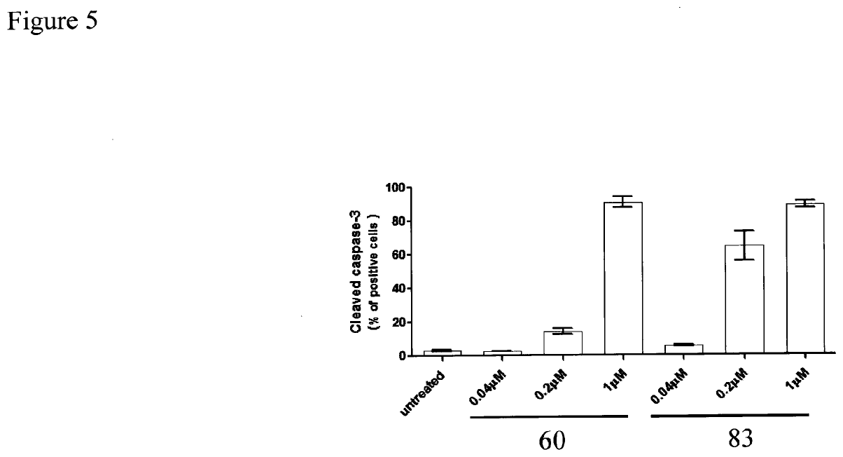

Treatment with compounds described in Example 60 and 83 increased the percentage of active caspase-3 positive cells (FIG. 5) providing evidence for the activation of the caspase-3 dependent apoptotic cascade leading to cell death.

### Example 108

### In Vitro Citotoxic Effects of Compounds Described in Example 22, 60 and 83 in Different Cell Lines

All cell lines were purchased from the American Type Culture Collection (ATCC, Manassas, Va., USA).

GBM2 (human glioblastoma), HeLa (human cervical carcinoma), MIA PaCa-2 (human pancreas carcinoma) and U87MG (human glioblastoma) cells were maintained in Dulbecco's Modified Eagle Medium (DMEM) 10% fetal calf serum (FCS, Gibco). A375 (human melanoma), A549 (human lung adenocarcinoma) and HEP3B (human hepatoma) cells were maintained in Dulbecco's Modified Eagle Medium/Nutrient Mixture F-12 (DMEM/F12) 10% FCS.

HT168 (human melanoma), HT199 (human melanoma), HT29 (human colorectal adenocarcinoma), MOLT4 (human leukemia) and U937 (human lymphoma) cells were maintained in Roswell Park Memorial Institute 1640 medium (RPMI-1640) 10% FCS. SKOV-3 cells were maintained in Dulbecco's Modified Eagle Medium/McCoy's medium (DMEM/McCoy) 10% FCS.

Media were supplemented with 2 mM GlutaMAX, and 100 U/mL penicillin, 100 μg/mL streptomycin (Life Technologies, Carlsbad, Calif., USA). Cell cultures were maintained at 37° C. in a humidified incubator in an atmosphere of 5% CO₂ (Sanyo, Japan).

Viability assays were performed as described in Example 102. Calculated IC₅₀ (μM) values are listed in Table 2. The selected compounds exhibited potent cytotoxic activity against all tested cell types.

TABLE 2
in vitro citotoxic effects of compounds described in
Example 22, 60 and 83 (IC₅₀, μM) on various cell lines

| | | compound | compound | compound |
|---|---|---|---|---|
| cell type | disease | 22 | 60 | 83 |
| HeLa | cervical carcinoma | | | 0.1 |
| HT29 | colorectal | | 0.25 | 0.05 |
| | adenocarcinoma | | | |
| GBM2 | glioblastoma | 5 | 0.25 | 0.1 |
| U87 | glioblastoma | | 0.25 | |
| HEP3B | hepatoma | | 0.25 | |
| MOLT4 | leukemia | | | 0.15 |
| U937 | lymphoma | | | 0.25 |
| A549 | lung adenocarcinoma | | 0.75 | 0.3 |
| A375 | melanoma | 2 | | |
| HT168 | melanoma | 3 | 0.5 | 0.1 |
| HT199 | melanoma | | 0.3 | 0.1 |
| SKOV-3 | ovarian | | | 0.15 |
| | adenocarcinoma | | | |
| MIA PaCa- | pancreas carcinoma | | 0.3 | 0.3 |
| 2 | | | | |

### Example 109

### The Anticancerous Effect of Compound 83 in Live Animals: I. Mammary Carcinoma

The effect on mammary carcinoma was studied on BalbC mouse model inoculated subcutaneously into the mammary gland with 4T1 mouse cells (ATCC) (100,000 cells/animal). Two groups were formed from randomly selected mice, with 8 animals in both groups. Group 1: control group, it was only administered a carrier (0.1 mL, 0.9% NaCl solution) intravenously; group 2: group treated with compound 83, it was administered 3 mg/kg of compound 83 in PEG100:Solutol:PBS (1:4:15 vol ratio), intravenously after the tumor reached 300 mm³ (day 16).

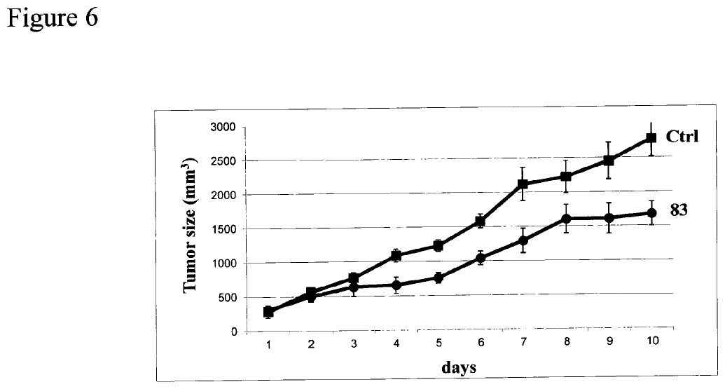

The treatments were performed from the sixteenth day, every other day, for a total of 6 occasions. Starting from the 16^{th} day on every day the size of the increasing tumours was determined in the case of each animal, and the group average was represented per group (FIG. 6). The standard deviation was determined in SEM. It can be seen that the treatment with compound 83 reduced the size of the increasing mammary tumour.

### Example 110

### The Anticancerous Effect of Compound 60 in Live Animals: II. Leukaemia

The effect on leukaemia was studied on SCID immune-deficient mouse model inoculated intravenously with HL60 human acute myeloid leukaemia cells (ATCC) (1 million cells/animal). Two groups were formed from randomly selected mice, with 9 animals in each group. Group1: control group, it was only administered a carrier (0.1 mL, 0.9% NaCl solution) intravenously; group 2: group treated with compound 60, it was administered 3 mg/kg of compound 60 in PEG100:Solutol:PBS (1:4:15 vol ratio), intravenously.

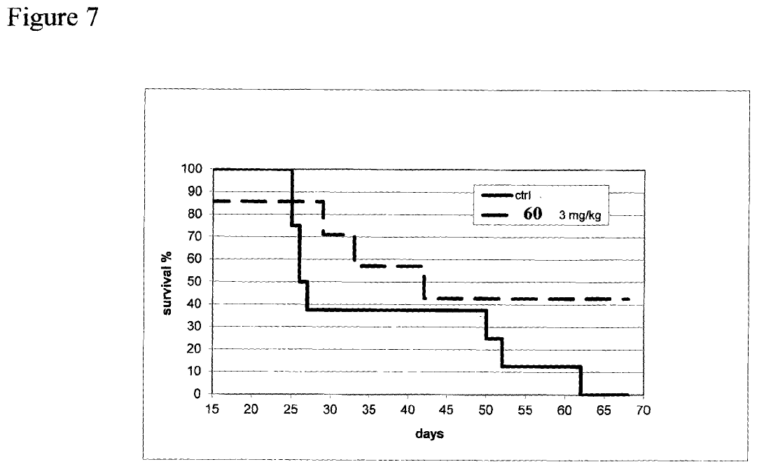

The treatments were performed from the third day, on five consecutive occasions per week, for 2 weeks, on a total of 10 occasions. As time went on, every day we determined the number of surviving animals and represented it in percentage per group as compared to the total initial number of animals (FIG. 7). It can be seen that the 3 mg/kg dose of compound 60 administered intravenously was effective, the treatment with compound 60 increased the LD50 (from day 26 to day 42) and the survival rate of the animals.

### Example 111

### The Anticancerous Effect of Compound 83 in Live Animals: III. Melanoma

The effect on melanoma was studied on SCID immune-deficient mouse model inoculated in the spleen with HTT199 human melanoma cells (ATCC) (1 million cells/animal). Two groups were formed from randomly selected mice, with 10 animals in each group. Group 1: control group, it was only administered a carrier (0.1 mL, 0.9% NaCl solution) intravenously; group 2: group treated with compound 83, it was administered 3 mg/kg of compound 83 in PEG100:Solutol:PBS (1:4:15 vol ratio), intravenously.

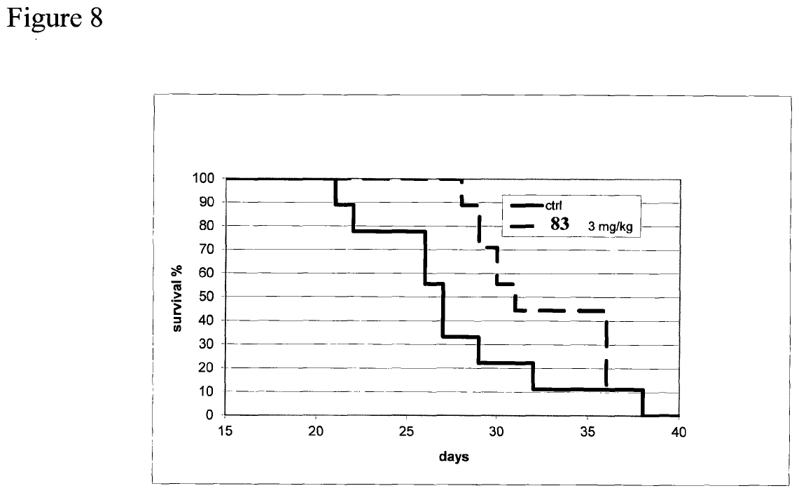

The treatments were performed from the third day, on five consecutive occasions per week, for 2 weeks, on a total of 10 occasions. As time went on, every day we determined the number of surviving animals and represented it in percentage per group as compared to the total initial number of animals (FIG. 8). It can be seen that the 3 mg/kg dose of compound 83 administered intravenously was effective, the treatment with compound 83 increased the LD50 (from day 33 to day 38) and the survival rate of the animals.

### Example 112

### In Vitro Citotoxic Effects of the Compound Described in Example 90 in Different Cell Lines

Human primary fibroblasts were obtained from the skin by enzymatic digestion according to a standard protocol. Fibroblasts were grown in low glucose DMEM/F12 medium containing 15% FCS, 1% antibiotic/antimycotic (PAA, Pasching, Austria) and 1% L-glutamine solution (PAA). Fibroblasts were cultured at 37° C. and 5% CO₂ in humidified conditions. Depending on the cell growth, the medium was changed every 2-4 days and cells were passaged at 80% of confluence.

Cancer cell lines were purchased from the American Type Culture Collection (ATCC, Manassas, Va., USA). HT29 (human colorectal adenocarcinoma), HL-60 (acute promyelocytic leukemia), THP-1 (acute monocytic leukemia), MOLT-4 (acute T-lymphoblastic leukemia), MV-4-11 (biphenotypic B myelomonocytic leukemia) and K-562 (erythroleukemia) cells were maintained in Roswell Park Memorial Institute 1640 medium (RPMI-1640) with 10% FCS. Media were supplemented with 2 mM GlutaMAX, and 100 U/mL penicillin, 100 μg/mL streptomycin (Life Technologies, Carlsbad, Calif., USA). Cell cultures were maintained at 37° C. in a humidified incubator in an atmosphere of 5% CO₂ (Sanyo, Japan).

Viability assays were performed as described in Example 102 with minor modification for cell density and tested concentration range. Applied cell densities: in case of human primary fibroblast 6000, for HT29 4000, for HL-60, MOLT-4, MV-4-11, THP-1, K-562 20000 cells/well. Applied compound concentration range: 10 μM-0.2 nM. Calculated IC₅₀ (nM) values are listed in Table 3. The selected compounds exhibited potent cytotoxic activity against all tested cell types.

TABLE 3
in vitro citotoxic effects of the compound described
in Example 90 (IC₅₀, nM) on various cell lines

| cell type | disease | compound 90 |
|---|---|---|
| HT-29 | colorectal | 9.97 |
| | adenocarcinoma | |
| HL-60 | promyelocytic leukemia | 16.54 |
| MOLT-4 | acute T-lymphoblastic | 27.24 |
| | leukemia | |
| MV-4-11 | biphenotypic B | 32.25 |
| | myelomonocytic | |
| | leukemia | |
| THP-1 | acute monocytic | 25.88 |
| | leukemia | |
| K-562 | erythroleukemia | 54.31 |
| human primary | — | >3000 |
| fibroblast | | |

### REFERENCES

Baviskar A T, Madaan C, Preet R, Mohapatra P, Jain V, Agarwal A, Guchhait S K, Kundu C N, Banerjee U C, Bharatam P V. N-fused imidazoles as novel anticancer agents that inhibit catalytic activity of topoisomerase IIa and induce apoptosis in Gl/S phase. J. Med. Chem. 2011, 54, 5013-5030.
Bindi S, Fancelli D, Alli C, Berta D, Bertrand J A, Cameron A D, Cappella P, Carpinelli P, Cervi G, Croci V, D'Anello M, Forte B, Laura Giorgini M, Marsiglio A, Moll J, Pesenti E, Pittali V, Pulici M, Riccardi-Sirtori F, Roletto F, Soncini C, Storici P, Varasi M, Volpi D, Zugnoni P, Vianello P. Thieno[3,2-c]pyrazoles: A novel class of Aurora inhibitors with favorable antitumor activity Bioorg. Med. Chem. 2010, 18, 7113-7120.
Bondock S, Adel S, Etman H A, Badria F A. Synthesis and antitumor evaluation of some new 1,3,4-oxadiazole-based heterocycles. Eur. J. Med. Chem. 2012, 48, 192-199.
De Kouchkovsky I, Abdul-Hay M (2016) Acute myeloid leukemia: a comprehensive review and 2016 update. Blood cancer journal 6:e441 doi:10.1038/bcj.2016.50
Demjén A, Gyuris M, Wölfling J, Puskas L G, Kanizsai I. Facile synthesis of 1H-imidazo[1,2-b]pyrazoles via a sequential one-pot synthetic approach. Beilstein J. Org. Chem. 2014, 10, 2338-2344.
Dinér P, Alao J P, Söderlund J, Sunnerhagen P, Grotli M. Preparation of 3-substituted-1-isopropyl-1H-pyrazolo[3,4-d]pyrimidin-4-amines as RET kinase inhibitors. J. Med. Chem. 2012, 55, 4872-4876.
Dwyer M P, Paruch K, Labroli M, Alvarez C, Keertikar K M, Poker C, Rossman R, Fischmann T O, Duca J S, Madison V, Parry D, Davis N, Seghezzi W, Wiswell D, Guzi T J., Discovery of pyrazolo[1,5-a]pyrimidine-based CHK1 inhibitors: a template-based approach—Part 1. Bioorg. Med. Chem. Lett. 2011, 21, 467-470.
El-borai M A, Rizk H F, Abd-Aal M F, El-Deeb I Y. Synthesis of pyrazolo[3,4-b]pyridines under microwave irradiation in multi-component reactions and their antitumor and antimicrobial activities—Part 1. Eur. J. Med. Chem. 2012, 48, 92-96.
Fajka-Boja R et al. (2002) Fermented wheat germ extract induces apoptosis and downregulation of major histocompatibility complex class I proteins in tumor T and B cell lines International journal of oncology 20:563-570
Gangat N, Patnaik M M, Tefferi A (2016) Myelodysplastic syndromes: Contemporary review and how we treat American journal of hematology 91:76-89 doi:10.1002/ajh.24253
Germing U, Kobbe G, Haas R, Gattermann N (2013) Myelodysplastic syndromes: diagnosis, prognosis, and treatment Deutsches Arzteblatt international 110:783-790 doi:10.3238/arztebl.2013.0783
Grosse S, Mathieu V, Pillard C, Massip S, Marchivie M, Jarry C, Bernard P, Kiss R, Guillaumet G. New imidazo[1,2-b]pyrazoles as anticancer agents: synthesis, biological evaluation and structure activity relationship analysis. Eur. J. Med. Chem. 2014, 84, 718-730.
Guo Y, Wang Z. (Beigene, Ltd). W O 2014/173289 A1. 2014.
Hanan E J, van Abbema A, Barrett K, Blair W S, Blaney J, Chang C, Eigenbrot C, Flynn S, Gibbons P, Hurley C A, Kenny J R, Kulagowski J, Lee L, Magnuson S R, Morris C, Murray J, Pastor R M, Rawson T, Siu M, Ultsch M, Zhou A, Sampath D, Lyssikatos J P., Discovery of potent and selective pyrazolopyrimidine janus kinase 2 inhibitors. J. Med. Chem. 2012, 55, 10090-10107.
Hu G Q, Hou L L, Yang Y, Yi L, Xie S Q, Wang G Q, Duan N N, Chao T Y, Wen X Y, Huang W L. Synthesis and antitumor evaluation of fluoroquinolone C3 fused heterocycles (I I): From triazolothiadiazines to pyrazolotriazoles Chin. Chem. Lett. 2011, 22, 804-806.
Janols H, Bergenfelz C, Allaoui R, Larsson A M, Ryden L, Bjornsson S, Janciauskiene S, Wullt M, Bredberg A, Leandersson K: A high frequency of MDSCs in sepsis patients, with the granulocytic subtype dominating in gram-positive cases. Journal of leukocyte biology 2014, 96(5):685-693.
Kawamata H, Tachibana M, Fujimori T, Imai Y. (2006) Differentiation-inducing therapy for solid tumors. Curr Pharm Des. 12:379-385.
Kim H, Kim M, Lee J, Yu H, Hah J M. Syntheses of phenylpyrazolodiazepin-7-ones as conformationally rigid analogs of aminopyrazole amide scaffold and their antiproliferative effects on cancer cells. Bioorg. Med. Chem. 2011, 19, 6760-6767.
Kosugi T, Mitchell D R, Fujino A, Imai M, Kambe M, Kobayashi S, Makino H, Matsueda Y, Oue Y, Komatsu K, Imaizumi K, Sakai Y, Sugiura S, Takenouchi O, Unoki G, Yamakoshi Y, Cunliffe V, Frearson J, Gordon R, Harris C J, Kalloo-Hosein H, Le J, Patel G, Simpson D J, Sherborne B, Thomas P S, Suzuki N, Takimoto-Kamimura M, Kataoka K. Mitogen-activated protein kinase-activated protein kinase 2 (MAPKAP-K2) as an antiinflammatory target: discovery and in vivo activity of selective pyrazolo[1,5-a]pyrimidine inhibitors using a focused library and structure-based optimization approach. J. Med. Chem. 2012, 55, 6700-6715.
Labroli M, Paruch K, Dwyer M P, Alvarez C, Keertikar K, Poker C, Rossman R, Duca J S, Fischmann T O, Madison V, Parry D, Davis N, Seghezzi W, Wiswell D, Guzi T J. Discovery of pyrazolo[1,5-a]pyrimidine-based CHK1 inhibitors: a template-based approach—part 2. Bioorg. Med. Chem. Lett. 2011, 21, 471-474.
Lathers D M, Clark J I, Achille N J, Young M R (2004) Phase 1B study to improve immune responses in head and neck cancer patients using escalating doses of 25-hydroxyvitamin D3 Cancer immunology, immunotherapy: CII 53:422-430 doi:10.1007/s00262-003-0459-7
Le Brazidec J Y, Pasis A, Tam B, Boykin C, Black C, Wang D, Claassen G, Chong J H, Chao J, Fan J, Nguyen K, Silvian L, Ling L, Zhang L, Choi M, Teng M, Pathan N, Zhao S, Li T, Taveras A. Synthesis, SAR and biological evaluation of 1,6-disubstituted-1H-pyrazolo[3,4-d]pyrimidines as dual inhibitors of Aurora kinases and CDK1. Bioorg. Med. Chem. Lett. 2012, 22, 2070-2074.
Lee M, Park C S, Lee Y R, Im S A, Song S, Lee C K (2014) Resiquimod, a TLR7/8 agonist, promotes differentiation of myeloid-derived suppressor cells into macrophages and dendritic cells Archives of pharmacal research 37:1234-1240 doi:10.1007/s12272-014-0379-4
Li M, Progress of the synthesis of condensed pyrazole derivatives (from 2010 to mid-2013). Zhao B X, Eur. J. Med. Chem. 2014, 85, 311-340.
Liu Z Q. Two Neglected Multicomponent Reactions: Asinger and Groebke Reaction for Constructing Thiazolines and Imidazolines. Curr. Org. Synth. 2015, 12, 20-60.
Lukasik P M, Elabar S, Lam F, Shao H, Liu X, Abbas A Y, Wang S. Synthesis and biological evaluation of imidazo[4,5-b]pyridine and 4-heteroaryl-pyrimidine derivatives as anti-cancer agents. Eur. J. Med. Chem. 2012, 57, 311-322.
Mathias B, Delmas A L, Ozrazgat-Baslanti T, Vanzant E L, Szpila B E, Mohr A M, Moore F A, Brakenridge S C, Brumback B A, Moldawer L L et al: Human Myeloid-derived Suppressor Cells are Associated With Chronic Immune Suppression After Severe Sepsis/Septic Shock. Annals of surgery 2017, 265(4):827-834
McPeak M B, Youssef D, Williams D A, Pritchett C, Yao Z Q, McCall C E, El Gazzar M: Myeloid Cell-Specific Knockout of NFI-A Improves Sepsis Survival. Infection and immunity 2017, 85(4)
Mirza N et al. (2006) All-trans-retinoic acid improves differentiation of myeloid cells and immune response in cancer patients Cancer research 66:9299-9307 doi:10.1158/0008-5472.CAN-06-1690
Moen M D, McKeage K, Plosker G L, Siddiqui M A (2007) Imatinib: a review of its use in chronic myeloid leukaemia Drugs 67:299-320
Nie Z, Perretta C, Erickson P, Margosiak S, Lu J, Averill A, Almassy R, Chu S. Structure-based design and synthesis of novel macrocyclic pyrazolo[1,5-a][1,3,5]triazine compounds as potent inhibitors of protein kinase CK2 and their anticancer activities. Bioorg. Med. Chem. Lett. 2008, 18, 619-623.
Popowycz F, Foumet G, Schneider C, Bettayeb K, Ferandin Y, Lamigeon C, Tirado O M, Mateo-Lozano S, Notario V, Colas P, Bernard P, Meijer L, Joseph B. Pyrazolo[1,5-a]-1,3,5-triazine as a purine bioisostere: access to potent cyclin-dependent kinase inhibitor (R)-roscovitine analogue. J. Med. Chem. 2009, 52, 655-663.
Radi M, Brullo C, Crespan E, Tintori C, Musumeci F, Biava M, Schenone S, Dreassi E, Zamperini C, Maga G, Pagano D, Angelucci A, Bologna M, Botta M. Identification of potent c-Src inhibitors strongly affecting the proliferation of human neuroblastoma cells. Bioorg. Med. Chem. Lett. 2011b, 21, 5928-5933.
Radi M, Dreassi E, Brullo C, Crespan E, Tintori C, Bernardo V, Valoti M, Zamperini C, Daigl H, Musumeci F, Carraro F, Naldini A, Filippi I, Maga G, Schenone S, Botta M. Design, synthesis, biological activity, and ADME properties of pyrazolo[3,4-d]pyrimidines active in hypoxic human leukemia cells: a lead optimization study. Med. Chem. 2011a, 54, 2610-2626.
Raffa D, Maggio B, Raimondi M V, Cascioferro S, Plescia F, Cancemi G, Daidone G. Recent advanced in bioactive systems containing pyrazole fused with a five membered heterocycle. Eur. J. Med. Chem. 2015, 97, 732-746.
Ren L, Laird E R, Buckmelter A J, Dinkel V, Gloor S L, Grina J, Newhouse B, Rasor K, Hastings G, Gradl S N, Rudolph J. Potent and selective pyrazolo[1,5-a]pyrimidine based inhibitors of B-Raf(V600E) kinase with favorable physicochemical and pharmacokinetic properties. Bioorg. Med. Chem. Lett. 2012, 22, 1165-1168.
Shaaban M R, Saleh T S, Mayhoub A S, Farag A M. Single step synthesis of new fused pyrimidine derivatives and their evaluation as potent Aurora-A kinase inhibitors. Eur. J. Med. Chem. 2011, 46, 3690-3695.
Shaaban S, Abdel-Wahab B F. Groebke-Blackburn-Bienaymé multicomponent reaction: emerging chemistry for drug discovery. Mol. Divers. 2016, 20, 233-254.
Sharrow S O (2001) Analysis of flow cytometry data Current protocols in immunology Chapter 5: Unit 5 2 doi:10.1002/0471142735.im0502s00
Sica A, Erreni M, Allavena P, Porta C (2015) Macrophage polarization in pathology Cellular and molecular life sciences: CMLS 72:4111-4126 doi:10.1007/s00018-015-1995-y
Soth M, Abbot S, Abubakari A, Arora N, Arzeno H, Billedeau R, Dewdney N, Durkin K, Frauchiger S, Ghate M, Goldstein D M, Hill R J, Kuglstatter A, Li F, Loe B, McCaleb K, McIntosh J, Papp E, Park J, Stahl M, Sung M L, Suttman R, Swinney D C, Weller P, Wong B, Zecic H, Gabriel T. 3-Amino-pyrazolo[3,4-d]pyrimidines as p38 α kinase inhibitors: design and development to a highly selective lead. Bioorg. Med. Chem. Lett. 2011, 21, 3452-3456.
Staben S T, Heffron T P, Sutherlin D P, Bhat S R, Castanedo G M, Chuckowree I S, Dotson J, Folkes A J, Friedman L S, Lee L, Lesnick J, Lewis C, Murray J M, Nonomiya J, Olivero A G, Plise E, Pang J, Prior W W, Salphati L, Rouge L, Sampath D, Tsui V, Wan N C, Wang S, Weismann C, Wu P, Zhu B Y. Structure-based optimization of pyrazolo-pyrimidine and -pyridine inhibitors of PI3-kinase. Bioorg. Med. Chem. Lett. 2010, 20, 6048-6051.
Strauss L et al. (2015) RORC1 Regulates Tumor-Promoting “Emergency” Granulo-Monocytopoiesis Cancer cell 28:253-269 doi:10.1016/j.ccell.2015.07.006
Szebeni G J et al. (2017a) Achiral Mannich-Base Curcumin Analogs Induce Unfolded Protein Response and Mitochondrial Membrane Depolarization in PANC-1 Cells. International journal of molecular sciences 18 doi:10.3390/ijms18102105
Szebeni G J, Vizler C, Kitajka K, Puskas L G (2017b) Inflammation and Cancer: Extra- and Intracellular Determinants of Tumor-Associated Macrophages as Tumor Promoters Mediators of inflammation 2017:9294018 doi:10.1155/2017/9294018
Szebeni G J, Vizler C, Nagy L I, Kitajka K, Puskas L G (2016) Pro-Tumoral Inflammatory Myeloid Cells as Emerging Therapeutic Targets International journal of molecular sciences 17 doi:10.3390/ijmsl7111958
Tefferi A, Pardanani A (2015) Myeloproliferative Neoplasms: A Contemporary Review JAMA oncology 1:97-105 doi:10.1001/jamaoncol.2015.89
Vardiman J W et al. (2009) The 2008 revision of the World Health Organization (WHO) classification of myeloid neoplasms and acute leukemia: rationale and important changes Blood 114:937-951 doi:10.1182/blood-2009-03-209262
Wang G T, Mantei R A, Hubbard R D, Wilsbacher J L, Zhang Q, Tucker L, Hu X, Kovar P, Johnson E F, Osterling D J, Bouska J, Wang J. Davidsen S K, Bell R L, Sheppard G S. Substituted 4-amino-1H-pyrazolo[3,4-d]pyrimidines as multi-targeted inhibitors of insulin-like growth factor-1 receptor (IGF1R) and members of ErbB-family receptor kinases. Bioorg. Med. Chem. Lett. 2010, 20, 6067-6071.
Wang Z, Guo Y. (BeiGene, Ltd). US 2017/0073349 A1. 2017.
Wesolowski R, Markowitz J, Carson W E, 3rd (2013) Myeloid derived suppressor cells—a new therapeutic target in the treatment of cancer Journal for immunotherapy of cancer 1:10 doi:10.1186/2051-1426-1-10
Yang L L, Li G B, Yan H X, Sun Q Z, Ma S, Ji P, Wang Z R, Feng S, Zou J, Yang S Y. Discovery of N6-phenyl-1H-pyrazolo[3,4-d]pyrimidine-3,6-diamine derivatives as novel CK1 inhibitors using common-feature pharmacophore model based virtual screening and hit-to-lead optimization. Eur. J. Med. Chem. 2012, 56, 30-38.
Yu H, Jung Y, Kim H, Lee J, Oh C H, Yoo K H, Sim T, Hah J M. 1,4-dihydropyrazolo[4,3-d]imidazole phenyl derivatives: a novel type II Raf kinase inhibitors. Bioorg. Med. Chem. Lett. 2010, 20, 3805-3808.
Zhang J, Singh R, Goff D, Kinoshita T. US 2010/0316649 A1. 2010.

1. Novel bicyclic imidazo[1,2-b]pyrazole carboxamide and carbothioamide derivatives and pharmaceutically acceptable salts thereof 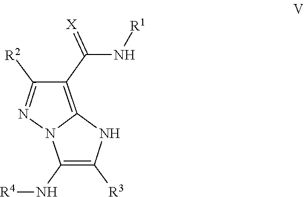
wherein in general formula (V)
R₁ represents hydrogen; branched or unbranched C1-C8-alkyl, aralkyl or aryl group;
furthermore represents heteroaryl group and heterocycles in saturated or unsaturated forms containing O, N and/or S atoms;
R₂ represents hydrogen and branched or un-branched C1-C8-alkyl group;
R₃ represents aliphatic branched or unbranched C1-C8-alkyl, aralkyl or aryl group;
furthermore represents heteroaryl group and heterocycles in saturated or unsaturated forms containing O, N and/or S atoms;
R₄ represents aliphatic branched or unbranched C1-C8-alkyl group; CH2R′ group wherein R′ represents hydrogen, branched or unbranched C1-C8 alkyl group; CO(OR″) group, wherein R″ represents branched or unbranched C1-C8 alkyl; aralkyl or aryl group; furthermore represents heteroaryl group and heterocycles in saturated or unsaturated forms containing O, N and/or S atoms; C(O)R′ group, wherein R′ represents heteroaryl group; and
X represents O- or S-atom.

2. The bicyclic imidazo[1,2-b]pyrazole carboxamide derivatives of general formula (IV) and pharmaceutically acceptable salts thereof according to claim 1, 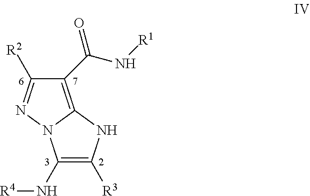
wherein in general formula (IV)
R₁ to R₄ represent the same groups of general formula (V); and
X represents O-atom.

3. The bicyclic imidazo[1,2-b]pyrazole carboxamide derivatives of general formula (IV′) and pharmaceutically acceptable salts thereof according to claim 1, 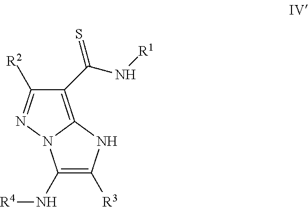
wherein in general formula (IV)
R₁ to R₄ represent the same groups of general formula (V); and
X represents S-atom.

4. The bicyclic imidazo[1,2-b]pyrazole carboxamide or carbothioamide derivatives of general formula (V) and pharmaceutically acceptable salts thereof according to claim 1,
wherein
R₁ represents unsubstituted and substituted phenyl or benzyl group; furthermore represents three-, four-, five-, six- and seven membered heterocyclic ring;
R₃ represents tert-butyl, cyclopentyl, cyclohexyl group; unsubstituted and substituted phenyl or benzyl group; furthermore represents three-, four-, five-, six- and seven membered heterocyclic ring; and
R₄ represents methyl, n-pentyl, 1,1,3,3-tetramethylbutyl, tert-butyl group; CO(OR″) group, wherein R″ represents unsubstituted and substituted phenyl or benzyl group; furthermore represents three-, four-, five-, six- and seven membered heterocyclic ring.

5. The bicyclic imidazo[1,2-b]pyrazole carboxamide or carbothiomide derivatives of general formula (V) and pharmaceutically acceptable salts thereof according to claim 1,
wherein
R₁ represents phenyl or benzyl group substituted with 1; 2; 3; or 4 electron-withdrawing or electron-donating groups in ortho- metha and/or para positions;
R₃ represents phenyl or benzyl group substituted with 1; 2; 3; or 4 electron-withdrawing or electron-donating groups in ortho- metha and/or para positions; and
R₄ represents CO(OR″) group, wherein R″ represents phenyl or benzyl group substituted with 1; 2; 3; or 4 electron-withdrawing or electron-donating groups in ortho- metha and/or para positions.

6. The bicyclic imidazo[1,2-b]pyrazole carboxamide or carbothiomide derivatives of general formula (V) and pharmaceutically acceptable salts thereof according to claim 1,
wherein
R₁ represents unsubstituted phenyl group, 4-fluoro-, 4-N-dimethylamino-, 2,4-difluoro-, 4-aminophenyl, 4-SMe, 4-OH substituted phenyl group;
furthermore represents isoxazole and 3-pyridyl group;
R₂ represents hydrogen;
R₃ represents tert-butyl, 1,1,3,3-tetramethylbutyl, alicyclic cyclohexyl group; and
R₄ represents tert-butyl, 1,1,3,3-tetramethylbutyl and cyclohexyl group.

7. The bicyclic imidazo[1,2-b]pyrazole carboxamide derivatives of general formula (IV) and pharmaceutically acceptable salts thereof according to claim 1 as listed as follows:
Primary carboxamide derivatives and pharmaceutically acceptable salts thereof:
3-(Tert-butylamino)-2-phenyl-1H-imidazo[1,2-b]pyrazole-7-carboxamide;
2-Phenyl-3-((2,4,4-trimethylpentan-2-yl)amino)-1H-imidazo[1,2-b]pyrazole-7-carbox-amide;
Methyl 2-((7-carbamoyl-2-phenyl-1H-imidazo[1,2-b]pyrazol-3-yl)amino)acetate;
3-(Cyclohexylamino)-2-phenyl-1H-imidazo[1,2-b]pyrazole-7-carboxamide;
3-((4-Methoxyphenyl)amino)-2-phenyl-1H-imidazo[1,2-b]pyrazole-7-carboxamide;
2-(p-Tolyl)-3-((2,4,4-trimethylpentan-2-yl)amino)-1H-imidazo[1,2-b]pyrazole-7-carbox-amide;
2-(4-Methoxyphenyl)-3-((2,4,4-trimethylpentan-2-yl)amino)-1H-imidazo[1,2-b]pyrazole-7-carbox-amide;
4-(7-Carbamoyl-3-((2,4,4-trimethylpentan-2-yl)amino)-1H-imidazo[1,2-b]pyrazol-2-yl)-2-methoxy-phenyl acetate;
Methyl 2-((7-carbamoyl-2-(2,4,6-trimethoxyphenyl)-1H-imidazo[1,2-b]pyrazol-3-yl) amino)acetate;
2-(4-Fluorophenyl)-3-((2,4,4-trimethylpentan-2-yl)amino)-1H-imidazo[1,2-b]pyrazole-7-carboxamide;
Methyl 2-((7-carbamoyl-2-(4-fluorophenyl)-1H-imidazo[1,2-b]pyrazol-3-yl)amino) acetate;
2-(4-(Trifluoromethyl)phenyl)-3-((2,4,4-trimethylpentan-2-yl)amino)-1H-imidazo[1,2-b]pyrazole-7-carboxamide;
3-(Tert-butylamino)-2-(3,4-difluorophenyl)-1H-imidazo[1,2-b]pyrazole-7-carboxamide;
2-(Pyridin-3-yl)-3-((2,4,4-trimethylpentan-2-yl)amino)-1H-imidazo[1,2-b]pyrazole-7-carboxamide;
(E)-3-(Tert-butylamino)-2-(1-phenylprop-1-en-2-yl)-1H-imidazo[1,2-b]pyrazole-7-carboxamide;
2-Cyclohexyl-3-((2,4,4-trimethylpentan-2-yl)amino)-1H-imidazo[1,2-b]pyrazole-7-carboxamide;
3-(Tert-butylamino)-2-heptyl-1H-imidazo[1,2-b]pyrazole-7-carboxamide;
2-(Tert-butyl)-3-(cyclohexylamino)-1H-imidazo[1,2-b]pyrazole-7-carboxamide;
2-(Tert-butyl)-3-(tert-butylamino)-1H-imidazo[1,2-b]pyrazole-7-carboxamide;
2-(Tert-butyl)-3-((4-methoxyphenyl)amino)-1H-imidazo[1,2-b]pyrazole-7-carboxamide;
2-(Tert-butyl)-3-((4-fluorophenyl)amino)-1H-imidazo[1,2-b]pyrazole-7-carboxamide;
2-(Tert-butyl)-3-((2,4,4-trimethylpentan-2-yl)amino)-1H-imidazo[1,2-b]pyrazole-7-carbox-amide;
2-Cyclopropyl-3-((2,4,4-trimethylpentan-2-yl)amino)-1H-imidazo[1,2-b]pyrazole-7-carboxamide;
2-Ethyl-3-((2,4,4-trimethylpentan-2-yl)amino)-1H-imidazo[1,2-b]pyrazole-7-carboxamide;
2-Isopropyl-3-((2,4,4-trimethylpentan-2-yl)amino)-1H-imidazo[1,2-b]pyrazole-7-carbox-amide;
2-(2-Methylpent-4-en-2-yl)-3-((2,4,4-trimethylpentan-2-yl)amino)-1H-imidazo[1,2-b]pyrazole-7-carboxamide; and
2-(1-Cyano-3-ethylpentan-3-yl)-3-((2,4,4-trimethylpentan-2-yl)amino)-1H-imidazo[1,2-b]pyrazole-7-carboxamide.

8. The bicyclic imidazo[1,2-b]pyrazole carboxamide and carbothioamide derivatives of general formula (V) and pharmaceutically acceptable salts thereof according to claim 1 as listed as follows:
Secondary carboxamide and carbothioamide derivatives and pharmaceutically salt thereof:
2-(Tert-butyl)-N-methyl-3-((2,4,4-trimethylpentan-2-yl)amino)-1H-imidazo[1,2-b]pyrazole-7-carbox-amide;
2-(Tert-butyl)-N-butyl-3-((2,4,4-trimethylpentan-2-yl)amino)-1H-imidazo[1,2-b]pyrazole-7-carbox-amide;
N,2-Di-tert-butyl-3-((2,4,4-trimethylpentan-2-yl)amino)-1H-imidazo[1,2-b]pyrazole-7-carboxamide;
2-(Tert-butyl)-N-cyclopropyl-3-((2,4,4-trimethylpentan-2-yl)amino)-1H-imidazo[1,2-b]pyrazole-7-carboxamide;
2-(Tert-butyl)-N-cyclopentyl-3-((2,4,4-trimethylpentan-2-yl)amino)-1H-imidazo[1,2-b]pyrazole-7-carboxamide;
(2-(Tert-butyl)-3-((2,4,4-trimethylpentan-2-yl)amino)-1H-imidazo[1,2-b]pyrazol-7-yl) (piperidin-1-yl)methanone;
(2-(Tert-butyl)-3-((2,4,4-trimethylpentan-2-yl)amino)-1H-imidazo[1,2-b]pyrazol-7-yl)(4-phenylpiperazin-1-yl)methanone;
N-Benzyl-2-(tert-butyl)-3-((2,4,4-trimethylpentan-2-yl)amino)-1H-imidazo[1,2-b]pyrazole-7-carboxamide;
2-(Tert-butyl)-N-phenyl-3-((2,4,4-trimethylpentan-2-yl)amino)-1H-imidazo[1,2-b]pyrazole-7-carboxamide;
2-(Tert-butyl)-N-(pyridin-2-yl)-3-((2,4,4-trimethylpentan-2-yl)amino)-1H-imidazo[1,2-b]pyrazole-7-carboxamide;
2-(Tert-butyl)-N-(pyridin-3-yl)-3-((2,4,4-trimethylpentan-2-yl)amino)-1H-imidazo[1,2-b]pyrazole-7-carboxamide;
2-(Tert-butyl)-N-(pyridin-4-yl)-3-((2,4,4-trimethylpentan-2-yl)amino)-1H-imidazo[1,2-b]pyrazole-7-carboxamide;
2-(Tert-butyl)-N-(thiazol-2-yl)-3-((2,4,4-trimethylpentan-2-yl)amino)-1H-imidazo[1,2-b]pyrazole-7-carboxamide;
2-(Tert-butyl)-N-(isoxazol-3-yl)-3-((2,4,4-trimethylpentan-2-yl)amino)-1H-imidazo[1,2-b]pyrazole-7-carboxamide;
2-(Tert-butyl)-N-(o-tolyl)-3-((2,4,4-trimethylpentan-2-yl)amino)-1H-imidazo[1,2-b]pyrazole-7-carbox-amide;
2-(Tert-butyl)-N-(3,5-dimethylphenyl)-3-((2,4,4-trimethylpentan-2-yl)amino)-1H-imidazo[1,2-b]pyrazole-7-carboxamide;
2-(Tert-butyl)-N-(4-isopropylphenyl)-3-((2,4,4-trimethylpentan-2-yl)amino)-1H-imidazo[1,2-b]pyrazole-7-carboxamide;
2-(Tert-butyl)-N-(4-methoxyphenyl)-3-((2,4,4-trimethylpentan-2-yl)amino)-1H-imidazo[1,2-b]pyrazole-7-carboxamide;
2-(Tert-butyl)-N-(2,4-dimethoxyphenyl)-3-((2,4,4-trimethylpentan-2-yl)amino)-1H-imidazo[1,2-b]pyrazole-7-carboxamide;
2-(Tert-butyl)-N-(2-(trifluoromethyl)phenyl)-3-((2,4,4-trimethylpentan-2-yl)amino)-1H-imidazo[1,2-b]pyrazole-7-carboxamide;
2-(Tert-butyl)-N-(3-(trifluoromethyl)phenyl)-3-((2,4,4-trimethylpentan-2-yl)amino)-1H-imidazo[1,2-b]pyrazole-7-carboxamide;
2-(Tert-butyl)-N-(4-(trifluoromethyl)phenyl)-3-((2,4,4-trimethylpentan-2-yl)amino)-1H-imidazo[1,2-b]pyrazole-7-carboxamide;
2-(Tert-butyl)-N-(2-fluorophenyl)-3-((2,4,4-trimethylpentan-2-yl)amino)-1H-imidazo[1,2-b]pyrazole-7-carboxamide;
2-(Tert-butyl)-N-(3-fluorophenyl)-3-((2,4,4-trimethylpentan-2-yl)amino)-1H-imidazo[1,2-b]pyrazole-7-carboxamide;
2-(Tert-butyl)-N-(4-fluorophenyl)-3-((2,4,4-trimethylpentan-2-yl)amino)-1H-imidazo[1,2-b]pyrazole-7-carboxamide 2-(Tert-butyl)-N-(4-chlorophenyl)-3-((2,4,4-trimethylpentan-2-yl)amino)-1H-imidazo[1,2-b]pyrazole-7-carboxamide;
N-(4-Bromophenyl)-2-(tert-butyl)-3-((2,4,4-trimethylpentan-2-yl)amino)-1H-imidazo[1,2-b]pyrazole-7-carboxamide 2-(Tert-butyl)-N-(4-nitrophenyl)-3-((2,4,4-trimethylpentan-2-yl)amino)-1H-imidazo[1,2-b]pyrazole-7-carboxamide;
2-(Tert-butyl)-N-(4-cyanophenyl)-3-((2,4,4-trimethylpentan-2-yl)amino)-1H-imidazo[1,2-b]pyrazole-7-carboxamide;
Ethyl 4-(2-(tert-butyl)-3-((2,4,4-trimethylpentan-2-yl)amino)-1H-imidazo[1,2-b]pyrazole-7-carbox-amido)benzoate;
2-(Tert-butyl)-N-(4-(methylthio)phenyl)-3-((2,4,4-trimethylpentan-2-yl)amino)-1H-imidazo[1,2-b]pyrazole-7-carboxamide;
2-(Tert-butyl)-N-(4-(dimethylamino)phenyl)-3-((2,4,4-trimethylpentan-2-yl)amino)-1H-imidazo[1,2-b]pyrazole-7-carboxamide;
2-(Tert-butyl)-N-(2,4-difluorophenyl)-3-((2,4,4-trimethylpentan-2-yl)amino)-1H-imidazo[1,2-b]pyrazole-7-carboxamide;
2-(Tert-butyl)-N-(3,4-difluorophenyl)-3-((2,4,4-trimethylpentan-2-yl)amino)-1H-imidazo[1,2-b]pyrazole-7-carboxamide;
2-(Tert-butyl)-N-(4-fluorophenyl)-3-((tert-butyl-2-yl)amino)-1H-imidazo[1,2-b]pyrazole-7-carboxamide;
2-(Tert-butyl)-N-(4-fluorophenyl)-3-((cyclohexyl-2-yl)amino)-1H-imidazo[1,2-b]pyrazole-7-carboxamide;
2-(Tert-butyl)-3-(tert-butylamino)-N-(4-fluorophenyl)-6-methyl-1H-imidazo[1,2-b]pyrazole-7-carboxamide;
2-(Tert-butyl)-3-(tert-butylamino)-N-(4-(trifluoromethyl)phenyl)-1H-imidazo[1,2-b]pyrazole-7-carboxamide;
2-(Tert-butyl)-3-(tert-butylamino)-N-(3-(trifluoromethyl)phenyl)-1H-imidazo[1,2-b]pyrazole-7-carboxamide;
2-(Tert-butyl)-3-(tert-butylamino)-N-(4-chloro-3-(trifluoromethyl)phenyl)-1H-imidazo[1,2-b]pyrazole-7-carboxamide;
2-(Tert-butyl)-3-(tert-butyl(methyl)amino)-N-(4-fluorophenyl)-1H-imidazo[1,2-b]pyrazole-7-carboxamide;
2-(Tert-butyl)-3-(tert-butylamino)-N-(5-fluoropyridin-2-yl)-1H-imidazo[1,2-b]pyrazole-7-carboxamide;
Methyl 2-((7-((4-fluorophenyl)carbamoyl)-2-(4-(trifluoromethyl)phenyl)-1H-imidazo[1,2-b]pyrazol-3-yl)amino)acetate;
Methyl 2-((2-(4-fluoro-3-(trifluoromethyl)phenyl)-7-((4-fluorophenyl)carbamoyl)-1H-imidazo[1,2-b]pyrazol-3-yl)amino)acetate;
Methyl 2-((2-(2,4-bis(trifluoromethyl)phenyl)-7-((4-fluorophenyl)carbamoyl)-1H-imidazo[1,2-b]pyrazol-3-yl)amino)acetate;
Methyl 2-((2-(3,5-bis(trifluoromethyl)phenyl)-7-((4-fluorophenyl)carbamoyl)-1H-imidazo[1,2-b]pyrazol-3-yl)amino)acetate;
2-(Tert-butyl)-N-(4-fluorobenzyl)-3-((2,4,4-trimethylpentan-2-yl)amino)-1H-imidazo[1,2-b]pyrazole-7-carboxamide;
2-(Tert-butyl)-N-(5-fluoropyridin-2-yl)-3-((2,4,4-trimethylpentan-2-yl)amino)-1H-imidazo[1,2-b]pyrazole-7-carboxamide;
2-(Tert-butyl)-N-(6-fluoropyridin-3-yl)-3-((2,4,4-trimethylpentan-2-yl)amino)-1H-imidazo[1,2-b]pyrazole-7-carboxamide;
2-(Tert-butyl)-N-(4-fluorophenyl)-N-methyl-3-((2,4,4-trimethylpentan-2-yl)amino)-1H-imidazo[1,2-b]pyrazole-7-carboxamide;
2-(Tert-butyl)-N-(4-fluorophenyl)-6-methyl-3-((2,4,4-trimethylpentan-2-yl)amino)-1H-imidazo[1,2-b]pyrazole-7-carboxamide;
2-(Tert-butyl)-N-(4-fluorophenyl)-3-(methyl(2,4,4-trimethylpentan-2-yl)amino)-1H-imidazo[1,2-b]pyrazole-7-carboxamide;
2-(Tert-butyl)-N-(6-fluoropyridin-3-yl)-3-(methyl(2,4,4-trimethylpentan-2-yl) amino)-1H-imidazo[1,2-b]pyrazole-7-carboxamide;
2-(Tert-butyl)-3-(methyl(2,4,4-trimethylpentan-2-yl)amino)-N-(thiazol-2-yl)-1H-imidazo[1,2-b]pyrazole-7-carboxamide;
Methyl 2-((7-((4-fluorophenyl)carbamoyl)-2-(4-(trifluoromethoxy)phenyl)-1H-imidazo[1,2-b]pyrazol-3-yl)amino)acetate;
N-(2-(Tert-butyl)-3-(tert-butylamino)-1H-imidazo[1,2-b]pyrazol-7-yl)-4-fluoro-benzamide;
3-(Tert-butylamino)-2-cyclopropyl-N-(4-fluorophenyl)-1H-imidazo[1,2-b]pyrazole-7-carbox-amide;
N-(4-bromophenyl)-2-(tert-butyl)-3-(tert-butylamino)-1H-imidazo[1,2-b]pyrazole-7-carbox-amide;
2-(Tert-butyl)-3-(tert-butylamino)-N-(4-nitrophenyl)-1H-imidazo[1,2-b]pyrazole-7-carboxamide;
3-(Tert-butylamino)-2-cyclopropyl-N-(4-nitrophenyl)-1H-imidazo[1,2-b]pyrazole-7-carbox-amide;
2-(Tert-butyl)-3-(tert-butylamino)-N-(2-methyl-4-nitrophenyl)-1H-imidazo[1,2-b]pyrazole-7-carboxamide;
2-(Tert-butyl)-3-(tert-butylamino)-N-(3-hydroxy-4-nitrophenyl)-1H-imidazo[1,2-b]pyrazole-7-carboxamide;
2-(Tert-butyl)-N-(3-hydroxy-4-nitrophenyl)-3-((2,4,4-trimethylpentan-2-yl)amino)-1H-imidazo[1,2-b]pyrazole-7-carboxamide;
N-(4-Aminophenyl)-2-(tert-butyl)-3-(tert-butylamino)-1H-imidazo[1,2-b]pyrazole-7-carboxamide;
2-(Tert-butyl)-3-(tert-butylamino)-N-(4-(dimethylamino)phenyl)-1H-imidazo[1,2-b]pyrazole-7-carboxamide; N-(4-aminophenyl)-3-(tert-butylamino)-2-cyclopropyl-1H-imidazo[1,2-b]pyrazole-7-carbox-amide;
N-(4-Amino-3-hydroxyphenyl)-2-(tert-butyl)-3-(tert-butylamino)-1H-imidazo[1,2-b]pyrazole-7-carboxamide;
N-(4-amino-2-methylphenyl)-2-(tert-butyl)-3-(tert-butylamino)-1H-imidazo[1,2-b]pyrazole-7-carboxamide;
2-(Tert-butyl)-3-(tert-butylamino)-N-(4-(methylthio)phenyl)-1H-imidazo[1,2-b]pyrazole-7-carboxamide;
N-(4-aminophenyl)-2-(tert-butyl)-3-((2,4,4-trimethylpentan-2-yl)amino)-1H-imidazo[1,2-b]pyrazole-7-carboxamide;
2-(Tert-butyl)-3-(tert-butylamino)-N-(4-hydroxyphenyl)-1H-imidazo[1,2-b]pyrazole-7-carboxamide;
2-(Tert-butyl)-N-(4-hydroxyphenyl)-3-((2,4,4-trimethylpentan-2-yl)amino)-1H-imidazo[1,2-b]pyrazole-7-carboxamide;
2-(Tert-butyl)-3-(cyclohexylamino)-N-(4-hydroxyphenyl)-1H-imidazo[1,2-b]pyrazole-7-carboxamide;
3-(cyclohexylamino)-N-(4-hydroxyphenyl)-2-(4-(trifluoromethyl)phenyl)-1H-imidazo[1,2-b]pyrazole-7-carboxamide;
2-(Tert-butyl)-N-(2-hydroxyphenyl)-3-((2,4,4-trimethylpentan-2-yl)amino)-1H-imidazo[1,2-b]pyrazole-7-carboxamide;
2-(tert-butyl)-N-(3-hydroxyphenyl)-3-((2,4,4-trimethylpentan-2-yl)amino)-1H-imidazo[1,2-b]pyrazole-7-carboxamide;
2-(Tert-butyl)-N-(4-hydroxy-2-methylphenyl)-3-((2,4,4-trimethylpentan-2-yl) amino)-1H-imidazo[1,2-b]pyrazole-7-carboxamide;
2-(Tert-butyl)-N-(4-fluorophenyl)-3-imino-2,3-dihydro-1H-imidazo[1,2-b]pyrazole-7-carboxamide;
2-(Tert-butyl)-N-(4-fluorobenzoyl)-3-imino-2,3-dihydro-1H-imidazo[1,2-b]pyrazole-7-carboxamide;
2-(Tert-butyl)-3-(tert-butylamino)-N-(4-fluorophenyl)-1H-imidazo[1,2-b]pyrazole-7-carbothioamide;
Ethyl 4-(2-(tert-butyl)-3-(tert-butylamino)-1H-imidazo[1,2-b]pyrazole-7-carbox-amido)benzoate;
2-(Tert-butyl)-3-(tert-butylamino)-N-(4-(5-((3aS,4S,6aR)-2-oxohexahydro-1H-thieno[3,4-d]imidazol-4-yl)pentanamido)phenyl)-1H-imidazo[1,2-b]pyrazole-7-carboxamide;
2-(tert-butyl)-3-(tert-butylamino)-N-(3-(trifluoromethyl)phenyl)-1H-imidazo[1,2-b]pyrazole-7-carboxamide;
2-(tert-butyl)-N-(4-hydroxyphenyl)-3-(pentylamino)-1H-imidazo[1,2-b]pyrazole-7-carboxamide;
2-(Tert-butyl)-3-(cyclohexylamino)-N-(4-hydroxyphenyl)-1H-imidazo[1,2-b]pyrazole-7-carbox-amide;
2-(Tert-butyl)-3-(tert-butylamino)-N-(4-hydroxyphenyl)-1H-imidazo[1,2-b]pyrazole-7-carboxamide;
N-(4-aminophenyl)-2-(tert-butyl)-3-((2,4,4-trimethylpentan-2-yl)amino)-1H-imidazo[1,2-b]pyrazole-7-carboxamide;
2-(Tert-butyl)-3-(tert-butylamino)-N-(4-(methylthio)phenyl)-1H-imidazo[1,2-b]pyrazole-7-carboxamide;
2-(Tert-butyl)-3-(tert-butylamino)-N-(4-(dimethylamino)phenyl)-1H-imidazo[1,2-b]pyrazole-7-carboxamide;
2-(Tert-butyl)-N-(4-fluorophenyl)-3-((cyclohexyl-2-yl)amino)-1H-imidazo[1,2-b]pyrazole-7-carboxamide;
2-(Tert-butyl)-N-(4-fluorophenyl)-3-((tert-butyl-2-yl)amino)-1H-imidazo[1,2-b]pyrazole-7-carboxamide;
2-(Tert-butyl)-N-(isoxazol-3-yl)-3-((2,4,4-trimethylpentan-2-yl)amino)-1H-imidazo[1,2-b]pyrazole-7-carboxamide;
2-(Tert-butyl)-N-(thiazol-2-yl)-3-((2,4,4-trimethylpentan-2-yl)amino)-1H-imidazo[1,2-b]pyrazole-7-carboxamide, and
2-(Tert-butyl)-N-(pyridin-3-yl)-3-((2,4,4-trimethylpentan-2-yl)amino)-1H-imidazo[1,2-b]pyrazole-7-carboxamide.

9. The bicyclic imidazo[1,2-b]pyrazole carboxamide derivatives according to claim 1 as listed detailed as follows:
2-(Tert-butyl)-3-(cyclohexylamino)-N-(4-hydroxyphenyl)-1H-imidazo[1,2-b]pyrazole-7-carbox-amide;
2-(Tert-butyl)-N-(4-hydroxyphenyl)-3-((2,4,4-trimethylpentan-2-yl)amino)-1H-imidazo[1,2-b]pyrazole-7-carboxamide;
2-(Tert-butyl)-3-(tert-butylamino)-N-(4-hydroxyphenyl)-1H-imidazo[1,2-b]pyrazole-7-carboxamide;
N-(4-aminophenyl)-2-(tert-butyl)-3-((2,4,4-trimethylpentan-2-yl)amino)-1H-imidazo[1,2-b]pyrazole-7-carboxamide;
2-(Tert-butyl)-3-(tert-butylamino)-N-(4-(methylthio)phenyl)-1H-imidazo[1,2-b]pyrazole-7-carboxamide;
2-(Tert-butyl)-3-(tert-butylamino)-N-(4-(dimethylamino)phenyl)-1H-imidazo[1,2-b]pyrazole-7-carboxamide;
N-(4-Aminophenyl)-2-(tert-butyl)-3-(tert-butylamino)-1H-imidazo[1,2-b]pyrazole-7-carboxamide;
2-(Tert-butyl)-N-(4-fluorophenyl)-3-((cyclohexyl-2-yl)amino)-1H-imidazo[1,2-b]pyrazole-7-carboxamide;
2-(Tert-butyl)-N-(4-fluorophenyl)-3-((tert-butyl-2-yl)amino)-1H-imidazo[1,2-b]pyrazole-7-carboxamide;
2-(Tert-butyl)-N-(4-fluorophenyl)-3-((2,4,4-trimethylpentan-2-yl)amino)-1H-imidazo[1,2-b]pyrazole-7-carboxamide;
2-(Tert-butyl)-N-(4-(dimethylamino)phenyl)-3-((2,4,4-trimethylpentan-2-yl)amino)-1H-imidazo[1,2-b]pyrazole-7-carboxamide;
2-(Tert-butyl)-N-(2,4-difluorophenyl)-3-((2,4,4-trimethylpentan-2-yl)amino)-1H-imidazo[1,2-b]pyrazole-7-carboxamide;
2-(Tert-butyl)-N-(isoxazol-3-yl)-3-((2,4,4-trimethylpentan-2-yl)amino)-1H-imidazo[1,2-b]pyrazole-7-carboxamide;
2-(Tert-butyl)-N-(thiazol-2-yl)-3-((2,4,4-trimethylpentan-2-yl)amino)-1H-imidazo[1,2-b]pyrazole-7-carboxamide;
2-(Tert-butyl)-N-(pyridin-3-yl)-3-((2,4,4-trimethylpentan-2-yl)amino)-1H-imidazo[1,2-b]pyrazole-7-carboxamide, and
2-(Tert-butyl)-N-phenyl-3-((2,4,4-trimethylpentan-2-yl)amino)-1H-imidazo[1,2-b]pyrazole-7-carboxamide.

10. The bicyclic imidazo[1,2-b]pyrazole carboxamide derivatives of general formula (IV) and pharmaceutically acceptable salts thereof according to claim 1 as listed as follows:
2-(Tert-butyl)-3-(cyclohexylamino)-N-(4-hydroxyphenyl)-1H-imidazo[1,2-b]pyrazole-7-carbox-amide;
2-(Tert-butyl)-N-(4-hydroxyphenyl)-3-((2,4,4-trimethylpentan-2-yl)amino)-1H-imidazo[1,2-b]pyrazole-7-carboxamide;
2-(Tert-butyl)-3-(tert-butylamino)-N-(4-hydroxyphenyl)-1H-imidazo[1,2-b]pyrazole-7-carboxamide;
N-(4-aminophenyl)-2-(tert-butyl)-3-((2,4,4-trimethylpentan-2-yl)amino)-1H-imidazo[1,2-b]pyrazole-7-carboxamide;
2-(Tert-butyl)-3-(tert-butylamino)-N-(4-(methylthio)phenyl)-1H-imidazo[1,2-b]pyrazole-7-carboxamide;
2-(Tert-butyl)-3-(tert-butylamino)-N-(4-(dimethylamino)phenyl)-1H-imidazo[1,2-b]pyrazole-7-carboxamide;
N-(4-Aminophenyl)-2-(tert-butyl)-3-(tert-butylamino)-1H-imidazo[1,2-b]pyrazole-7-carboxamide;
2-(Tert-butyl)-N-(4-fluorophenyl)-3-((cyclohexyl-2-yl)amino)-1H-imidazo[1,2-b]pyrazole-7-carboxamide;
2-(Tert-butyl)-N-(4-fluorophenyl)-3-((tert-butyl-2-yl)amino)-1H-imidazo[1,2-b]pyrazole-7-carboxamide;
2-(Tert-butyl)-N-(4-fluorophenyl)-3-((2,4,4-trimethylpentan-2-yl)amino)-1H-imidazo[1,2-b]pyrazole-7-carboxamide;
2-(Tert-butyl)-N-(4-(dimethylamino)phenyl)-3-((2,4,4-trimethylpentan-2-yl)amino)-1H-imidazo[1,2-b]pyrazole-7-carboxamide;
2-(Tert-butyl)-N-(2,4-difluorophenyl)-3-((2,4,4-trimethylpentan-2-yl)amino)-1H-imidazo[1,2-b]pyrazole-7-carboxamide;
2-(Tert-butyl)-N-(isoxazol-3-yl)-3-((2,4,4-trimethylpentan-2-yl)amino)-1H-imidazo[1,2-b]pyrazole-7-carboxamide;
2-(Tert-butyl)-N-(thiazol-2-yl)-3-((2,4,4-trimethylpentan-2-yl)amino)-1H-imidazo[1,2-b]pyrazole-7-carboxamide;
2-(Tert-butyl)-N-(pyridin-3-yl)-3-((2,4,4-trimethylpentan-2-yl)amino)-1H-imidazo[1,2-b]pyrazole-7-carboxamide; and
2-(Tert-butyl)-N-phenyl-3-((2,4,4-trimethylpentan-2-yl)amino)-1H-imidazo[1,2-b]pyrazole-7-carboxamide.

11. Medicinal and/or pharmaceutical compositions comprising the bicyclic imidazo[1,2-b]pyrazole carboxamide or carbothioamide derivatives of general formula (V) and pharmaceutically acceptable salts thereof according to claim 1.

12. The Medicinal and/or pharmaceutical compositions according to claim 10 further comprising inert, pharmaceutically acceptable, solid or liquid carriers and/or excipients.

13. The Medicinal and/or pharmaceutical compositions according to claim 11, wherein the composition is solid, semi-solid or liquid.

14. The Medicinal and/or pharmaceutical compositions according to claim 11, wherein the composition is tablet, inhalation powder, capsule, suppository or solution for injection.

15. A process for the preparation of medicinal and/or pharmaceutical compositions comprising mixing the bicyclic imidazo[1,2-b]pyrazole carboxamide or carbothioamide derivatives of general formula (V) and pharmaceutically acceptable salts thereof according to claim 1 with pharmaceutically applicable inert, solid or liquid carriers and/or excipients and formulating the resulting mixture into a medicinal and/or pharmaceutical composition by using standard formulation technics.

16. A novel process for the preparation of novel bicyclic imidazo[1,2-b]pyrazole carboxamide or carbothioamide derivatives of general formula (V) and pharmaceutically acceptable salts thereof according to claim 1, comprising:
reacting a precursor aminopyrazole of general formula (I), where X represents an O atom or of general formula (I′), and where X represents an S atom is synthesized from cyanoacetic acid derivative in a three steps manner, with the most diverse aldehydes (II) and isonitriles (III) in the presence of perchloric acid, called general “method A” or trifluoroacetic acid, called general “method B” to form compounds of the general formula (V) according to claim 1, 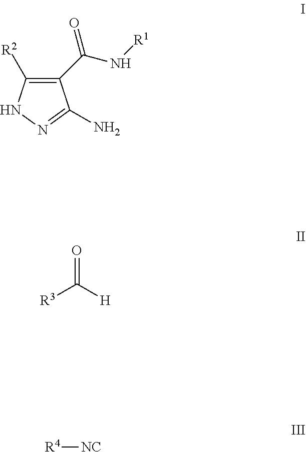
wherein in general formulas (I), (I′), (II) and (III) R₁ to R₄ and X represent the same groups of the general formula (V), and 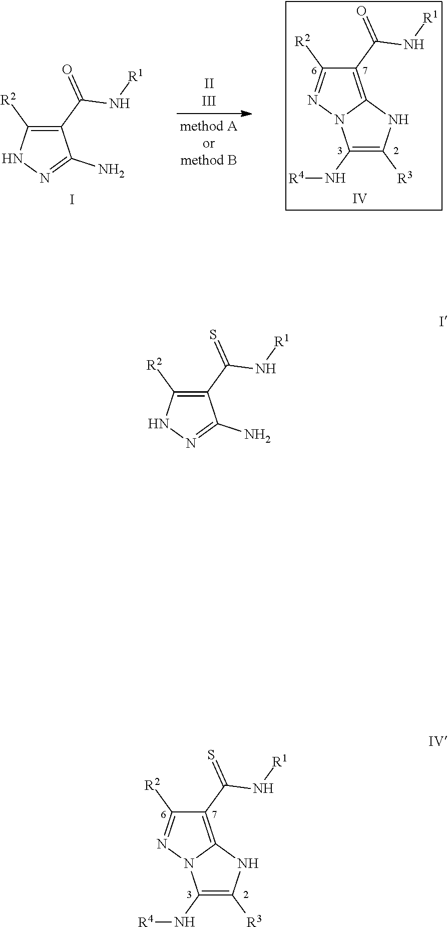
isolating due to the optimized conditions, most of the compounds of general formula (IV) and (IV′) according to claim 1 by simple filtration, and
converting the compounds of the general formula (V) into their pharmaceutically acceptable salts with physiologically tolerated acids.

17. The process according to the claim 16 further comprising using hydrochloric acid, acetic acid, oxalic acid, tartaric acid, mandelic acid, fumaric acid, lactic acid, citric acid for converting into the pharmaceutically accepted salts.

18. The process according to the claim 16, wherein the obtained compound is of general formula IV according to claim 1.

19. The process according to the claim 15, wherein the obtained compound is of general formula (IV′) according to claim 1.

20. The process according to claim 16 wherein, in the general process “method A”, a suspension of pyrazole of general formulas (I) or (I′) (0.50 mmol) in MeCN or THF (0.5 mL) aldehyde of general formula (II) (0.55 mmol), HClO4 (20 mol %), and isocyanide of general formula (III) (0.55 mmol) were added and stirred at room temperature for 6 h., then the crude mixture was purified by filtration followed by washing with cold MeCN or by column chromatography on silica gel (eluent: hexane/EtOAc or chloroform/methanol gradient) to afford pure products of general formulas (IV) or (IV′).

21. The process according to claim 16 wherein, in the general process “method B”, a suspension of pyrazole of general formulas (I) or (I′) (0.50 mmol) in EtOH/water mixture (1:1, 1 mL) aldehyde of general formula (II) (0.55 mmol), TFA (20 mol %), and isocyanide of general formula (III) (0.55 mmol) were added and stirred at room temperature for 15 minutes. Then the desired compound of general formulas (IV) or (IV′) was isolated by simple filtration followed by washing with water, then with EtOH.

22-35. (canceled)
